(19) United States
(12) Patent Application Publication
(10) Pub. No.: US 2025/0375380 A1
(43) Pub. Date: Dec. 11, 2025

(54) HIGH STRENGTH SINGLE UNIT DOSE FORMULATIONS AND METHODS OF USE THEREOF

(71) Applicant: Praxis Precision Medicines, Inc., Boston, MA (US)

(72) Inventor: Ahmad Hashash, Boston, MA (US)

(21) Appl. No.: 19/312,128

(22) Filed: Aug. 27, 2025

Related U.S. Application Data
(63) Continuation of application No. PCT/US2024/017961, filed on Mar. 1, 2024.
(60) Provisional application No. 63/449,529, filed on Mar. 2, 2023.

(51) Int. Cl. A61K 9/20; A61K 9/28; A61K 31/445

(52) U.S. Cl. CPC A61K 9/2054; A61K 9/2009; A61K 9/2018; A61K 9/2027; A61K 9/2813; A61K 9/284; A61K 9/2853; A61K 31/445

(57) ABSTRACT

Described herein, in part, are single-unit dosage forms, comprising the compound of Formula (I) or a pharmaceutically acceptable salt thereof (e.g., the compound of Formula (II), e.g., PRAX-944 HCl), wherein the composition is bioequivalent to a reference composition of the same dosage strength administered as multiple small, round tablets. The present invention further comprises methods useful for treating a disease or condition relating to aberrant function or activity of a T-type calcium channel, such as tremor (e.g., essential tremor).

### RELATED APPLICATIONS

This application is a continuation of International Patent Application No. PCT/US2024/017961, filed on Mar. 1, 2024; which claims priority to U.S. Provisional Application No. 63/449,529, filed on Mar. 2, 2023. The entire contents of each of the foregoing applications are hereby incorporated herein by reference.

### BACKGROUND

Essential tremor (ET) is the most common adult movement disorder, affecting up to 2% of the United States population (approximately 7 million Americans). ET is characterized by a 5 to 12 Hz postural and kinetic tremor (i.e., tremor during voluntary movement) in the upper limbs. The most characteristic clinical feature is kinetic tremor of the arms and hands, but tremor may also occur in the head and voice, and less commonly face, legs, and trunk. The diagnosis of ET is based on medical history and neurological exam as described in The International Parkinson and Movement Disorders Society Consensus Statement on the Classification of Tremors.

There is a range of severity of ET; some patients require no treatment, whereas others have severe disability with impairment in activities of daily living such as dressing and eating. ET, by definition, is generally not associated with other neurological signs, although there is increasing recognition that ET can also be associated with additional motor features, such as postural instability, dystonia, mild to moderate gait ataxia, and eye motion abnormalities. ET also is associated with an elevated prevalence of comorbid psychiatric disorders, including anxiety and depression. ET often worsens over time, with more severe tremor over the course of years to decades and corresponding worsening disability.

ET can be sporadic, but a family history of an autosomal-dominant pattern of inheritance is commonly encountered and importantly, variants in the calcium voltage-gated channel subunit alpha1 G (CACNAIG) gene, which encodes the T-type Ca²⁺ channel isoform Cav3.1 have been identified as the cause of ET in at least three families. The importance of the CACNA1G gene to cerebellar development and function is further highlighted by the observation that variants in this gene can also cause childhood cerebellar atrophy and spinocerebellar ataxia type 42. The functional consequences of these genetic variants are consistent with the expression of T-type Ca²⁺ channels in the cerebellum and the distal cerebello-thalamo-cortical (CTC) circuit and their physiological contribution to the oscillatory burst firing in the thalamus that is synchronized with and likely drives the clinically observable tremor.

Propranolol is the only Food and Drug Administration (FDA)-approved orally administered treatment indicated for the treatment of ET. Propranolol was originally developed for hypertension, and in the treatment of ET exhibits limited efficacy with side effects (e.g., bradycardia) that often lead to discontinuation. The unmet medical need in ET has resulted in the off-label use of medications in multiple drug classes, including anticonvulsants, barbiturates, benzodiazepines, antipsychotics, and others, with limited appreciable therapeutic benefit for patients with ET. A recent evidenced based review concluded that only propranolol, primidone, and topiramate had sufficient evidence to support efficacy amongst 28 drugs studied for ET. Surgical interventions that interrupt cerebello-thalamo-cortical (CTC) burst activity, such as deep brain stimulation or focused ultrasound, are effective treatments for ET but carry risks, such as sensory disturbances, hemiparesis, dysarthria, ataxia, gait disturbances, delirium, cognitive decline, tissue damage, venous thromboembolic events, and intracerebral hemorrhage.

Voltage-gated ion channels play a critical role in maintaining the delicate balance between neuronal excitation and inhibition within neuronal networks. Alterations of voltage-gated ion channels, particularly T-Type Ca²⁺ channels have been implicated in ET. Comprising three isoforms (CaV3.1, CaV3.2, and CaV3.3), T-type Ca²⁺ channels are expressed widely throughout the brain, especially in the CTC circuit. This class of calcium channels has been shown to play a critical role in modulating neuronal firing patterns by controlling the switch between a tonic, steady firing pattern to one consisting of brief high-frequency bursts of activity in wild-type rodents, and in rodent models of tremor. Importantly, aberrant bursts in the CTC circuit occur at the same frequency as upper limb tremor in ET, suggesting that reduction of T-type-calcium channel-mediated burst firing may have therapeutic benefit in reducing tremor-related burst firing in the CTC circuit.

PRAX-944 (N-((1-(2-(tert-butylamino)-2-oxoethyl) piperidin-4-yl)methyl)-3-chloro-5-fluorobenzamide hydrochloride) is a high-affinity, state-dependent, selective inhibitor of T-type Ca²⁺ channels being developed for the treatment of ET. In vitro electrophysiology studies have demonstrated that PRAX-944 has specificity and nanomolar potency for T-type Ca²⁺ channels (CaV3.1 and CaV3.3).

By blocking T-type Ca²⁺ channels, PRAX-944 may reduce burst activity in the CTC circuit and in turn reduce tremor and associated disability. PRAX-944 has shown robust activity in an animal model of ET. Thus far, the PRAX-944 program has completed 5 Phase 1 studies in healthy participants using dose levels from 2.5 mg to 120 mg. Recent clinical trials with PRAX-944 have used the MR7 formulation and introduced a titration regimen for achieving higher dose levels. Together, these developments have contributed to improved safety and tolerability, as well as a wider therapeutic window compared with the immediate release (IR) formulation. The MR7 formulation, which was designed to delay tmax and reduce Cmax and have minimal effects on AUC, was associated with a reduction in the frequency of CNS, psychiatric, and overall AEs compared to the IR formulation in 2 trials, Z944-103 and Z944-104. Furthermore, the PRAX-944-105 trial established that doses above 40 mg are well tolerated when administered in a 28-day titration regimen. A maximum tolerated dose has not been observed for the MR7 formulation administered in this fashion and dosing up to 120 mg is well-tolerated. The program has also collected preliminary efficacy data in an early Phase 2 trial in participants with ET.

With a modified release (MR) formulation that supports once daily (QD) dosing over a 24-fold well-tolerated dose range that exhibits robust pharmacodynamic effects, in combination with preliminary efficacy data, there is a clear clinical rationale to support further study of the safety and efficacy of PRAX-944 for addressing the unmet need in ET.

However, certain challenges associated with the use of the currently available PRAX-944 MR formulations (i.e., placebo, 5 mg, and 20 mg tablets) have been identified during clinical trials. For example, for clinical blinding purposes, different dose strengths of PRAX-944 are provided in multi-tablet combinations (e.g., including one or more of placebo, 5 mg, and 20 mg tablets) packaged in blisters. It has been found that opening blister packages, handling small, round tablets, and taking many pills is especially challenging for patients struggling with movement disorders, such as essential tremor (ET). Accordingly, there is a need to develop new single unit dose tablets of, suitable strengths to improve usability for patients, reduce the total number of dosage units (e.g., tablets) needed to deliver a specific dosage strength to a patient, and reduce or eliminate the risk of dosing errors.

### SUMMARY

This disclosure provides compositions, including single-unit dosage forms and pharmaceutical compositions, comprising the compound of Formula (I) or a pharmaceutically acceptable salt thereof (e.g., the compound of Formula (II), e.g., PRAX-944 HCl). In certain embodiments, the compositions described herein are bioequivalent to currently available oral dosage forms, such as the current PRAX-944 dosage form which is a modified release (MR) formulation available as 5-mg and 20-mg tablets that are round and small in size (e.g., about 6 mm in diameter). The present invention further comprises methods useful for treating a disease or condition relating to aberrant function or activity of a T-type calcium channel, such as tremor (e.g., essential tremor). The present disclosure further comprises methods useful for preventing and/or treating a disease or condition relating to aberrant function or activity of a T-type calcium channel, such as tremor, such as essential tremor.

In one aspect, the present disclosure provides a single-unit dosage form, comprising: the compound of Formula (I) or a pharmaceutically acceptable salt thereof (e.g., the compound of Formula (II), e.g., PRAX-944 HCl), wherein the single-unit dosage form is bioequivalent to a reference composition of the same dosage strength administered as one or more dosage forms, such as multiple small, round 20-mg PRAX-944 tablets.

In some embodiments, the single-unit dosage form and the reference composition have: (i) a different size; (ii) a different shape; (iii) both a different size and a different shape; (iv) the same shape but different sizes; or (v) the same size but different shapes. In certain embodiments, the single-unit dosage form is larger than the reference composition. In some embodiments, the reference composition comprises smaller, round 20-mg PRAX-944 tablets.

In some embodiments, the single-unit dosage form is a larger tablet that exhibits bioequivalence upon administration to a subject in a fed state and/or fasted state as compared to administration of a reference composition of the same dosage strength administered as one or more smaller dosage forms, optionally one or more small, round 20-mg PRAX-944 tablets, to a subject in a fed and/or fasted state; wherein bioequivalency is established by: (a) a 90% Confidence Interval for AUC which is between about 80% and about 125% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, or about 125%), and (b) a 90% Confidence Interval for Cmax, which is between about 80% and about 125% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, or about 125%). In some embodiments, the 90% confidence interval for the AUC and/or Cmax may be contained within tighter confidence limits, e.g., about 90% to about 120%, about 90% to about 115%, about 90% to about 110%, or about 90% to about 100%.

In some embodiments, the single-unit dosage form (i) reduces the total number of dosage units (e.g., tablets) needed to deliver a specific dosage strength to a subject; (ii) is characterized by a physical size and shape, (e.g., tablet size and shape) which makes it easier and more convenient for subjects with a movement disorder, such as essential tremor (ET), to grasp; (iii) can be formulated as single-unit dose strengths for 5 mg, 10 mg, 20 mg, 40 mg, 60 mg, 80 mg, 100 mg, and 120 mg; and/or (iv) does not require titration to reach doses >40 mg. In certain embodiments, titration may reduce and/or eliminate adverse events. In some embodiments, titration may comprise the administration of one or more single-unit dosages forms described herein. In some embodiments, at least about 50% (e.g., at least about 50%, at least about 55%, at least about 60%, at least about 65%, at least about 70%, at least about 75%, at least about 80%, at least about 85%, at least about 90%, at least about 95%, or about 100%) of the compound of Formula (I) or a pharmaceutically acceptable salt thereof (e.g., the compound of Formula (II), e.g., PRAX-944 HCl) is released within a predetermined time period upon administration to a subject. In certain embodiments, the predetermined time period may range from about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours).

In some embodiments, at least about 50% (e.g., at least about 50%, at least about 55%, at least about 60%, at least about 65%, at least about 70%, at least about 75%, at least about 80%, at least about 85%, at least about 90%, at least about 95%, or about 100%) of the compound of Formula (I) or a pharmaceutically acceptable salt thereof (e.g., the compound of Formula (II), e.g., PRAX-944 HCl) is released within about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours) upon administration to a subject.

In some embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the compound of Formula (I) or a pharmaceutically acceptable salt thereof (e.g., the compound of Formula (II), e.g., PRAX-944 HCl) is released within about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours) upon administration to a subject.

In some embodiments, about 5% to about 25% of the compound of Formula (I) or a pharmaceutically acceptable salt thereof (e.g., the compound of Formula (II), e.g., PRAX-944 HCl) is released within about 1 to about 2 hours upon administration to a subject.

In some embodiments, about 25% to about 50% of the compound of Formula (I) or a pharmaceutically acceptable salt thereof (e.g., the compound of Formula (II), e.g., PRAX-944 HCl) is released within about 2 hours to about 4 hours upon administration to a subject.

In some embodiments, about 50% to about 75% of the compound of Formula (I) or a pharmaceutically acceptable salt thereof (e.g., the compound of Formula (II), e.g., PRAX-944 HCl) is released within about 3 hours to about 7 hours upon administration to a subject.

In some embodiments, about 75% to about 100% of the compound of Formula (I) or a pharmaceutically acceptable salt thereof (e.g., the compound of Formula (II), e.g., PRAX-944 HCl) is released within about 6 hours to about 10 hours upon administration to a subject.

In some embodiments, about 80% of the compound of Formula (I) or a pharmaceutically acceptable salt thereof (e.g., the compound of Formula (II), e.g., PRAX-944 HCl) is released within about 7 hours upon administration to a subject.

In some embodiments, about 90% of the compound of Formula (I) or a pharmaceutically acceptable salt thereof (e.g., the compound of Formula (II), e.g., PRAX-944 HCl) is released within about 9 hours to about 12 hours upon administration to a subject.

In some embodiments, at least about 50% (e.g., at least about 50%, at least about 55%, at least about 60%, at least about 65%, at least about 70%, at least about 75%, at least about 80%, at least about 85%, at least about 90%, at least about 95%, or about 100%) of the compound of Formula (I) or a pharmaceutically acceptable salt thereof (e.g., the compound of Formula (II), e.g., PRAX-944 HCl) is released within a predetermined time period using USP apparatus type-I, media containing 900 mL 0.1 M HCl, and a paddle speed of 100 rpm. In certain embodiments, the predetermined time period may range from about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours).

In some embodiments, at least about 50% (e.g., at least about 50%, at least about 55%, at least about 60%, at least about 65%, at least about 70%, at least about 75%, at least about 80%, at least about 85%, at least about 90%, at least about 95%, or about 100%) of the compound of Formula (I) or a pharmaceutically acceptable salt thereof (e.g., the compound of Formula (II), e.g., PRAX-944 HCl) is released in about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours) using USP apparatus type-I, media containing 900 mL 0.1 M HCl, and a paddle speed of 100 rpm. In some embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the compound of Formula (I) or a pharmaceutically acceptable salt thereof (e.g., the compound of Formula (II), e.g., PRAX-944 HCl) is released in about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours) using USP apparatus type-I, media containing 900 mL 0.1 M HCl, and a paddle speed of 100 rpm.

In some embodiments, the single-unit dosage form comprises from about 1 mg to about 200 mg of the compound of Formula (I) or a pharmaceutically acceptable salt thereof (e.g., the compound of Formula (II), e.g., PRAX-944 HCl). In some embodiments, the single-unit dosage form comprises from about 1% by weight to about 70% by weight of the compound of Formula (I) or a pharmaceutically acceptable salt thereof (e.g., the compound of Formula (II), e.g., PRAX-944 HCl).

In some embodiments, the single-unit dosage form further comprises a modified-release polymer. In some embodiments, the modified-release polymer comprises a matrix polymer, optionally selected from the group consisting of a hydrophilic matrix polymer, a hydrophobic matrix polymer, a polyacrylate polymer, and combinations thereof. In some embodiments, the modified-release polymer comprises (i) a hydrophilic matrix polymer, optionally selected from the group consisting of hypromellose, HPMC (hydroxyl-propyl methylcellulose), and combinations thereof, optionally wherein the HPMC (hydroxyl-propyl methylcellulose) is selected from the group consisting of Methocel K4M, Methocel K100LV, Methocel E50LV, and combinations thereof; (ii) a hydrophobic matrix polymer, optionally selected from the group consisting of ethyl cellulose, ethocel, and combinations thereof; and/or (iii) a polyacrylate polymer, optionally selected from the group consisting of Eudragit RL100, Eudragit RS100, and combinations thereof. In some embodiments, the single-unit dosage form comprises from about 5 mg to 300 mg of a modified-release polymer. In some embodiments, the single-unit dosage form comprises from about 10% by weight to about 70% by weight of the modified-release polymer. In some embodiments, the modified-release polymer is hypromellose.

In some embodiments, the single-unit dosage form further comprises a diluent, optionally a soluble diluent. In some embodiments, the diluent comprises: (i) a cellulose derivative, optionally a microcrystalline cellulose, optionally a silicified microcrystalline cellulose; (ii) a starch, optionally selected from the group consisting of a hydrolyzed starch, a pregelatinized starch, and combinations thereof); (iii) an anhydrous lactose; (iv) a lactose monohydrate; (v) a di-calcium phosphate (DCP); and/or (vi) a sugar alcohol, optionally selected from the group consisting of sorbitol, xylitol, mannitol, and combinations thereof. In some embodiments, the single-unit dosage form comprises from about 5 mg to about 300 mg of diluent. In some embodiments, the single-unit dosage form comprises from about 5% by weight to about 50% by weight of diluent. In some embodiments, the diluent is microcrystalline cellulose, optionally silicified microcrystalline cellulose. In some embodiments, the diluent is a sugar alcohol, optionally mannitol.

In some embodiments, the single-unit dosage form further comprises a glidant. In some embodiments, the glidant is selected from the group consisting of fumed silica, optionally colloidal silicon dioxide, talc, magnesium carbonate, and combinations thereof. In some embodiments, the single-unit dosage form comprises about 1 mg to about 10 mg of glidant. In some embodiments, the single-unit dosage form comprises from about 1% by weight to about 10% by weight of glidant. In some embodiments, the glidant is fumed silica, optionally colloidal silicon dioxide.

In some embodiments, the single-unit dosage form further comprises a lubricant. In some embodiments, lubricant is selected from the group consisting of magnesium stearate, calcium stearate, stearic acid, talc, silica, a fat, optionally vegetable stearin, and combinations thereof. In some embodiments, the single-unit dosage form comprises from about 1 mg to about 10 mg of lubricant. In some embodiments, the single-unit dosage form comprises from about 1% by weight to about 10% by weight of lubricant. In some embodiments, the lubricant comprises magnesium stearate.

In some embodiments, the single-unit dosage form further comprises a coating. In some embodiments, the coating comprises a film coating agent. In some embodiments, the coating comprises compendial grade polyvinyl alcohol, titanium dioxide, polyethylene glycol 3350, and/or talc. In some embodiments, the single-unit dosage form comprises from about 1 mg to about 20 mg of coating. In some embodiments, the single-unit dosage form comprises from about 1% by weight to about 10% by weight of coating. In some embodiments, the coating comprises Opadry® II white 85F18422.

In some embodiments, the single-unit dosage form comprises a tablet, optionally formulated for oral administration. In some embodiments, the single-unit dosage form comprises an oblong tablet, an oval tablet, or a capsule-shaped tablet, optionally wherein the tablet is not a round tablet. In some embodiments, opening blister packages of oblong, oval, or capsule-shaped tablets, handling oblong, oval, or capsule-shaped tablets, and taking one or more oblong, oval, or capsule-shaped tablets is less challenging and move convenient for patients struggling with movement disorders, such as essential tremor (ET), e.g., as compared to a small, round tablet (e.g., about 6 mm in diameter).

In some embodiments, the single-unit dosage form comprises a total weight from about 200 mg to about 600 mg per dosage unit (e.g., per tablet). In some embodiments, the single-unit dosage form comprises about 1% to about 100% by weight of PRAX-944 HCl, optionally wherein the total weight of the dosage unit (e.g., per tablet) is from about 200 mg to about 600 mg (e.g., about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, or about 600 mg).

In some embodiments, the single-unit dosage form comprises a length of about 1 mm to about 30 mm (e.g., a length of about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, about 20.5 mm, about 21 mm, about 21.5 mm, about 22 mm, about 22.5 mm, about 23 mm, about 23.5 mm, about 24 mm, about 24.5 mm, about 25 mm, about 25.5 mm, about 26 mm, about 26.5 mm, about 27 mm, about 27.5 mm, about 28 mm, about 28.5 mm, about 29 mm, about 29.5 mm, or about 30 mm).

In some embodiments, the single-unit dosage form comprises a width of about 1 mm to about 30 mm (e.g., a width of about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, about 20.5 mm, about 21 mm, about 21.5 mm, about 22 mm, about 22.5 mm, about 23 mm, about 23.5 mm, about 24 mm, about 24.5 mm, about 25 mm, about 25.5 mm, about 26 mm, about 26.5 mm, about 27 mm, about 27.5 mm, about 28 mm, about 28.5 mm, about 29 mm, about 29.5 mm, or about 30 mm).

In some embodiments, the single-unit dosage form comprises a width of about 1 mm to about 10 mm (e.g., a width of about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, or about 10 mm).

In some embodiments, the single-unit dosage form comprises a length of about 14 to about 16 mm (e.g., a length of about 14 mm, about 14.1 mm, about 14.2 mm, about 14.3 mm, about 14.4 mm, about 14.5 mm, about 14.6 mm, about 14.7 mm, about 14.8 mm, about 14.9 mm, about 15 mm, about 15.1 mm, about 15.2 mm, about 15.3 mm, about 15.4 mm, about 15.5 mm, about 15.6 mm, about 15.7 mm, about 15.8 mm, about 15.9 mm, or about 16 mm), and a width of about 5 mm to about 7 mm (e.g., a width of about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, or about 7 mm).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 5 mg of PRAX-944 free base per dosage unit (e.g., per tablet), optionally from about 1 mg to about 10 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 10 mg of PRAX-944 free base per dosage unit (e.g., per tablet), optionally from about 5 mg to about 15 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 15 mg of PRAX-944 free base per dosage unit (e.g., per tablet), optionally from about 10 mg to about 20 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 20 mg of PRAX-944 free base per dosage unit (e.g., per tablet), optionally from about 15 mg to about 25 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 25 mg of PRAX-944 free base per dosage unit (e.g., per tablet), optionally from about 20 mg to about 30 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 30 mg of PRAX-944 free base per dosage unit (e.g., per tablet), optionally from about 25 mg to about 35 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 35 mg of PRAX-944 free base per dosage unit (e.g., per tablet), optionally from about 30 mg to about 40 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 40 mg of PRAX-944 free base per dosage unit (e.g., per tablet), optionally from about 35 mg to about 45 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 45 mg of PRAX-944 free base per dosage unit (e.g., per tablet), optionally from about 40 mg to about 50 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 50 mg of PRAX-944 free base per dosage unit (e.g., per tablet), optionally from about 45 mg to about 55 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 55 mg of PRAX-944 free base per dosage unit (e.g., per tablet), optionally from about 50 mg to about 60 mg of PRAX-944 HCl per dosage unit (e.g., per tablet). In some embodiments, the single-unit dosage form comprises the compound of Formula (I) or a pharmaceutically acceptable salt thereof (e.g., the compound of Formula (II), e.g., PRAX-944 HCl) in an amount equivalent to about 60 mg of PRAX-944 free base per dosage unit (e.g., per tablet), optionally from about 55 mg to about 65 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 65 mg of PRAX-944 free base per dosage unit (e.g., per tablet), optionally from about 60 mg to about 70 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 70 mg of PRAX-944 free base per dosage unit (e.g., per tablet), optionally from about 65 mg to about 75 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 75 mg of PRAX-944 free base per dosage unit (e.g., per tablet), optionally from about 70 mg to about 80 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 80 mg of PRAX-944 free base per dosage unit (e.g., per tablet), optionally from about 75 mg to about 85 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 85 mg of PRAX-944 free base per dosage unit (e.g., per tablet), optionally from about 80 mg to about 90 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 90 mg of PRAX-944 free base per dosage unit (e.g., per tablet), optionally from about 85 mg to about 95 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 95 mg of PRAX-944 free base per dosage unit (e.g., per tablet), optionally from about 90 mg to about 100 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 100 mg of PRAX-944 free base per dosage unit (e.g., per tablet), optionally from about 95 mg to about 105 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 105 mg of PRAX-944 free base per dosage unit (e.g., per tablet), optionally from about 100 mg to about 110 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 110 mg of PRAX-944 free base per dosage unit (e.g., per tablet), optionally from about 105 mg to about 115 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 115 mg of PRAX-944 free base per dosage unit (e.g., per tablet), optionally from about 110 mg to about 120 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 120 mg of PRAX-944 free base per dosage unit (e.g., per tablet), optionally from about 115 mg to about 125 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 125 mg of PRAX-944 free base per dosage unit (e.g., per tablet), optionally from about 120 mg to about 130 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 130 mg of PRAX-944 free base per dosage unit (e.g., per tablet), optionally from about 125 mg to about 135 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 135 mg of PRAX-944 free base per dosage unit (e.g., per tablet), optionally from about 130 mg to about 140 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 140 mg of PRAX-944 free base per dosage unit (e.g., per tablet), optionally from about 135 mg to about 145 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 145 mg of PRAX-944 free base per dosage unit (e.g., per tablet), optionally from about 140 mg to about 150 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 150 mg of PRAX-944 free base per dosage unit (e.g., per tablet), optionally from about 145 mg to about 155 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 155 mg of PRAX-944 free base per dosage unit (e.g., per tablet), optionally from about 150 mg to about 160 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 160 mg of PRAX-944 free base per dosage unit (e.g., per tablet), optionally from about 155 mg to about 165 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 1.0 mg to about 200 mg (e.g., about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of PRAX-944 free base per dosage unit (e.g., per tablet). In some embodiments, the single-unit dosage form comprises from about 1% by weight to about 50% (e.g., about 5%, about 10%, about 15%, about 20%, about 25%, about 30%, about 35%, about 40%, about 45%, or about 50%) by weight of PRAX-944 HCl, optionally wherein the total dosage unit (e.g., tablet) weight is from about 50 mg to about 500 mg (e.g., about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 5.0 mg of PRAX-944 free base per dosage unit (e.g., per tablet). In some embodiments, the single-unit dosage form comprises from about 1% by weight to about 5% by weight of PRAX-944 HCl, optionally from about 1 mg to about 10 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 10.0 mg of PRAX-944 free base per dosage unit (e.g., per tablet). In some embodiments, the single-unit dosage form comprises from about 1% by weight to about 10% by weight of PRAX-944 HCl, optionally from about 5 mg to about 15 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 20.0 mg of PRAX-944 free base per dosage unit (e.g., per tablet). In some embodiments, the single-unit dosage form comprises from about 1% by weight to about 10% by weight of PRAX-944 HCl, optionally from about 15 mg to about 25 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 40.0 mg of PRAX-944 free base per dosage unit (e.g., per tablet). In some embodiments, the single-unit dosage form comprises from about 5% by weight to about 15% by weight of PRAX-944 HCl, optionally from about 25 mg to about 50 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 60.0 mg of PRAX-944 free base per dosage unit (e.g., per tablet). In some embodiments, the single-unit dosage form comprises from about 5% by weight to about 20% by weight of PRAX-944 HCl, optionally from about 50 mg to about 75 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 80.0 mg of PRAX-944 free base per dosage unit (e.g., per tablet). In some embodiments, the single-unit dosage form comprises from about 15% by weight to about 25% by weight of PRAX-944 HCl, optionally from about 75 mg to about 100 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 100.0 mg of PRAX-944 free base per dosage unit (e.g., per tablet). In some embodiments, the single-unit dosage form comprises from about 20% by weight to about 25% by weight of PRAX-944 HCl, optionally from about 75 mg to about 125 mg of PRAX-944 HCl per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 120.0 mg of PRAX-944 free base per dosage unit (e.g., per tablet). In some embodiments, the single-unit dosage form comprises from about 25% by weight to about 35% by weight of PRAX-944 HCl, optionally from about 110 mg to about 135 mg of PRAX-944 HCl per dosage unit (e.g., per tablet).

In some embodiments, the single-unit dosage form further comprises one or more of (i) a modified-release polymer, optionally from about 35% by weight to about 45% by weight of modified-release polymer, optionally from about 175 mg to about 185 mg of modified-release polymer per dosage unit (e.g., per tablet); (i) a soluble diluent, optionally from about 6% by weight to about 10% by weight of soluble diluent, optionally from about 34 mg to about 38 mg of soluble diluent per dosage unit (e.g., per tablet); (iii) a glidant, optionally from about 1% by weight to about 3% by weight of glidant, optionally from about 6 mg to about 8 mg of glidant per dosage unit (e.g., per tablet); (iv) a lubricant, optionally from about 0.5% by weight to about 2% by weight of lubricant, optionally from about 3 mg to about 6 mg of lubricant per dosage unit (e.g., per tablet); and (v) a coating, optionally from about 12% by weight to about 15% by weight of coating, optionally from about 1 mg to about 5 mg of coating per dosage unit (e.g., per tablet).

In some embodiments, the single-unit dosage form comprises: (i) large tablet prototype 01 (58% K4M Round) (DC); (ii) large tablet prototype 01 (58% K4M Caplet) (DC); (iii) large tablet prototype 04 (58% K4M) (DG); (iv) large tablet prototype 23 (50% K4M); (v) large tablet prototype 28 (58% K100LV); (vi) large tablet prototype 34 (50% K100LV, 8% Mannitol EG); (vii) large tablet prototype 36 (50% K100LV, 8% Mannitol IG); (viii) large tablet prototype 52 (28% K100LV, 30% E50LV); (ix) large tablet prototype 53 (40% K100LV, 8% Mannitol IG); or (x) large tablet prototype 54 (46% K100LV, 12% Mannitol IG). In some embodiments, the single-unit dosage form comprises: (i) large tablet prototype 04 (58% K4M) (DG); (ii) large tablet prototype 28 (58% K100LV); or (iii) large tablet prototype 53 (40% K100LV, 8% Mannitol IG).

In one aspect, the present disclosure provides a pharmaceutical composition comprising the single-unit dosage form of any one of the preceding claims, and at least one pharmaceutically acceptable carrier or excipient.

In one aspect, the present disclosure provides a method of treating a disease or condition relating to aberrant function or activity of a T-type calcium channel in a subject in need thereof, comprising administering to the subject a therapeutically effective amount of the single-unit dosage or the pharmaceutical composition described herein.

In one aspect, the present disclosure provides a method of treating a tremor in a subject in need thereof, comprising administering to the subject a therapeutically effective amount of the single-unit dosage or the pharmaceutical composition described herein.

In one aspect, the present disclosure provides a method of treating an essential tremor in a subject in need thereof, comprising administering to the subject a therapeutically effective amount of the single-unit dosage or the pharmaceutical composition described herein.

In some embodiments, the method results in reduction of the essential tremor as assessed by The Essential Tremor Rating Assessment Scale (TETRAS) score. In some embodiments, the reduction of the essential tremor is assessed by The Essential Tremor Rating Assessment Scale (TETRAS) upper limb score. In some embodiments, the reduction of the essential tremor is assessed by TETRAS-ADL (activities of daily living). In some embodiments, the reduction of the essential tremor is assessed by TETRAS performance subscale score or TETRAS performance individual items. In some embodiments, the method results in reduction of the essential tremor as assessed by accelerometer-based upper limb score. In some embodiments, the method results in reduction of sigma frequency band. In some embodiments, the essential tremor is upper limb tremor.

Other objects and advantages will be apparent to those skilled in the art based upon consideration of the ensuing Brief Description of the Drawings, Detailed Description, Examples, and Claims.

### BRIEF DESCRIPTION OF THE DRAWINGS

FIG. 1 depicts composition and characteristics of active pharmaceutical ingredient, PRAX-944 (also referred to herein as PRAX-944 HCl).

FIG. 2A depicts tablets of various shapes and sizes.

FIG. 2B depicts a comparison between the currently available PRAX-944 dosage form, which is a modified release (MR) formulation available as 5-mg and 20-mg round tablets that are small in size (6 mm in diameter), and an exemplary bioequivalent single-unit dosage form described herein.

FIG. 3A depicts the PRAX-944-107 study design. N-number of participants planned for the total population; n=number of participants planned per arm. Fasted: Each dose of study drug will be taken with 240 mL of water after an overnight fast. No food or drink, except water, will be allowed for at least 10 hours prior to dosing. Participants are to remain fasted for 4 hours post dose. Participants are not allowed to drink water for 2 hours before and for 1 hour after each dose. Fed: Study drug co-administered with high-fat, high-calorie meal.

FIG. 3B depicts study drugs administered in PRAX-944-107.

FIG. 3C depicts exemplary high dose strength PRAX-944 tablet formulations tested in 944-107 study.

FIG. 3D depicts a schematic of an exemplary Phase 3 clinical trial study design to evaluate the efficacy of the large, single-unit dosage PRAX-944 tablets (20 mg, 40 mg, 80 mg, and 120 mg) compared to the same dose levels delivered with the small, round 20-mg PRAX-944 tablets (1, 2, 4, and 6 tablets, respectively).

FIGS. 4A-4B depict in vitro (FIG. 4A) and in vivo (FIG. 4B) release profiles for 20 mg strength PRAX-944 modified release (MR) tablet and immediate release (IR) capsule. For improved tolerability, PRAX-944 is formulated as a modified release (MR) tablet to extend drug release and absorption, and thus, blunt Cₘₐₓ and prolong plasma concentration-time profile compared to an immediate release (IR) dosage form. As shown in FIG. 4A, about 100% of the PRAX-944 is released within about 1 hour for the immediate release (IR) dosage form; and about 80% of the PRAX-944 is released within about 7 hours for the modified release (MR) dosage form.

As shown in FIG. 4B, the IR formulation achieved a Cₘₐₓ (ng/mL) (CV %) of 131 (25.6) and an AUC_{∞} (ng·hr/mL) (CV %) of 1090 (24.4); and the MR formulation achieved a Cₘₐₓ (ng/mL) (CV %) of 47.1 (34.3) and an AUC_{∞} (ng·hr/mL) (CV %) of 1010 (34.2).

FIG. 5A depicts in vitro release profiles for PRAX-944 large tablet prototypes (i.e., large tablet prototype 01 (58% K4M Round) (DC), large tablet prototype 01 (58% K4M Caplet) (DC), large tablet prototype 04 (58% K4M) (DG), large tablet prototype 23 (50% K4M), large tablet prototype 28 (58% K100LV), large tablet prototype 34 (50% K100LV, 8% Mannitol EG), large tablet prototype 36 (50% K100LV, 8% Mannitol IG), large tablet prototype 52 (28% K100LV, 30% E50LV), large tablet prototype 53 (40% K100LV, 8% Mannitol IG), and large tablet prototype 54 (46% K100LV, 12% Mannitol IG)) compared to reference tablet (i.e., 20 mg small, round clinical tablet). DC and DG designate direct compression and dry granulation, respectively.

FIG. 5B depicts in vitro release profiles for PRAX-944 large tablet prototypes (i.e., large tablet prototype 04, large tablet prototype 28, and large tablet prototype 53) compared to reference tablet (i.e., 20 mg small, round clinical tablet).

FIG. 5C depicts in vitro release profiles for PRAX-944 MR tablets (i.e., 20 mg large tablet, 40 mg large tablet, 80 mg large tablet, and 120 mg large tablet) compared to reference tablet (i.e., 20 mg small, round clinical tablet).

FIGS. 6A-6B depict relative bioavailability of 120 mg large tablets (test) compared to small, round tablets (reference) by cross-over analysis. By crossover analysis, the 120 mg large and small, round tablets meet bioequivalence criteria (90% CI within 80-125%) for AUCₗₐₛₜ and Cₘₐₓ (FIG. 6B). Outlier subjects excluded from analysis.

FIG. 7 depicts dose proportionality with small, round tablets, or large tablets. Outlier subjects excluded from analysis.

FIGS. 8A-8B depict lack of food effect with 120 mg PRAX-944. Coadministration of the 120 mg large tablet with a high-fat, high-calorie meal results in a delay in tₘₐₓ of 3 hours and a slight increase in AUC and Cₘₐₓ but within the no effect boundaries (90% CI within 80-125%). Outlier subjects excluded from analysis.

FIGS. 9A-9C depict relative bioavailability by parallel analysis with small, round tablets, or large tablets. While the large tablets appear to have increased exposure relative to the small, round tablets this is likely an artifact of the parallel study design. When the relative bioavailability of 120 mg tablets is assessed by crossover analysis, bioequivalence (BE) criteria is met, while when assessed by parallel analysis, the large tablet has higher exposure than the small, round tablets, by a similar magnitude as the tablets at lower strengths. Outlier subjects excluded from analysis.

FIG. 10A depicts an exemplary schematic of a crossover bioequivalence study.

### DETAILED DESCRIPTION

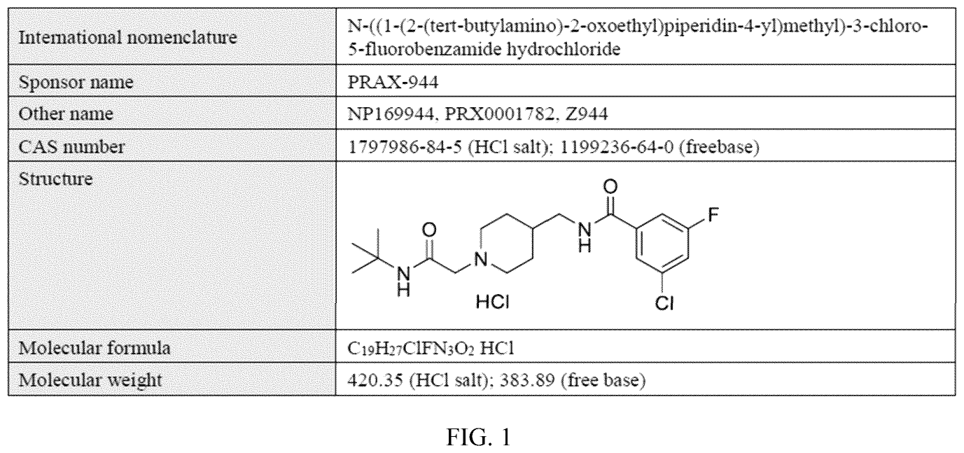

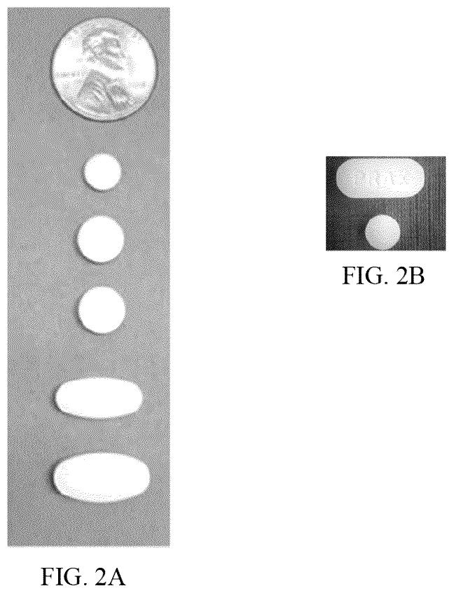

This disclosure provides compositions designed to provide a larger, more convenient PRAX-944 dosage form as compared to the currently available PRAX-944 dosage form, which is a modified release (MR) formulation available as 5-mg and 20-mg tablets that are round and small in size (6 mm in diameter). For example, the pharmaceutical compositions of the present disclosure are designed to minimize the number of tablets needed to deliver specific dose levels of PRAX-944 (FIG. 1) and to increase the physical size of the tablet and make it into a capsule shape to make it easier to grasp and more convenient, including more usable, for patients, especially those with essential tremor (ET). FIG. 2A shows tablets of various shapes and sizes, and FIG. 2B shows a comparison between the currently available PRAX-944 dosage form, which is a modified release (MR) formulation available as 5-mg and 20-mg tablets that are round and small in size (6 mm in diameter), and an exemplary bioequivalent single-unit dosage form described herein. Further, the pharmaceutical compositions of the present disclosure comprising larger capsule-shaped tablets have substantially the same bioavailability, e.g., with respect to the rate (maximum plasma drug concentration; Cmax) and extent (area under the plasma concentration-time curve; AUC) of absorption of PRAX-944 as compared to multiple small, round 20-mg tablets.

### Definitions

In general, the “effective amount” of a compound refers to an amount sufficient to elicit the desired biological response. As will be appreciated by those of ordinary skill in this art, the effective amount of a compound may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or reduce, or eliminate one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances therapeutic efficacy of another therapeutic agent.

As used herein, the term “refractory” refers to a disease, disorder, or condition that does not readily yield or respond to therapy or treatment, or is not controlled by a therapy or treatment. In some embodiments, a disease, disorder, or condition described herein is refractory (e.g., refractory epilepsy or refractory absence seizures or essential tremor that is refractory) and does not respond to standard therapy or treatment.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. In certain embodiments, the terms “patient” and “subject” are used interchangeably herein. In some embodiments, the subjects are selected for treatment with the compound of formula (I) due to a clinical diagnosis of essential tremor. In some embodiments, the subjects selected for treatment with the compound of formula (I) have essential tremor, but do not have intention tremor.

The terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art; for example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include acetate, adipate, alginate, ascorbate, aspartate, benzenesulfonate, benzoate, besylate, bisulfate, borate, butyrate, camphorate, camphorsulfonate, citrate, cyclopentanepropionate, cyclamate, digluconate, dodecylsulfate, edisylate, ethanesulfonate, esylate, formate, fumarate, gentisate, glucoheptonate, glycerophosphate, gluconate, glucoronate, glutamate, glutarate, glycolate, hemisulfate, heptanoate, hexanoate, hippurate, hydroiodide, 2-hydroxy-ethanesulfonate, isethionate, ketoglutarate, lactobionate, lactate, laurate, lauryl sulfate, malate, maleate, malonate, mesylate, methanesulfonate, napadisylate, napsylate, 2-naphthalenesulfonate, nicotinate, nitrate, oleate, oxalate, oroate, palmitate, pamoate, pectinate, persulfate, 3-phenylpropionate, phosphate, picrate, pivalate, propionate, sebacate, stearate, succinate, sulfate, tartrate, thiocyanate, p-toluenesulfonate, undecanoate, valerate salts, stereoisomers thereof (e.g., enantiomers, diastereomers) and the like.

Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. In certain embodiments, a compound of Formula (I) is a hydrochloric acid salt.

The term “modified-release polymer” or “matrix polymer” refers to a polymer that is used in a formulation (e.g., tablets and capsules) to modify the release rate of the drug upon the administration to a subject. For example, a modified-release polymer is used to dissolve a drug over time in order to be released slower and steadier into the bloodstream. For example, a modified-release polymer is a controlled-release polymer. For example, a modified-release polymer or a controlled-release polymer is an HPMC polymer. In some embodiments, a modified-release polymer may include hydrophilic matrix polymers (e.g., hypromellose, HPMC (hydroxyl-propyl methylcellulose)), hydrophobic matrix polymers (e.g., ethyl cellulose, ethocel), or polyacrylate polymers (e.g., Eudragit RL100, Eudragit RS100).

The term “diluent” or “filler” as used herein refers to an excipient used to increase weight and improve content uniformity. For example, diluents include cellulose derivatives (e.g., microcrystalline cellulose), starches (e.g., hydrolyzed starches, and partially pregelatinized starches), anhydrous lactose, lactose monohydrate, di-calcium phosphate (DCP), sugar alcohols (e.g., sorbitol, xylitol and mannitol)).

The term “glidant” as used herein refers to an excipient used to promote powder flow by reducing interparticle friction and cohesion. For example, glidants include fumed silica (e.g., colloidal silicon dioxide), talc, and magnesium carbonate.

The term “lubricant” as used herein refers to an excipient used to prevent ingredients from clumping together and from sticking to the tablet punches or capsule filling machine. Lubricants are also used to ensure that tablet formation and ejection can occur with low friction between the solid and die wall. For example, lubricants include magnesium stearate, calcium stearate, stearic acid, talc, silica, and fats (e.g., vegetable stearin).

The term “coating” as used herein refers to an excipient to protect tablet ingredients from deterioration by moisture in the air and make large or unpleasant-tasting tablets easier to swallow. For example, coatings may include Opadry® II white 85F18422, which is comprised of compendial grade polyvinyl alcohol, titanium dioxide, polyethylene glycol 3350, and talc.

The term “bioavailability” refers to the extent to which an active moiety (e.g., a drug and/or a metabolite) is absorbed into the general circulation and becomes available at the site of drug action in the body.

The term “bioequivalence” or “bioequivalent” means that two drug products do not differ significantly when the two products are administered at the same dose under similar conditions. A product (e.g., a single-unit dosage form or a pharmaceutical composition as described herein) can be considered bioequivalent to a second product (e.g., a reference composition) if there is no significant difference in the rate and extent to which the active ingredient or active moiety becomes available at the site of drug action when the product is administered at the same molar dose as the second product under similar conditions in an appropriately designed study. Two products with different rates of absorption can be considered equivalent if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In some embodiments, bioequivalence may be based on no more than an about 20% difference between the AUC and Cmax of a product versus a second product. In some embodiments, bioequivalence may be based on no more than an about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, or about 20% difference between the AUC and Cmax of a product versus a second product.

In some embodiments, bioequivalence can be assumed when, for example, the 90% confidence interval ranges between about 80% and about 125% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, or about 125%) for the target parameters (e.g., Cmax and AUC). In some embodiments, the 90% confidence interval for the AUC and/or Cmax may be contained within tighter confidence limits, e.g., about 90% to about 120%, about 90% to about 115%, about 90% to about 110%, or about 90% to about 100%. In some embodiments, bioequivalence may be determined based on steady-state studies, and/or on single-dose studies. In some embodiments, bioequivalence studies may be conducted in fasted and/or fed states.

In certain embodiments, the compositions described herein are bioequivalent to currently available oral dosage forms, such as the current PRAX-944 dosage form which is a modified release (MR) formulation available as 5-mg and 20-mg tablets that are round and small in size (e.g., about 6 mm in diameter).

Accordingly, a test composition, such as a single-unit dosage form or a pharmaceutical composition as described herein, may be said to be bioequivalent to a reference composition if the 90% confidence interval of the mean value for the area under the curve (AUC) (AUCt or AUCinf) of the blood plasma levels of the test composition is within about 80% to about 125% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, or about 125%) of the corresponding mean value of the reference composition, and if the 90% confidence interval of the mean value for the maximum concentration (Cmax) of the blood plasma levels of the test composition is within about 80% to about 125% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, or about 125%) of the corresponding mean value of the reference composition. In some embodiments, the reference composition comprises PRAX-944 drug product supplied as 5 mg and 20 mg MR, white, round, film coated tablets. Each reference tablet may contain PRAX-944 HCl DS equivalent to either 5 mg or 20 mg of PRAX-944 free base and, optionally, the following inactive ingredients: microcrystalline cellulose, hypromellose, colloidal silicon dioxide, magnesium stearate, triacetin, titanium dioxide, and talc.

The terms “dose strength” and “dose level” are used interchangeably herein and may refer to the amount of an active agent, e.g., the compound of Formula (I) or a pharmaceutically acceptable salt thereof (e.g., the compound of Formula (II), e.g., PRAX-944 HCl), measured in units of concentration. In certain embodiments, the terms “dose strength” and “dose level” may refer to the amount of the active agent, e.g., the compound of Formula (I) or a pharmaceutically acceptable salt thereof (e.g., the compound of Formula (II), e.g., PRAX-944 HCl), as free base and not to the amount of salts or derivatives of the active agent which is included in the compositions, formulations, and dosage forms described herein.

The terms “dosage form” and “dosage unit” are used interchangeably herein and may refer to the physical form of a dose of an active agent, e.g., the compound of Formula (I) or a pharmaceutically acceptable salt thereof (e.g., the compound of Formula (II), e.g., PRAX-944 HCl), intended for administration or consumption. In some embodiments, the dosage form comprises a physically discrete unit suitable for dosing a subject, e.g., each unit containing a predetermined quantity and/or dose strength of an active agent, e.g., the compound of Formula (I) or a pharmaceutically acceptable salt thereof (e.g., the compound of Formula (II), e.g., PRAX-944 HCl), calculated to produce a desired therapeutic effect either alone or in combination with one or more additional dosage units. The route of administration (ROA) for delivery of an active agent may depend, e.g., on the dosage form of the active agent. In certain embodiments, the compositions, formulations, and dosage forms described herein may be formulated and intended for oral administration to a subject. In some embodiments, the compositions, formulations, and dosage forms described herein, which when formulated and intended for oral administration to a subject, may include capsules, tablets, pills, powders, lozenges, aqueous or oily suspensions, granules, emulsions, syrups, elixirs, and the like. In such dosage forms, the active agent, e.g., the compound of Formula (I) or a pharmaceutically acceptable salt thereof (e.g., the compound of Formula (II), e.g., PRAX-944 HCl), may be mixed with a pharmaceutically acceptable excipient or carrier.

The term “single unit dosage” and “single unit dosage form” are used interchangeably herein and may refer to compositions and formulations comprising an active agent, e.g., the compound of Formula (I) or a pharmaceutically acceptable salt thereof (e.g., the compound of Formula (II), e.g., PRAX-944 HCl), comprising one discrete dosage unit, e.g., one tablet. In some embodiments, the dosage forms, e.g., single unit dosage forms, described herein are bioequivalent to currently available PRAX-944 dosage forms, e.g., comprising a modified release (MR) formulation available as 5-mg and 20-mg round tablets having a small size (about 6 mm in diameter).

The term “multiple unit dosage” and “multiple unit dosage form” are used interchangeably herein and may refer to compositions and formulations comprising an active agent, e.g., the compound of Formula (I) or a pharmaceutically acceptable salt thereof (e.g., the compound of Formula (II), e.g., PRAX-944 HCl), comprising a plurality of dosage units, e.g., a plurality of tablets. In some embodiments, the currently available PRAX-944 dosage forms comprise multiple unit dosage forms, e.g., comprising a modified release (MR) formulation available as 5-mg and 20-mg round tablets having a small size (about 6 mm in diameter).

TABLE 1
Abbreviations

| Term or | |
|---|---|
| Abbreviation | Definition |
| ABC | adenosine triphosphate-binding cassette |
| ADLs | activities of daily living |
| AE | adverse event |
| AESI | adverse event of special interest |
| ALT | alanine aminotransferase |
| AST | aspartate aminotransferase |
| AIVR | accelerated idioventricular rhythm |
| APD | action potential duration |
| AUC | area under the concentration-time curve |
| AUC∞ | area under the plasma concentration-time |
| | curve from time zero to infinity |
| AUCₗₐₛₜ | area under the curve to last measurable concentration |
| AUCτ | area under the plasma concentration-time |
| | curve during a dosage interval |
| BCRP | breast cancer resistance protein |
| BDC | bile-duct-cannulated |
| BID | twice daily |
| BLQ | below the lower limit of quantification |
| BMI | body mass index |
| bpm | beats per minute |
| BUN | blood urea nitrogen |
| Cₘₐₓ | maximum plasma drug concentration |
| CRF | case report form |
| C-SSRS | Columbia-Suicide Severity Rating Scale |
| Ca²⁺ | calcium ion |
| Ca_{V} | voltage-gated calcium channels |
| Ca_{V}1.2 | L-type voltage-dependent calcium channel, |
| | alpha 1C subunit |
| Ca_{V}2.2 | N-type voltage-dependent calcium channel, |
| | alpha 1B subunit |
| Ca_{V}3.1 | L-type voltage-dependent calcium channel, |
| | alpha 1G subunit |
| Ca_{V}3.2 | L-type voltage-dependent calcium channel, |
| | alpha 1H subunit |
| Ca_{V}3.3 | L-type voltage-dependent calcium channel, |
| | alpha 1I subunit |
| CD-1 | cluster of differentiation 1 |
| Cₘₐₓ | maximum (peak) plasma drug concentration |
| CNS | central nervous system |
| CPN | chronic progressive nephropathy |
| CTC | cerebello-thalamo-cortical |
| CV % | coefficient of variation |
| CYP | cytochrome P450 |
| CYP1A2 | cytochrome P450 1A2 |
| CYP2B6 | cytochrome P450 2B6 |
| CYP2C8 | cytochrome P450 2C8 |
| CYP2C9 | cytochrome P450 2C9 |
| CYP2C19 | cytochrome P450 2C19 |
| CYP2D6 | cytochrome P450 2D6 |
| CYP3A4 | cytochrome P450 3A4 |
| DBS | deep brain stimulation |
| DMSO | dimethylsulfoxide |
| DRG | dorsal root ganglion |
| DS | drug substance |
| ECG | electrocardiogram |
| EOS | End of Study |
| EOT | End of Treatment |
| EEG | electroencephalography |
| ET | essential tremor |
| FDA | Food and Drug Administration |
| FSH | follicle-stimulating hormone |
| FOB | functional observation battery |
| GCP | Good Clinical Practice |
| GI | gastrointestinal |
| GLP | Good Laboratory Practice |
| HBsAg | hepatitis B surface antigen |
| hCa_{V} | human L-type voltage-dependent calcium channel |
| hCa_{V}3.1 | human L-type voltage-dependent calcium channel, |
| | alpha 1G subunit |
| hCa_{V}3.2 | human L-type voltage-dependent calcium channel, |
| | alpha 1H subunit |
| hCa_{V}3.3 | human L-type voltage-dependent calcium channel, |
| | alpha 1I subunit |
| HCl | hydrochloride |
| hERG | human ether-a-go-go |
| IB | Investigator's Brochure |
| IC₅₀ | half-maximal inhibitor concentration |
| I_{gut} | intestinal luminal concentration |
| IP | intraperitoneal |
| IR | immediate release |
| IV | intravenous |
| KIM-1 | kidney injury molecule-1 |
| MATE | multidrug and toxin extrusion protein |
| MDR1 | multidrug resistance 1 protein |
| MDS | International Parkinson and Movement Disorders Society |
| MR | modified release |
| mRNA | messenger ribonucleic acid |
| MTD | maximum tolerated dose |
| Na_{V} | voltage-gated sodium channel |
| Na_{V}1.5 | sodium channel protein type 5 subunit alpha |
| NOAEL | no-observed-adverse-effect-level |
| NREM | non-rapid eye movement |
| NSVT | non-sustained ventricular tachycardia |
| OAT | organic anion transporter |
| OATP | organic anion-transporting polypeptides |
| OCT | organic cation transporter |
| PBPK | physiologically based pharmacokinetic |
| P-gp | P-glycoprotein |
| PD | Parkinson's Disease |
| PK | pharmacokinetic(s) |
| PO | oral administration |
| PS | performance subscale |
| PSG | polysomnography |
| QD | once daily |
| QID | four times daily |
| RBD | REM sleep behavior disorder |
| rCa_{V} | rat N-type voltage-dependent calcium channel |
| rCa_{V}1.2 | rat N-type voltage-dependent calcium channel, |
| | alpha 1C subunit |
| rCa_{V}2.2 | rat N-type voltage-dependent calcium channel, |
| | alpha 1B subunit |
| rCYP | recombinant CYP |
| REM | rapid eye movement |
| SAE | serious adverse event |
| SLC | solute carrier |
| sLMA | spontaneous locomotor activity |
| SNc | substantia nigra pars compacta |
| STN | subthalamic nucleus |
| t1/2 | elimination half-life |
| TEAE | treatment-emergent adverse event |
| TETRAS | The Essential Tremor Rating Assessment Scale |
| TETRAS-PS | TETRAS performance subscale |
| TETRAS-UL | TETRAS upper limb |
| TK | toxicokinetic |
| tₘₐₓ | time to reach maximum (peak) plasma concentration |
| | following drug administration |
| US | United States |

### Modified-Release PRAX-944 Dosage Forms and Compositions

PRAX-944 (FIG. 1) is an orally active, high-affinity, state-dependent, selective inhibitor of T-type calcium (Ca2+) channels. The T-type Ca2+-channel family is comprised of three different isoforms CaV3.1, CaV3.2, and CaV3.3, which are widely but differentially expressed in the thalamocortical and cerebellar circuitry, critical pathways implicated in the development of, e.g., essential tremor (ET). PRAX-944 is being developed for the treatment of various diseases or conditions relating to aberrant function or activity of a T-type calcium channel, including, for example, essential tremor (ET). For evaluation in clinical trials, PRAX-944 is currently produced as 5 mg and 20 mg modified release (MR), film coated tablets and matching placebo for oral (PO), once daily (QD) administration. The MR mechanism is achieved through inclusion of a hydrophilic polymer that hydrates and gels in an aqueous medium to form a matrix that controls the release of the drug substance (DS) from the tablet. Each active tablet contains, e.g., PRAX-944 hydrochloride (HCl) DS equivalent to either 5 mg or 20 mg of PRAX-944 free base and the following inactive ingredients: microcrystalline cellulose, hypromellose, colloidal silicon dioxide, magnesium stearate, triacetin, titanium dioxide, and talc. PRAX-944 was previously produced as immediate release (IR) tablets, as described, e.g., in the first clinical trial, Z944-B01. The current 5 mg and 20 mg MR formulations were developed following the observation in the first clinical trial, Z944-B01, that central nervous system (CNS) and psychiatric adverse events (AEs) observed with a single daily dose of the IR formulation could be reduced by splitting it into multiple administrations over the course of several hours, as described, e.g., in the clinical trial, Z944-104, evaluating the safety and tolerability of the PRAX-944 MR7 formulation.

The PRAX-944 MR7 formulation currently being used in clinical trials was designed to release 80% of the active drug into solution in vitro after 7 hours. This formulation was selected for further development after comparing the formulation to other MR formulations with faster dissolution. In general, the previously studied MR formulations, produced as 5 mg and 20 mg MR, film-coated tablets, blunt Cₘₐₓ and and delay tₘₐₓ compared to the IR formulation but have minimal effects on the AUC. Additionally, the frequency of CNS, psychiatric, and overall AEs is reduced with the MR formulations, with larger reductions in AE frequency corresponding to the extent to which Cₘₐₓ is reduced, resulting in the selection of the MR7 formulation for ongoing clinical trials. Additionally, titration has been shown to improve the tolerability profile of PRAX-944 MR7. However, during clinical trials, certain challenges associated with the use of the currently available PRAX-944 MR formulations (i.e., placebo, 5 mg, and 20 mg MR tablets) have been identified. For example, for clinical blinding purposes, different dose strengths of PRAX-944 are provided in multi-tablet combinations (e.g., including one or more of placebo, 5 mg, and 20 mg tablets) packaged in blisters. It has been found that having to handle small round tablets, blister packages, and multiple pills is particularly challenging for patients struggling with movement disorders, such as essential tremor (ET). As such, there is a need to develop new single dosage unit, larger sized, high dose strength tablets with suitable shape to improve patients' comfort and convenience and reduce or eliminate chances for dosing errors.

Accordingly, in one aspect, the present disclosure provides compositions, including single-unit dosage forms, comprising a compound of Formula (I):

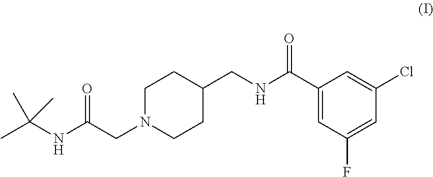 [SMILES: CC(C)(C)NC(=O)CN1CCC(CNC(=O)c2cc(F)cc(Cl)c2)CC1]

or a pharmaceutically acceptable salt thereof.

In some embodiments, the compound is the HCl salt of the compound of Formula (I), depicted below as Formula (II):

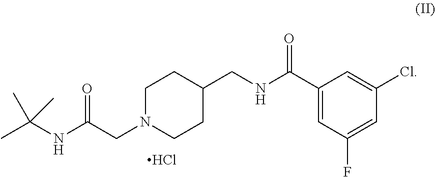 [SMILES: CC(C)(C)NC(=O)CN1CCC(CNC(=O)c2cc(F)cc(Cl)c2)CC1 ; Cl]

In some embodiments, the compound is the acetate salt, adipate salt, alginate salt, ascorbate salt, a sparatate salt, besylate salt, benzoate salt, citrate salt, cyclamate salt, edisylate salt, esylate salt, isethionate salt, fumarate salt, gentisate salt, gluconate salt, glucuronate salt, glutamate salt, glutarate salt, ketoglutarate salt, glycolate salt, hippurate salt, lactobionate salt, maleate salt, malate salt, malonate salt, mesylate salt, napadisylate salt, napsylate salt, oleate salt, oroate salt, oxalate salt, pamoate salt, phosphate salt, sebacate salt, succinate salt, or tartrate salt of the compound of Formula (I).

A compound of Formula (I) is described, for example, in PCT Publication WO2009/146540, incorporated by reference herein. Crystalline salts of a compound of Formula (I) are described, for example, in PCT Publication WO2021/007487, incorporated by reference herein. Deuterated analogs of a compound of Formula (I) are described, for example, in PCT Publication WO2022/099207, incorporated by reference herein. The safety profile, efficacy, tolerability, and pharmacokinetics of a compound of Formula (I) in modified release formulations with and without titration has been assessed, as described in PCT Publication WO2021/222342, incorporated by reference herein.

In some embodiments, the compound of formula (I) is in a crystalline form. In certain embodiments, the crystalline form is a crystalline form previously or contemporaneously described, such as crystalline Pattern B, crystalline Pattern C or crystalline Pattern D. Crystalline Pattern B and crystalline Pattern C are described, e.g., in WO 2021/007487, the entire contents of which are hereby incorporated herein by reference. Crystalline Pattern D is characterized by an x-ray powder diffraction (XRPD) pattern comprising at least one peak at the diffraction angle (° 2θ) selected from the group consisting of:
a peak at approximately 12.0°;
a peak at approximately 15.6°;
a peak at approximately 16.7°;
a peak at approximately 19.8°;
a peak at approximately 21.2°;
a peak at approximately 24.1°;
a peak at approximately 25.2°;
a peak at approximately 27.3°; and
a peak at approximately 30.2°.

In some embodiments, the compound of Formula (I) or a pharmaceutically acceptable salt thereof (e.g., the compound of Formula (II)) is PRAX-944 HCl (N-((1-(2-(tert-butylamino)-2-oxoethyl) piperidin-4-yl)methyl)-3-chloro-5-fluorobenzamide hydrochloride).

In some embodiments, the compound of Formula (I) or a pharmaceutically acceptable salt thereof (e.g., the compound of Formula (II), e.g., PRAX-944 HCl), may be in a dosage form, such as a single-unit dosage form, or in a pharmaceutical composition.

In one aspect, the present invention provides compositions, including single-unit dosage forms, comprising the compound of Formula (I) or a pharmaceutically acceptable salt thereof (e.g., the compound of Formula (II), e.g., PRAX-944 HCl).

In one aspect, the present invention provides modified-release dosage forms, including single-unit dosage forms, comprising the compound of Formula (I) or a pharmaceutically acceptable salt thereof (e.g., the compound of Formula (II), e.g., PRAX-944 HCl).

In one aspect, the present invention provides compositions, including single-unit dosage forms, comprising: the compound of Formula (I) or a pharmaceutically acceptable salt thereof (e.g., the compound of Formula (II), e.g., PRAX-944 HCl), wherein the composition is bioequivalent to a reference composition of the same dosage strength administered as multiple small, round 20-mg PRAX-944 tablets. In some embodiments, the compositions exhibit bioequivalence upon administration to a human subject, e.g., in a fed state and/or fasted state, as compared to administration of a reference composition of the same dosage strength administered as multiple small, round 20-mg PRAX-944 tablets to a human subject, e.g., in a fed and/or fasted state. In some embodiments, bioequivalency is established by: (i) a 90% Confidence Interval for AUC which is between about 80% and about 125% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, or about 125%), and (ii) a 90% Confidence Interval for Cmax, which is between about 80% and about 125% (e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, about 101%, about 102%, about 103%, about 104%, about 105%, about 106%, about 107%, about 108%, about 109%, about 110%, about 111%, about 112%, about 113%, about 114%, about 115%, about 116%, about 117%, about 118%, about 119%, about 120%, about 121%, about 122%, about 123%, about 124%, or about 125%). In some embodiments, the 90% confidence interval for the AUC and/or Cmax may be contained within tighter confidence limits, e.g., about 90% to about 120%, about 90% to about 115%, about 90% to about 110%, or about 90% to about 100%.

Certain embodiments of the present disclosure provide larger single-unit dose strength MR tablets (e.g., about 15 mm in length and about 6.5 mm in width) that are bioequivalent to smaller, round 20-mg tablets, e.g., at each of the dose levels used in the current titration regimen: 20 mg, 40 mg, 60 mg, 80 mg, 100 mg, and 120 mg.

In some embodiments, at least about 50% (e.g., at least about 50%, at least about 55%, at least about 60%, at least about 65%, at least about 70%, at least about 75%, at least about 80%, at least about 85%, at least about 90%, at least about 95%, or about 100%) of the compound of Formula (I) or a pharmaceutically acceptable salt thereof (e.g., the compound of Formula (II), e.g., PRAX-944 HCl) is released within a predetermined time period upon administration to a subject. In certain embodiments, the predetermined time period may range from about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours).

In some embodiments, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the compound of Formula (I) or a pharmaceutically acceptable salt thereof (e.g., the compound of Formula (II), e.g., PRAX-944 HCl) is released within about 1 hour to about 12 hours (e.g., about 1 hour, about 2 hours, about 3 hours, about 4 hours, about 5 hours, about 6 hours, about 7 hours, about 8 hours, about 9 hours, about 10 hours, about 11 hours, or about 12 hours) upon administration to a subject. In some embodiments, about 5% to about 25% of the compound of Formula (I) or a pharmaceutically acceptable salt thereof (e.g., the compound of Formula (II), e.g., PRAX-944 HCl) is released within about 1 to about 2 hours upon administration to a subject. In some embodiments, about 25% to about 50% of the compound of Formula (I) or a pharmaceutically acceptable salt thereof (e.g., the compound of Formula (II), e.g., PRAX-944 HCl) is released within about 2 hours to about 4 hours upon administration to a subject. In some embodiments, about 50% to about 75% of the compound of Formula (I) or a pharmaceutically acceptable salt thereof (e.g., the compound of Formula (II), e.g., PRAX-944 HCl) is released within about 3 hours to about 7 hours upon administration to a subject. In some embodiments, about 75% to about 100% of the compound of Formula (I) or a pharmaceutically acceptable salt thereof (e.g., the compound of Formula (II), e.g., PRAX-944 HCl) is released within about 6 hours to about 10 hours upon administration to a subject. In some embodiments, about 80% of the compound of Formula (I) or a pharmaceutically acceptable salt thereof (e.g., the compound of Formula (II), e.g., PRAX-944 HCl) is released within about 7 hours upon administration to a subject. In some embodiments, about 90% of the compound of Formula (I) or a pharmaceutically acceptable salt thereof (e.g., the compound of Formula (II), e.g., PRAX-944 HCl) is released within about 9 hours upon administration to a subject.

In some embodiments, the dosage form comprises from about 1 mg to about 200 mg of the compound of Formula (I) or a pharmaceutically acceptable salt thereof (e.g., the compound of Formula (II), e.g., PRAX-944 HCl) (e.g., from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 105 mg, from about 105 mg to about 110 mg, from about 110 mg to about 115 mg, from about 115 mg to about 120 mg, from about 120 mg to about 125 mg, from about 125 mg to about 130 mg, from about 130 mg to about 135 mg, from about 135 mg to about 140 mg, from about 140 mg to about 145 mg, from about 145 mg to about 150 mg, from about 150 mg to about 155 mg, from about 155 mg to about 160 mg, from about 160 mg to about 165 mg, from about 165 mg to about 170 mg, from about 170 mg to about 175 mg, from about 175 mg to about 180 mg, from about 180 mg to about 185 mg, from about 185 mg to about 190 mg, from about 190 mg to about 195 mg, or from about 195 mg to about 200 mg).

In some embodiments, the dosage form comprises from about 1 mg to about 200 mg of the compound of Formula (I) or a pharmaceutically acceptable salt thereof (e.g., the compound of Formula (II), e.g., PRAX-944 HCl) (e.g., about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg).

In some embodiments, the compositions, formulations, and dosage forms comprising the compound of Formula (I) or a pharmaceutically acceptable salt thereof (e.g., the compound of Formula (II), e.g., PRAX-944 HCl) described herein have a dose strength ranging from about 1 mg to about 200 mg (e.g., from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 105 mg, from about 105 mg to about 110 mg, from about 110 mg to about 115 mg, from about 115 mg to about 120 mg, from about 120 mg to about 125 mg, from about 125 mg to about 130 mg, from about 130 mg to about 135 mg, from about 135 mg to about 140 mg, from about 140 mg to about 145 mg, from about 145 mg to about 150 mg, from about 150 mg to about 155 mg, from about 155 mg to about 160 mg, from about 160 mg to about 165 mg, from about 165 mg to about 170 mg, from about 170 mg to about 175 mg, from about 175 mg to about 180 mg, from about 180 mg to about 185 mg, from about 185 mg to about 190 mg, from about 190 mg to about 195 mg, or from about 195 mg to about 200 mg).

In some embodiments, the compositions, formulations, and dosage forms comprising the compound of Formula (I) or a pharmaceutically acceptable salt thereof (e.g., the compound of Formula (II), e.g., PRAX-944 HCl) described herein have a dose strength ranging from about 1 mg to about 200 mg (e.g., about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg).

In some embodiments, the compositions, formulations, and dosage forms described herein comprises an amount of the compound of Formula (I) or a pharmaceutically acceptable salt thereof (e.g., the compound of Formula (II), e.g., PRAX-944 HCl) equivalent to about 1 mg to about 200 mg (e.g., about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) PRAX-944 free base per dosage unit (e.g., per tablet).

In some embodiments, the single-unit dosage form comprises the compound of Formula (I) or a pharmaceutically acceptable salt thereof (e.g., the compound of Formula (II), e.g., PRAX-944 HCl) in an amount equivalent to about 1.0 mg to about 200 mg (e.g., about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg) of PRAX-944 free base per dosage unit (e.g., per tablet). In some embodiments, the single-unit dosage form comprises from about 1% by weight to about 50% (e.g., about 5%, about 10%, about 15%, about 20%, about 25%, about 30%, about 35%, about 40%, about 45%, or about 50%) by weight of PRAX-944 HCl, optionally wherein the total dosage unit (e.g., tablet) weight is from about 50 mg to about 500 mg (e.g., about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 5 mg of PRAX-944 free base per dosage unit (e.g., per tablet), optionally from about 1 mg to about 10 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 10 mg of PRAX-944 free base per dosage unit (e.g., per tablet), optionally from about 5 mg to about 15 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 15 mg of PRAX-944 free base per dosage unit (e.g., per tablet), optionally from about 10 mg to about 20 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 20 mg of PRAX-944 free base per dosage unit (e.g., per tablet), optionally from about 15 mg to about 25 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 25 mg of PRAX-944 free base per dosage unit (e.g., per tablet), optionally from about 20 mg to about 30 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 30 mg of PRAX-944 free base per dosage unit (e.g., per tablet), optionally from about 25 mg to about 35 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 35 mg of PRAX-944 free base per dosage unit (e.g., per tablet), optionally from about 30 mg to about 40 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 40 mg of PRAX-944 free base per dosage unit (e.g., per tablet), optionally from about 35 mg to about 45 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 45 mg of PRAX-944 free base per dosage unit (e.g., per tablet), optionally from about 40 mg to about 50 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 50 mg of PRAX-944 free base per dosage unit (e.g., per tablet), optionally from about 45 mg to about 55 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 55 mg of PRAX-944 free base per dosage unit (e.g., per tablet), optionally from about 50 mg to about 60 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 60 mg of PRAX-944 free base per dosage unit (e.g., per tablet), optionally from about 55 mg to about 65 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 65 mg of PRAX-944 free base per dosage unit (e.g., per tablet), optionally from about 60 mg to about 70 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 70 mg of PRAX-944 free base per dosage unit (e.g., per tablet), optionally from about 65 mg to about 75 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 75 mg of PRAX-944 free base per dosage unit (e.g., per tablet), optionally from about 70 mg to about 80 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 80 mg of PRAX-944 free base per dosage unit (e.g., per tablet), optionally from about 75 mg to about 85 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 85 mg of PRAX-944 free base per dosage unit (e.g., per tablet), optionally from about 80 mg to about 90 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 90 mg of PRAX-944 free base per dosage unit (e.g., per tablet), optionally from about 85 mg to about 95 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 95 mg of PRAX-944 free base per dosage unit (e.g., per tablet), optionally from about 90 mg to about 100 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 100 mg of PRAX-944 free base per dosage unit (e.g., per tablet), optionally from about 95 mg to about 105 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 105 mg of PRAX-944 free base per dosage unit (e.g., per tablet), optionally from about 100 mg to about 110 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 110 mg of PRAX-944 free base per dosage unit (e.g., per tablet), optionally from about 105 mg to about 115 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 115 mg of PRAX-944 free base per dosage unit (e.g., per tablet), optionally from about 110 mg to about 120 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 120 mg of PRAX-944 free base per dosage unit (e.g., per tablet), optionally from about 115 mg to about 125 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 125 mg of PRAX-944 free base per dosage unit (e.g., per tablet), optionally from about 120 mg to about 130 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 130 mg of PRAX-944 free base per dosage unit (e.g., per tablet), optionally from about 125 mg to about 135 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 135 mg of PRAX-944 free base per dosage unit (e.g., per tablet), optionally from about 130 mg to about 140 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 140 mg of PRAX-944 free base per dosage unit (e.g., per tablet), optionally from about 135 mg to about 145 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 145 mg of PRAX-944 free base per dosage unit (e.g., per tablet), optionally from about 140 mg to about 150 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 150 mg of PRAX-944 free base per dosage unit (e.g., per tablet), optionally from about 145 mg to about 155 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 155 mg of PRAX-944 free base per dosage unit (e.g., per tablet), optionally from about 150 mg to about 160 mg of PRAX-944 HCl per dosage unit (e.g., per tablet).

In some embodiments, the compositions, formulations, and dosage forms described herein comprise the compound of Formula (I) or a pharmaceutically acceptable salt thereof (e.g., the compound of Formula (II), e.g., PRAX-944 HCl) in an amount equivalent to about 160 mg of PRAX-944 free base per dosage unit (e.g., per tablet), optionally from about 155 mg to about 165 mg of PRAX-944 HCl per dosage unit (e.g., per tablet).

In some embodiments, the dosage form comprises from about 1% by weight to about 70% by weight of the compound of Formula (I) or a pharmaceutically acceptable salt thereof (e.g., the compound of Formula (II), e.g., PRAX-944 HCl) (e.g., from about 1% to about 5%, from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, or from about 65% to about 70% by weight of the compound of Formula (I) or a pharmaceutically acceptable salt thereof (e.g., the compound of Formula (II), e.g., PRAX-944 HCl).

In some embodiments, the dosage form comprises from about 1% by weight to about 70% by weight of the compound of Formula (I) or a pharmaceutically acceptable salt thereof (e.g., the compound of Formula (II), e.g., PRAX-944 HCl) (e.g., about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight of the compound of Formula (I) or a pharmaceutically acceptable salt thereof (e.g., the compound of Formula (II), e.g., PRAX-944 HCl).

Exemplary dosage forms comprising the compound of Formula (I) or a pharmaceutically acceptable salt thereof (e.g., the compound of Formula (II), e.g., PRAX-944 HCl) as an active agent are provided in Tables 6-16.

### Modified-Release Polymer

In some embodiments, the dosage form comprises a modified-release polymer.

In some embodiments, the modified-release polymer is used in a formulation (e.g., tablets and capsules) to modify the release rate of the compound of Formula (I) or a pharmaceutically acceptable salt thereof (e.g., the compound of Formula (II), e.g., PRAX-944 HCl) upon the administration to a subject. In some embodiments, the modified-release polymer is used to dissolve the compound of Formula (I) or a pharmaceutically acceptable salt thereof (e.g., the compound of Formula (II), e.g., PRAX-944 HCl) over time in order to be released more slowly, more steadily, or both more slowly and more steadily into the bloodstream.

In some embodiments, the modified-release polymer is a controlled-release polymer. In some embodiments, the modified-release polymer or the controlled-release polymer is a cellulose ether. In some embodiments, the modified-release polymer or the controlled-release polymer is a HPMC (hydroxyl-propyl methylcellulose). In some embodiments, the modified-release polymer or the controlled-release polymer is selected from the group consisting of METHOCEL® E50LV, K100LV, K100LV CR, K4M, K15M, K100M, E4M, E10M, K4MCR, K15MCR, K100MCR, E4MCR, E10MCR, and combinations thereof. In some embodiments, the modified-release polymer or the controlled-release polymer is HPMC K100 LV Premium CR. In some embodiments, the modified-release polymer or the controlled-release polymer is a HPMC (hydroxyl-propyl methylcellulose) selected from the group consisting of Methocel K4M, Methocel K100LV, Methocel E50LV, and combinations thereof. In some embodiments, the modified-release polymer may comprise a matrix polymer (e.g., a hydrophilic matrix polymer, a hydrophobic matrix polymer, a polyacrylate polymer, and combinations thereof). In some embodiments, the modified-release polymer comprises a hydrophilic matrix polymer (e.g., hypromellose, HPMC (hydroxyl-propyl methylcellulose)), a hydrophobic matrix polymer (e.g., ethyl cellulose, ethocel), or a polyacrylate polymer (e.g., Eudragit RL100, Eudragit RS100). In some embodiments, the modified-release polymer is a hydrophilic matrix polymer. In some embodiments, the modified-release polymer is hypromellose. In some embodiments, the modified-release polymer is HPMC (hydroxyl-propyl methylcellulose). In some embodiments, the modified-release polymer is a hydrophobic matrix polymer. In some embodiments, the modified-release polymer is ethyl cellulose. In some embodiments, the modified-release polymer is ethocel. In some embodiments, the modified-release polymer is a polyacrylate polymer. In some embodiments, the modified-release polymer is Eudragit RL100. In some embodiments, the modified-release polymer is Eudragit RS100.

In some embodiments, the dosage form comprises from about 5 mg to 300 mg of a modified-release polymer (e.g., from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 105 mg, from about 105 mg to about 110 mg, from about 110 mg to about 115 mg, from about 115 mg to about 120 mg, from about 120 mg to about 125 mg, from about 125 mg to about 130 mg, from about 130 mg to about 135 mg, from about 135 mg to about 140 mg, from about 140 mg to about 145 mg, from about 145 mg to about 150 mg, from about 150 mg to about 155 mg, from about 155 mg to about 160 mg, from about 160 mg to about 165 mg, from about 165 mg to about 170 mg, from about 170 mg to about 175 mg, from about 175 mg to about 180 mg, from about 180 mg to about 185 mg, from about 185 mg to about 190 mg, from about 190 mg to about 195 mg, from about 195 mg to about 200 mg, from about 200 mg to about 205 mg, from about 205 mg to about 210 mg, from about 210 mg to about 215 mg, from about 215 mg to about 220 mg, from about 220 mg to about 225 mg, from about 225 mg to about 230 mg, from about 230 mg to about 235 mg, from about 235 mg to about 240 mg, from about 240 mg to about 245 mg, from about 245 mg to about 250 mg, from about 250 mg to about 255 mg, from about 255 mg to about 260 mg, from about 260 mg to about 265 mg, from about 265 mg to about 270 mg, from about 270 mg to about 275 mg, from about 275 mg to about 280 mg, from about 280 mg to about 285 mg, from about 285 mg to about 290 mg, from about 290 mg to about 295 mg, or from about 295 mg to about 300 mg of a modified-release polymer).

In some embodiments, the dosage form comprises from about 5 mg to 300 mg of a modified-release polymer (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of a modified-release polymer).

In some embodiments, the dosage form comprises from about 10% by weight to about 70% by weight of the modified-release polymer (e.g., from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, or from about 65% to about 70% by weight of the modified-release polymer).

In some embodiments, the dosage form comprises from about 10% by weight to about 70% by weight of the modified-release polymer (e.g., about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, or about 70% by weight of the modified-release polymer).

In some embodiments, the dosage form comprises from about 5 mg to 300 mg of hypromellose (e.g., from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 105 mg, from about 105 mg to about 110 mg, from about 110 mg to about 115 mg, from about 115 mg to about 120 mg, from about 120 mg to about 125 mg, from about 125 mg to about 130 mg, from about 130 mg to about 135 mg, from about 135 mg to about 140 mg, from about 140 mg to about 145 mg, from about 145 mg to about 150 mg, from about 150 mg to about 155 mg, from about 155 mg to about 160 mg, from about 160 mg to about 165 mg, from about 165 mg to about 170 mg, from about 170 mg to about 175 mg, from about 175 mg to about 180 mg, from about 180 mg to about 185 mg, from about 185 mg to about 190 mg, from about 190 mg to about 195 mg, from about 195 mg to about 200 mg, from about 200 mg to about 205 mg, from about 205 mg to about 210 mg, from about 210 mg to about 215 mg, from about 215 mg to about 220 mg, from about 220 mg to about 225 mg, from about 225 mg to about 230 mg, from about 230 mg to about 235 mg, from about 235 mg to about 240 mg, from about 240 mg to about 245 mg, from about 245 mg to about 250 mg, from about 250 mg to about 255 mg, from about 255 mg to about 260 mg, from about 260 mg to about 265 mg, from about 265 mg to about 270 mg, from about 270 mg to about 275 mg, from about 275 mg to about 280 mg, from about 280 mg to about 285 mg, from about 285 mg to about 290 mg, from about 290 mg to about 295 mg, or from about 295 mg to about 300 mg of hypromellose).

In some embodiments, the dosage form comprises from about 5 mg to 300 mg of hypromellose (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg of hypromellose).

In some embodiments, the dosage form comprises from about 10% by weight to about 70% by weight of hypromellose (e.g., from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, or from about 65% to about 70% by weight of hypromellose).

In some embodiments, the dosage form comprises from about 10% by weight to about 70% by weight of hypromellose (e.g., about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, or about 70% by weight of hypromellose).

### Diluent

In some embodiments, the dosage form comprises a diluent.

In some embodiments, the dosage form comprises a soluble diluent.

In some embodiments, the diluent is used in a formulation (e.g., tablets and capsules) as a filler.

In some embodiments, the term diluent comprises an excipient, e.g., used to increase weight and improve content uniformity. In some embodiments, the diluent may comprise a cellulose derivative (e.g., a microcrystalline cellulose, e.g., a silicified microcrystalline cellulose), a starch (e.g., a hydrolyzed starch, and partially a pregelatinized starch), an anhydrous lactose, a lactose monohydrate, a di-calcium phosphate (DCP), a sugar alcohol (e.g., a sorbitol, a xylitol, and mannitol), and combinations thereof.

In some embodiments, the diluent may comprise PEARLITOL® Mannitol.

In some embodiments, the diluent may comprise mannitol, optionally at a grade selected from the group consisting of MQ, 100SD, 200SD, EZ, XL, M100, M200, 300DC, 400DC, 500DC, 2080, AG, GF, GS, GR, and combinations thereof.

In some embodiments, the dosage form comprises from about 5 mg to about 300 mg of diluent (e.g., from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 105 mg, from about 105 mg to about 110 mg, from about 110 mg to about 115 mg, from about 115 mg to about 120 mg, from about 120 mg to about 125 mg, from about 125 mg to about 130 mg, from about 130 mg to about 135 mg, from about 135 mg to about 140 mg, from about 140 mg to about 145 mg, from about 145 mg to about 150 mg, from about 150 mg to about 155 mg, from about 155 mg to about 160 mg, from about 160 mg to about 165 mg, from about 165 mg to about 170 mg, from about 170 mg to about 175 mg, from about 175 mg to about 180 mg, from about 180 mg to about 185 mg, from about 185 mg to about 190 mg, from about 190 mg to about 195 mg, from about 195 mg to about 200 mg, from about 200 mg to about 205 mg, from about 205 mg to about 210 mg, from about 210 mg to about 215 mg, from about 215 mg to about 220 mg, from about 220 mg to about 225 mg, from about 225 mg to about 230 mg, from about 230 mg to about 235 mg, from about 235 mg to about 240 mg, from about 240 mg to about 245 mg, from about 245 mg to about 250 mg, from about 250 mg to about 255 mg, from about 255 mg to about 260 mg, from about 260 mg to about 265 mg, from about 265 mg to about 270 mg, from about 270 mg to about 275 mg, from about 275 mg to about 280 mg, from about 280 mg to about 285 mg, from about 285 mg to about 290 mg, from about 290 mg to about 295 mg, or from about 295 mg to about 300 mg of diluent).

In some embodiments, the dosage form comprises from about 5 mg to about 300 mg of diluent (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg diluent).

In some embodiments, the dosage form comprises from about 5% by weight to about 50% by weight of diluent (e.g., from about 5% to about 10%, from about 10% to about 15%, from about 15% to about 20%, from about 20% to about 25%, from about 25% to about 30%, from about 30% to about 35%, from about 35% to about 40%, from about 40% to about 45%, or from about 45% to about 50% by weight of diluent).

In some embodiments, the dosage form comprises from about 5% by weight to about 50% by weight of diluent (e.g., about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, about 17% by weight, about 18% by weight, about 19% by weight, about 20% by weight, about 21% by weight, about 22% by weight, about 23% by weight, about 24% by weight, about 25% by weight, about 26% by weight, about 27% by weight, about 28% by weight, about 29% by weight, about 30% by weight, about 31% by weight, about 32% by weight, about 33% by weight, about 34% by weight, about 35% by weight, about 36% by weight, about 37% by weight, about 38% by weight, about 39% by weight, about 40% by weight, about 41% by weight, about 42% by weight, about 43% by weight, about 44% by weight, about 45% by weight, about 46% by weight, about 47% by weight, about 48% by weight, about 49% by weight, or about 50% by weight of diluent).

In some embodiments, the dosage form comprise a microcrystalline cellulose (MCC), e.g., a silicified microcrystalline cellulose (SMCC). MCC and SMCC are commercially available under various trade names (e.g., AVICEL®; EMCOCEL®; MICROCEL®; COMPRECEL®; E460; COELUS KG®; PHARMACEL®; FIBROCEL®, and PROSOLV®), and in different particle sizes, density, and moisture grades.

In some embodiments, the dosage form may comprise a microcrystalline cellulose (MCC) selected from the group consisting of PH 101, PH 102, PH 103, PH 105, PH 112, PH 200, PH 113, PH 301, PH 302, PH 200LM, and combinations thereof.

In some embodiments, the dosage form may comprise a silicified microcrystalline cellulose (SMCC) comprising a microcrystalline cellulose (MCC) and a colloidal silicon dioxide (CSD). In some embodiments, the silicified microcrystalline cellulose comprises from about 90% to about 99% (e.g., about 90%, about 91%, about 92%, about 93%, about 94%, about 95%, about 96%, about 97%, about 98%, or about 99%) microcrystalline cellulose (MCC) by weight, and from about 1% to about 10% (e.g., 1%, about 2%, about 3%, about 4%, about 5%, about 6%, about 7%, about 8%, about 9%, or about 10%) colloidal silicon dioxide (CSD) by weight. In some embodiments, the dosage form may comprise a PROSOLVR SMCC. In some embodiments, the dosage form may comprise a silicified microcrystalline cellulose cellulose (SMCC) selected from the group consisting of PROSOLV® SMCC 50, 50LD, 90, HD 90, 90 LM, and combinations thereof.

In some embodiments, the dosage form may comprise a microcrystalline cellulose (MCC), e.g., a silicified microcrystalline cellulose cellulose (SMCC), having a particle size of less than about 50 μm. In some embodiments, the dosage form may comprise a microcrystalline cellulose (MCC), e.g., a silicified microcrystalline cellulose cellulose (SMCC), having a particle size of greater than about 50 μm. In some embodiments, the dosage form may comprise a microcrystalline cellulose (MCC), e.g., a silicified microcrystalline cellulose cellulose (SMCC), having an average particle size from about 50 μm to about 200 μm (e.g., about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, the dosage form may comprise a microcrystalline cellulose (MCC), e.g., a silicified microcrystalline cellulose cellulose (SMCC), having a moisture content from about 0.1% to about 5% (e.g., about 0.1%, about 0.5%, about 1%, about 1.5%, about 2%, about 2.5%, about 3%, about 3.5%, about 4%, about 4.5%, or about 5%). In some embodiments, the dosage form may comprise a microcrystalline cellulose (MCC), e.g., a silicified microcrystalline cellulose cellulose (SMCC), having a moisture content of less than about 0.5%, less than about 1%, less than about 1.5%, less than about 2%, less than about 2.5%, less than about 3%, less than about 3.5%, less than about 4%, less than about 4.5%, less than about 5%.

In some embodiments, the dosage form comprises from about 5 mg to about 300 mg of microcrystalline cellulose, e.g., silicified microcrystalline cellulose (e.g., from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 105 mg, from about 105 mg to about 110 mg, from about 110 mg to about 115 mg, from about 115 mg to about 120 mg, from about 120 mg to about 125 mg, from about 125 mg to about 130 mg, from about 130 mg to about 135 mg, from about 135 mg to about 140 mg, from about 140 mg to about 145 mg, from about 145 mg to about 150 mg, from about 150 mg to about 155 mg, from about 155 mg to about 160 mg, from about 160 mg to about 165 mg, from about 165 mg to about 170 mg, from about 170 mg to about 175 mg, from about 175 mg to about 180 mg, from about 180 mg to about 185 mg, from about 185 mg to about 190 mg, from about 190 mg to about 195 mg, from about 195 mg to about 200 mg, from about 200 mg to about 205 mg, from about 205 mg to about 210 mg, from about 210 mg to about 215 mg, from about 215 mg to about 220 mg, from about 220 mg to about 225 mg, from about 225 mg to about 230 mg, from about 230 mg to about 235 mg, from about 235 mg to about 240 mg, from about 240 mg to about 245 mg, from about 245 mg to about 250 mg, from about 250 mg to about 255 mg, from about 255 mg to about 260 mg, from about 260 mg to about 265 mg, from about 265 mg to about 270 mg, from about 270 mg to about 275 mg, from about 275 mg to about 280 mg, from about 280 mg to about 285 mg, from about 285 mg to about 290 mg, from about 290 mg to about 295 mg, or from about 295 mg to about 300 mg of microcrystalline cellulose, e.g., silicified microcrystalline cellulose).

In some embodiments, the dosage form comprises from about 5 mg to about 300 mg of microcrystalline cellulose, e.g., silicified microcrystalline cellulose (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg microcrystalline cellulose, e.g., silicified microcrystalline cellulose).

In some embodiments, the dosage form comprises from about 5% by weight to about 50% by weight of microcrystalline cellulose, e.g., silicified microcrystalline cellulose (e.g., from about 5% to about 10%, from about 10% to about 15%, from about 15% to about 20%, from about 20% to about 25%, from about 25% to about 30%, from about 30% to about 35%, from about 35% to about 40%, from about 40% to about 45%, or from about 45% to about 50% by weight of microcrystalline cellulose, e.g., silicified microcrystalline cellulose).

In some embodiments, the dosage form comprises from about 5% by weight to about 50% by weight of microcrystalline cellulose, e.g., silicified microcrystalline cellulose (e.g., about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, about 17% by weight, about 18% by weight, about 19% by weight, about 20% by weight, about 21% by weight, about 22% by weight, about 23% by weight, about 24% by weight, about 25% by weight, about 26% by weight, about 27% by weight, about 28% by weight, about 29% by weight, about 30% by weight, about 31% by weight, about 32% by weight, about 33% by weight, about 34% by weight, about 35% by weight, about 36% by weight, about 37% by weight, about 38% by weight, about 39% by weight, about 40% by weight, about 41% by weight, about 42% by weight, about 43% by weight, about 44% by weight, about 45% by weight, about 46% by weight, about 47% by weight, about 48% by weight, about 49% by weight, or about 50% by weight of microcrystalline cellulose, e.g., silicified microcrystalline cellulose).

In some embodiments, the dosage form comprises from about 5 mg to about 300 mg of sugar alcohol, e.g., mannitol (e.g., from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 105 mg, from about 105 mg to about 110 mg, from about 110 mg to about 115 mg, from about 115 mg to about 120 mg, from about 120 mg to about 125 mg, from about 125 mg to about 130 mg, from about 130 mg to about 135 mg, from about 135 mg to about 140 mg, from about 140 mg to about 145 mg, from about 145 mg to about 150 mg, from about 150 mg to about 155 mg, from about 155 mg to about 160 mg, from about 160 mg to about 165 mg, from about 165 mg to about 170 mg, from about 170 mg to about 175 mg, from about 175 mg to about 180 mg, from about 180 mg to about 185 mg, from about 185 mg to about 190 mg, from about 190 mg to about 195 mg, from about 195 mg to about 200 mg, from about 200 mg to about 205 mg, from about 205 mg to about 210 mg, from about 210 mg to about 215 mg, from about 215 mg to about 220 mg, from about 220 mg to about 225 mg, from about 225 mg to about 230 mg, from about 230 mg to about 235 mg, from about 235 mg to about 240 mg, from about 240 mg to about 245 mg, from about 245 mg to about 250 mg, from about 250 mg to about 255 mg, from about 255 mg to about 260 mg, from about 260 mg to about 265 mg, from about 265 mg to about 270 mg, from about 270 mg to about 275 mg, from about 275 mg to about 280 mg, from about 280 mg to about 285 mg, from about 285 mg to about 290 mg, from about 290 mg to about 295 mg, or from about 295 mg to about 300 mg of sugar alcohol, e.g., mannitol).

In some embodiments, the dosage form comprises from about 5 mg to about 300 mg of sugar alcohol, e.g., mannitol (e.g., about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, or about 300 mg sugar alcohol, e.g., mannitol).

In some embodiments, the dosage form comprises from about 5% by weight to about 50% by weight of sugar alcohol, e.g., mannitol (e.g., from about 5% to about 10%, from about 10% to about 15%, from about 15% to about 20%, from about 20% to about 25%, from about 25% to about 30%, from about 30% to about 35%, from about 35% to about 40%, from about 40% to about 45%, or from about 45% to about 50% by weight of sugar alcohol, e.g., mannitol).

In some embodiments, the dosage form comprises from about 5% by weight to about 50% by weight of sugar alcohol, e.g., mannitol (e.g., about 5% by weight, about 6% by weight, about 7% by weight, about 8% by weight, about 9% by weight, about 10% by weight, about 11% by weight, about 12% by weight, about 13% by weight, about 14% by weight, about 15% by weight, about 16% by weight, about 17% by weight, about 18% by weight, about 19% by weight, about 20% by weight, about 21% by weight, about 22% by weight, about 23% by weight, about 24% by weight, about 25% by weight, about 26% by weight, about 27% by weight, about 28% by weight, about 29% by weight, about 30% by weight, about 31% by weight, about 32% by weight, about 33% by weight, about 34% by weight, about 35% by weight, about 36% by weight, about 37% by weight, about 38% by weight, about 39% by weight, about 40% by weight, about 41% by weight, about 42% by weight, about 43% by weight, about 44% by weight, about 45% by weight, about 46% by weight, about 47% by weight, about 48% by weight, about 49% by weight, or about 50% by weight of sugar alcohol, e.g., mannitol).

### Glidant

In some embodiments, the dosage form comprises a glidant.

In some embodiments, the glidant comprises an excipient, e.g., used to promote powder flow by reducing interparticle friction and cohesion. In some embodiments, the glidant comprises fumed silica (e.g., colloidal silicon dioxide), talc, and magnesium carbonate. In some embodiments, the glidant comprises colloidal silicon dioxide. In some embodiments, the colloidal silicon dioxide may comprise AEROSIL® 200 Pharma, also referred to as “colloidal silicon dioxide (Aerosil 200).”

In some embodiments, the dosage form comprises from about 1 mg to about 10 mg of glidant (e.g., from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, or from about 9 mg to about 10 mg of glidant).

In some embodiments, the dosage form comprises from about 1 mg to about 10 mg of glidant (e.g., about 1 mg, about 2 mg, about 3 mg, about 4 mg, about 5 mg, about 6 mg, about 7 mg, about 8 mg, about 9 mg, or about 10 mg glidant).

In some embodiments, the dosage form comprises from about 1% by weight to about 10% by weight of glidant (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, or from about 9% to about 10% by weight of glidant).

In some embodiments, the dosage form comprises from about 1% by weight to about 10% by weight of glidant (e.g., about 1% by weight, about 2% by weight, about 3% by weight, about 4% by weight, about 5% by weight, about 6% by weight, about 7% by weight, about 8% by weight, about 9% by weight, or about 10% by weight of glidant).

In some embodiments, the dosage form comprises from about 1 mg to about 10 mg of fumed silica, e.g., colloidal silicon dioxide (e.g., from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, or from about 9 mg to about 10 mg of fumed silica, e.g., colloidal silicon dioxide).

In some embodiments, the dosage form comprises from about 1 mg to about 10 mg of fumed silica, e.g., colloidal silicon dioxide (e.g., about 1 mg, about 2 mg, about 3 mg, about 4 mg, about 5 mg, about 6 mg, about 7 mg, about 8 mg, about 9 mg, or about 10 mg fumed silica, e.g., colloidal silicon dioxide).

In some embodiments, the dosage form comprises from about 1% by weight to about 10% by weight of fumed silica, e.g., colloidal silicon dioxide (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, or from about 9% to about 10% by weight of fumed silica, e.g., colloidal silicon dioxide).

In some embodiments, the dosage form comprises from about 1% by weight to about 10% by weight of fumed silica, e.g., colloidal silicon dioxide (e.g., about 1% by weight, about 2% by weight, about 3% by weight, about 4% by weight, about 5% by weight, about 6% by weight, about 7% by weight, about 8% by weight, about 9% by weight, or about 10% by weight of fumed silica, e.g., colloidal silicon dioxide).

### Lubricant

In some embodiments, the composition comprises a lubricant.

In some embodiments, the lubricant comprises an excipient, e.g., used to prevent ingredients from clumping together and from sticking to the tablet punches or capsule filling machine. In some embodiments, lubricants are also used to ensure that tablet formation and ejection can occur with low friction between the solid and die wall. In some embodiments, lubricants comprise magnesium stearate, calcium stearate, stearic acid, talc, silica, and fats (e.g., vegetable stearin). In some embodiments, the lubricant comprises magnesium stearate.

In some embodiments, the dosage form comprises from about 1 mg to about 10 mg of lubricant (e.g., from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, or from about 9 mg to about 10 mg of lubricant).

In some embodiments, the dosage form comprises from about 1 mg to about 10 mg of lubricant (e.g., about 1 mg, about 2 mg, about 3 mg, about 4 mg, about 5 mg, about 6 mg, about 7 mg, about 8 mg, about 9 mg, or about 10 mg lubricant).

In some embodiments, the dosage form comprises from about 1% by weight to about 10% by weight of lubricant (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, or from about 9% to about 10% by weight of lubricant).

In some embodiments, the dosage form comprises from about 1% by weight to about 10% by weight of lubricant (e.g., about 1% by weight, about 2% by weight, about 3% by weight, about 4% by weight, about 5% by weight, about 6% by weight, about 7% by weight, about 8% by weight, about 9% by weight, or about 10% by weight of lubricant).

In some embodiments, the dosage form comprises from about 1 mg to about 10 mg of magnesium stearate (e.g., from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, or from about 9 mg to about 10 mg of magnesium stearate).

In some embodiments, the dosage form comprises from about 1 mg to about 10 mg of magnesium stearate (e.g., about 1 mg, about 2 mg, about 3 mg, about 4 mg, about 5 mg, about 6 mg, about 7 mg, about 8 mg, about 9 mg, or about 10 mg magnesium stearate).

In some embodiments, the dosage form comprises from about 1% by weight to about 10% by weight of magnesium stearate (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, or from about 9% to about 10% by weight of magnesium stearate).

In some embodiments, the dosage form comprises from about 1% by weight to about 10% by weight of magnesium stearate (e.g., about 1% by weight, about 2% by weight, about 3% by weight, about 4% by weight, about 5% by weight, about 6% by weight, about 7% by weight, about 8% by weight, about 9% by weight, or about 10% by weight of magnesium stearate).

### Coating

In some embodiments, the composition comprises a coating.

In some embodiments, the coating comprises an excipient, e.g., to protect tablet ingredients from deterioration by moisture in the air and make large or unpleasant-tasting tablets easier to swallow. In some embodiments, the coating comprises a film coating agent. In some embodiments, the coating comprises Opadry® II white 85F18422, which is comprised of compendial grade polyvinyl alcohol, titanium dioxide, polyethylene glycol 3350, and talc.

In some embodiments, the dosage form comprises from about 1 mg to about 20 mg of coating, e.g., film coating agent (e.g., from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, from about 9 mg to about 10 mg, from about 10 mg to about 11 mg, from about 11 mg to about 12 mg, from about 12 mg to about 13 mg, from about 13 mg to about 14 mg, from about 14 mg to about 15 mg, from about 15 mg to about 16 mg, from about 16 mg to about 17 mg, from about 17 mg to about 18 mg, from about 18 mg to about 19 mg, or from about 19 mg to about 20 mg of coating, e.g., film coating agent).

In some embodiments, the dosage form comprises from about 1 mg to about 20 mg of coating, e.g., film coating agent (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg coating, e.g., film coating agent).

In some embodiments, the dosage form comprises from about 1% by weight to about 10% by weight of coating, e.g., film coating agent (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10% by weight of coating, e.g., film coating agent).

In some embodiments, the dosage form comprises from about 1% by weight to about 10% by weight of coating, e.g., film coating agent (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10% about 1% by weight, about 2% by weight, about 3% by weight, about 4% by weight, about 5% by weight, about 6% by weight, about 7% by weight, about 8% by weight, about 9% by weight, or about 10% by weight of coating, e.g., film coating agent).

In some embodiments, the dosage form comprises from about 1 mg to about 20 mg of Opadry® II white 85F18422 (e.g., from about 1 mg to about 2 mg, from about 2 mg to about 3 mg, from about 3 mg to about 4 mg, from about 4 mg to about 5 mg, from about 5 mg to about 6 mg, from about 6 mg to about 7 mg, from about 7 mg to about 8 mg, from about 8 mg to about 9 mg, from about 9 mg to about 10 mg, from about 10 mg to about 11 mg, from about 11 mg to about 12 mg, from about 12 mg to about 13 mg, from about 13 mg to about 14 mg, from about 14 mg to about 15 mg, from about 15 mg to about 16 mg, from about 16 mg to about 17 mg, from about 17 mg to about 18 mg, from about 18 mg to about 19 mg, or from about 19 mg to about 20 mg of Opadry® II white 85F18422).

In some embodiments, the dosage form comprises from about 1 mg to about 20 mg of Opadry® II white 85F18422 (e.g., about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg Opadry® II white 85F18422).

In some embodiments, the dosage form comprises from about 1% by weight to about 10% by weight of Opadry® II white 85F18422 (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10% by weight of Opadry® II white 85F18422).

In some embodiments, the dosage form comprises from about 1% by weight to about 10% by weight of Opadry® II white 85F18422 (e.g., from about 1% to about 2%, from about 2% to about 3%, from about 3% to about 4%, from about 4% to about 5%, from about 5% to about 6%, from about 6% to about 7%, from about 7% to about 8%, from about 8% to about 9%, from about 9% to about 10% about 1% by weight, about 2% by weight, about 3% by weight, about 4% by weight, about 5% by weight, about 6% by weight, about 7% by weight, about 8% by weight, about 9% by weight, or about 10% by weight of Opadry® II white 85F18422).

### Form Factor

The compositions, formulations, and dosage forms described herein may be manufactured using any appropriate manufacturing method known in the art including, but not limited to, direct compression, dry granulation (e.g., slugging or roller compression), wet granulation, and any combination thereof. The compositions, formulations, and dosage forms described herein should advantageously be of a shape, size, and weight that can be taken, e.g., orally, with ease. Accordingly, in one aspect, the compositions, formulations, and dosage forms described herein may be a solid dosage form suitable for oral administration. The dosage form may be selected from the group consisting of tablets, capsules, films, powders, granules, solutions, solids, suspensions and another acceptable oral dosage forms. In some embodiments, the dosage form is a tablet, such as a caplet. In other embodiments, the dosage form is a capsule. In certain embodiments, the dosage form is a suspension.

In some embodiments, the dosage form (e.g., tablet) may be compressed or molded in its manufacture, and it may be of almost any size, shape, weight, and color. In some embodiments, the dosage form (e.g., tablet) is in the shape of a capsule (also referred to as a “caplet”). Most tablets are intended to be swallowed whole, and accordingly, preferred tablets are designed for oral administration. However, in some embodiments, tablets may be dissolved in the mouth, chewed, or dissolved in liquid before swallowing, and some may be placed in a body cavity.

In some embodiments, the total dosage form (e.g., tablet) weight, which is a function of the total size of the dosage form (e.g., tablet), can be adjusted in order to provide the best compromise between desired pharmacokinetics (PK) and patient compliance.

In some embodiments, the dosage form (e.g., tablet) has a total weight from about 50 mg to about 600 mg (e.g., from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 275 mg, from about 275 mg to about 300 mg, from about 300 mg to about 325 mg, from about 325 mg to about 350 mg, from about 350 mg to about 375 mg, from about 375 mg to about 400 mg, from about 400 mg to about 425 mg, from about 425 mg to about 450 mg, from about 450 mg to about 475 mg, from about 475 mg to about 500 mg, from about 500 mg to about 525 mg, from about 525 mg to about 550 mg, from about 550 mg to about 575 mg, or from about 575 mg to about 600 mg).

In some embodiments, the dosage form (e.g., tablet) has a total weight from about 50 mg to about 600 mg (e.g., about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, or about 600 mg).

In some embodiments, the dosage form is a round tablet. In some embodiments, the tablet may have a diameter in the range of about 1 mm to about 30 mm (e.g., about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, or about 30 mm). In some embodiments, the tablet does not have a diameter in the range of about 1 mm to about 6 mm (e.g., about 1 mm, about 2 mm, about 3 mm, about 4 mm, about 5 mm, about 6 mm).

In some embodiments, the dosage form is an oblong tablet. In some embodiments, opening blister packages of oblong tablets, handling oblong tablets, and taking one or more oblong tablets is less challenging and move convenient for patients struggling with movement disorders, such as essential tremor (ET), e.g., as compared to a small, round tablet (e.g., 6 mm in diameter).

In some embodiments, the dosage form is an oval tablet. In some embodiments, opening blister packages of oval tablets, handling oval tablets, and taking one or more oval tablets is less challenging and move convenient for patients struggling with movement disorders, such as essential tremor (ET), e.g., as compared to a small, round tablet (e.g., 6 mm in diameter).

In some embodiments, the dosage form is a capsule-shaped tablet (e.g., a caplet). In some embodiments, opening blister packages of capsule-shaped tablets, handling capsule-shaped tablets, and taking one or more capsule-shaped tablets is less challenging and move convenient for patients struggling with movement disorders, such as essential tremor (ET), e.g., as compared to a small, round tablet (e.g., 6 mm in diameter).

In some embodiments, the dosage form (e.g., tablet) may have a length of about 1 mm to about 30 mm (e.g., from about 1 mm to about 2 mm, from about 2 mm to about 3 mm, from about 3 mm to about 4 mm, from about 4 mm to about 5 mm, from about 5 mm to about 6 mm, from about 6 mm to about 7 mm, from about 7 mm to about 8 mm, from about 8 mm to about 9 mm, from about 9 mm to about 10 mm, from about 10 mm to about 11 mm, from about 11 mm to about 12 mm, from about 12 mm to about 13 mm, from about 13 mm to about 14 mm, from about 14 mm to about 15 mm, from about 15 mm to about 16 mm, from about 16 mm to about 17 mm, from about 17 mm to about 18 mm, from about 18 mm to about 19 mm, from about 19 mm to about 20 mm, from about 20 mm to about 21 mm, from about 21 mm to about 22 mm, from about 22 mm to about 23 mm, from about 23 mm to about 24 mm, from about 24 mm to about 25 mm, from about 25 mm to about 26 mm, from about 26 mm to about 27 mm, from about 27 mm to about 28 mm, from about 28 mm to about 29 mm, or from about 29 mm to about 30 mm).

In some embodiments, the dosage form (e.g., tablet) may have a length of about 1 mm to about 30 mm (e.g., about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, about 20.5 mm, about 21 mm, about 21.5 mm, about 22 mm, about 22.5 mm, about 23 mm, about 23.5 mm, about 24 mm, about 24.5 mm, about 25 mm, about 25.5 mm, about 26 mm, about 26.5 mm, about 27 mm, about 27.5 mm, about 28 mm, about 28.5 mm, about 29 mm, about 29.5 mm, or about 30 mm).

In some embodiments, the dosage form (e.g., tablet) may have a width of about 1 mm to about 30 mm (e.g., from about 1 mm to about 2 mm, from about 2 mm to about 3 mm, from about 3 mm to about 4 mm, from about 4 mm to about 5 mm, from about 5 mm to about 6 mm, from about 6 mm to about 7 mm, from about 7 mm to about 8 mm, from about 8 mm to about 9 mm, from about 9 mm to about 10 mm, from about 10 mm to about 11 mm, from about 11 mm to about 12 mm, from about 12 mm to about 13 mm, from about 13 mm to about 14 mm, from about 14 mm to about 15 mm, from about 15 mm to about 16 mm, from about 16 mm to about 17 mm, from about 17 mm to about 18 mm, from about 18 mm to about 19 mm, from about 19 mm to about 20 mm, from about 20 mm to about 21 mm, from about 21 mm to about 22 mm, from about 22 mm to about 23 mm, from about 23 mm to about 24 mm, from about 24 mm to about 25 mm, from about 25 mm to about 26 mm, from about 26 mm to about 27 mm, from about 27 mm to about 28 mm, from about 28 mm to about 29 mm, or from about 29 mm to about 30 mm).

In some embodiments, the dosage form (e.g., tablet) may have a width of about 1 mm to about 30 mm (e.g., about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, about 20 mm, about 20.5 mm, about 21 mm, about 21.5 mm, about 22 mm, about 22.5 mm, about 23 mm, about 23.5 mm, about 24 mm, about 24.5 mm, about 25 mm, about 25.5 mm, about 26 mm, about 26.5 mm, about 27 mm, about 27.5 mm, about 28 mm, about 28.5 mm, about 29 mm, about 29.5 mm, or about 30 mm).

In some embodiments, the dosage form (e.g., tablet) may have a width of about 1 mm to about 10 mm (e.g., from about 1 mm to about 2 mm, from about 2 mm to about 3 mm, from about 3 mm to about 4 mm, from about 4 mm to about 5 mm, from about 5 mm to about 6 mm, from about 6 mm to about 7 mm, from about 7 mm to about 8 mm, from about 8 mm to about 9 mm, or from about 9 mm to about 10 mm).

In some embodiments, the dosage form (e.g., tablet) comprises a width of about 1 mm to about 10 mm (e.g., a width of about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, or about 10 mm).

In some embodiments, the dosage form (e.g., tablet) may have a width of about 1 mm to about 10 mm (e.g., about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm).

In some embodiments, the dosage form (e.g., tablet) may have a length of about 10 to about 20 mm (e.g., about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, or about 20 mm) and a width of about 1 mm to about 10 mm (e.g., about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm).

In some embodiments, the dosage form (e.g., tablet) may have a length of about 14 to about 16 mm (e.g., about 14 mm, about 14.1 mm, about 14.2 mm, about 14.3 mm, about 14.4 mm, about 14.5 mm, about 14.6 mm, about 14.7 mm, about 14.8 mm, about 14.9 mm, about 15 mm, about 15.1 mm, about 15.2 mm, about 15.3 mm, about 15.4 mm, about 15.5 mm, about 15.6 mm, about 15.7 mm, about 15.8 mm, about 15.9 mm, or about 16 mm) and a width of about 5 mm to about 7 mm (e.g., 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm).

In some embodiments, the dosage form (e.g., tablet) may have a thickness of about 1 mm to about 10 mm (e.g., about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, or about 10 mm).

Tablet hardness can be measured using any technique or apparatus known in the art for testing tablet hardness. For example, a force gauge can be used to determine breaking force, which is indicative of the strength of the tablet. Typical hardness measurement units include: kiloponds (kp), Strong Cobb Units (SCU), and Newtons (N). In some embodiments, the dosage form (e.g., tablet) may have a hardness of about 75 N, about 76 N, about 77 N, about 78 N, about 79 N, about 80 N, about 81 N, about 82 N, about 83 N, about 84 N, about 85 N, about 86 N, about 87 N, about 88 N, about 89 N, about 90 N, about 91 N, about 92 N, about 93 N, about 94 N, about 95 N, about 96 N, about 97 N, about 98 N, about 99 N, about 100 N, about 101 N, about 102 N, about 103 N, about 104 N, about 105 N, about 106 N, about 107 N, about 108 N, about 109 N, about 110 N, about 111 N, about 112 N, about 113 N, about 114 N, about 115 N, about 116 N, about 117 N, about 118 N, about 119 N, about 120 N, about 121 N, about 122 N, about 123 N, about 124 N, about 125 N, about 126 N, about 127 N, about 128 N, about 129 N, about 130 N, about 131 N, about 132 N, about 133 N, about 134 N, about 135 N, about 136 N, about 137 N, about 138 N, about 139 N, about 140 N, about 141 N, about 142 N, about 143 N, about 144 N, about 145 N, about 146 N, about 147 N, about 148 N, about 149 N, about 150 N, about 151 N, about 152 N, about 153 N, about 154 N, about 155 N, about 156 N, about 157 N, about 158 N, about 159 N, about 160 N, about 161 N, about 162 N, about 163 N, about 164 N, about 165 N, about 166 N, about 167 N, about 168 N, about 169 N, about 170 N, about 171 N, about 172 N, about 173 N, about 174 N, about 175 N, about 176 N, about 177 N, about 178 N, about 179 N, about 180 N, about 181 N, about 182 N, about 183 N, about 184 N, about 185 N, about 186 N, about 187 N, about 188 N, about 189 N, about 190 N, about 191 N, about 192 N, about 193 N, about 194 N, about 195 N, about 196 N, about 197 N, about 198 N, about 199 N, or about 200 N). In some embodiments, the dosage form (e.g., tablet) may have a hardness of about 90 N to about 110 N. In some embodiments, the dosage form (e.g., tablet) may have a hardness of about 100 N to about 120 N. In some embodiments, the dosage form (e.g., tablet) may have a hardness of about 110 N to about 130 N. In some embodiments, the dosage form (e.g., tablet) may have a hardness of about 120 N to about 140 N. In some embodiments, the dosage form (e.g., tablet) may have a hardness of about 130 N to about 150 N. In some embodiments, the dosage form (e.g., tablet) may have a hardness of about 140 N to about 160 N. In some embodiments, the dosage form (e.g., tablet) may have a hardness of about 150 N to about 170 N. In some embodiments, the dosage form (e.g., tablet) may have a hardness of about 160 N to about 180 N. In some embodiments, the dosage form (e.g., tablet) may have a hardness of about 170 N to about 190 N. In some embodiments, the dosage form (e.g., tablet) may have a hardness of about 180 N to about 200 N.

In some embodiments, the dosage form (e.g., tablet) may have a compression force at observed hardness of about 5 N to about 35 N (e.g., about 5 N, about 6 N, about 7 N, about 8 N, about 9 N, about 10 N, about 11 N, about 12 N, about 13 N, about 14 N, about 15 N, about 16 N, about 17 N, about 18 N, about 19 N, about 20 N, about 21 N, about 22 N, about 23 N, about 24 N, about 25 N, about 26 N, about 27 N, about 28 N, about 29 N, about 30 N, about 31 N, about 32 N, about 33 N, about 34 N, or about 35 N). In some embodiments, the dosage form (e.g., tablet) may have a compression force at observed hardness of about 5 N to about 15 N. In some embodiments, the dosage form (e.g., tablet) may have a compression force at observed hardness of about 10 N to about 20 N. In some embodiments, the dosage form (e.g., tablet) may have a compression force at observed hardness of about 15 N to about 25 N. In some embodiments, the dosage form (e.g., tablet) may have a compression force at observed hardness of about 20 N to about 30 N. In some embodiments, the dosage form (e.g., tablet) may have a compression force at observed hardness of about 25 N to about 35 N. In some embodiments, the dosage form (e.g., tablet) may have a compression force at observed hardness of about 30 N to about 35 N.

In some embodiments, the dosage form comprises an amount of the compound of Formula (I) or a pharmaceutically acceptable salt thereof (e.g., the compound of Formula (II)) equivalent to about 5.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 10.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 20.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 30.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 40.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 50.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 60.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 70.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 80.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 90.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 100.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 110.0 mg PRAX-944 free base per dosage unit (e.g., per tablet).

In some embodiments, the dosage form comprises an amount of the compound of Formula (I) or a pharmaceutically acceptable salt thereof (e.g., the compound of Formula (II)) equivalent to about 120.0 mg PRAX-944 free base per dosage unit (e.g., per tablet).

### Pharmaceutical Compositions

The present disclosure encompasses the preparation and use of pharmaceutical compositions comprising the compound of Formula (I) or a pharmaceutically acceptable salt thereof (e.g., the compound of Formula (II), e.g., PRAX-944 HCl) as an active agent. Such a pharmaceutical composition may consist of the active agent alone, as a combination of at least one active agent (e.g., an effective dose of the compound of Formula (I) or a pharmaceutically acceptable salt thereof (e.g., the compound of Formula (II), e.g., PRAX-944 HCl)) in a form suitable for administration to a subject, or the pharmaceutical composition may comprise the active agent and one or more pharmaceutically acceptable carriers, one or more additional (active and/or inactive) ingredients, or some combination thereof.

In one aspect, the pharmaceutical compositions provided by the present disclosure include a single-unit dosage form comprising the compound of Formula (I) or a pharmaceutically acceptable salt thereof (e.g., the compound of Formula (II)). In certain embodiments, single-unit dosage form comprises up to 200 mg of the compound of Formula (I) or a pharmaceutically acceptable salt thereof. In some embodiments, the single-unit dosage form comprises a length of up to 16 mm, e.g., about 14 mm to about 16 mm, and/or a width of up to 7 mm, e.g., about 5 mm to about 7 mm. In some embodiments, the single-unit dosage form is bioequivalent to a reference composition of the same dosage strength administered as multiple dosage forms, such as the a modified release (MR) formulation comprising the compound of Formula (I) available as 5-mg and 20-mg tablets that are round and small in size (e.g., about 6 mm in diameter). In some embodiments, bioequivalency may be established by: (a) a 90% Confidence Interval for AUC which is between about 80% and about 125%, and (b) a 90% Confidence Interval for Cmax, which is between about 80% and about 125%.

Exemplary pharmaceutical compositions comprising the compound of Formula (I) or a pharmaceutically acceptable salt thereof (e.g., the compound of Formula (II), e.g., PRAX-944 HCl) as an active agent are provided in Tables 6-16.

The pharmaceutical compositions of the disclosure may contain a therapeutically effective amount of the compound of Formula (I) or a pharmaceutically acceptable salt thereof (e.g., the compound of Formula (II), e.g., PRAX-944 HCl). Those skilled in the art will recognize, however, that a pharmaceutical composition may contain more than a therapeutically effective amount, such as in bulk compositions, or less than a therapeutically effective amount, that is, individual unit doses designed for multiple administration to achieve a therapeutically effective amount. Typically, the composition will contain from about 1 mg to about 200 mg of the compound of Formula (I) or a pharmaceutically acceptable salt thereof (e.g., the compound of Formula (II), e.g., PRAX-944 HCl), with the actual amount depending upon the formulation itself, the route of administration, the frequency of dosing, and so forth. According to one embodiment, a composition suitable for an oral dosage form, for example, may contain about 5 mg, about 10 mg, about 20 mg, about 40 mg, about 60 mg, about 80 mg, about 100 mg, or about 120 mg of the compound of Formula (I) or a pharmaceutically acceptable salt thereof (e.g., the compound of Formula (II), e.g., PRAX-944 HCl).

The pharmaceutical compositions of the present disclosure are designed to provide a larger, more convenient PRAX-944 dosage form as compared to the currently available PRAX-944 dosage form, which is a modified release (MR) formulation available as 5-mg and 20-mg tablets that are round and small in size (6 mm in diameter). For example, the pharmaceutical compositions of the present disclosure are designed to minimize the total number of dosage units (e.g., tablets) needed to deliver specific dose levels of PRAX-944 and to increase the physical size of the tablet to make it easier to grasp and more convenient for patients, especially those with a movement disorder, such as for example essential tremor (ET). Further, the pharmaceutical compositions of the present disclosure comprising larger tablets have substantially the same bioavailability, e.g., with respect to the rate (maximum plasma drug concentration; Cmax) and extent (area under the plasma concentration-time curve; AUC) of absorption of PRAX-944 compared to multiple small, round 20-mg tablets of the same total dosage.

Pharmaceutical compositions of the present disclosure may be administered in a manner appropriate to the condition, disease, and/or disorder to be treated (or prevented). The quantity and frequency of administration will be determined by such factors as the condition of the patient, and the type and severity of the patient's condition, disease, and/or disorder, although appropriate dosages may be determined by clinical trials. The administration of the pharmaceutical compositions may be carried out in any convenient manner, including, e.g., by oral administration.

As used herein, the term “pharmaceutically acceptable carrier” means a chemical composition with which the active agent may be combined and which, following the combination, can be used to administer the active agent to a subject. Suitable carriers are described in the most recent edition of Remington's Pharmaceutical Sciences, a standard reference text in the field, which is incorporated herein by reference.

The formulations of the pharmaceutical compositions described herein may be prepared by any method known or hereafter developed in the art of pharmacology. In general, such preparatory methods include the step of bringing the active agent into association with a carrier or one or more other accessory ingredients, and then, if necessary or desirable, shaping or packaging the product into a desired single- or multi-dose unit.

### Kits

In one aspect, one or more dosage forms as described herein (e.g., one or more 5 mg tablets, one or more 10 mg tablets, one or more 20 mg tablets, one or more 40 mg tablets, one or more 80 mg tablets, and/or one or more 120 mg tablets) may be provided, e.g., in packaging, such as kits, blister packs, packets, or bottles shrink-wrapped together. In one embodiment, a kit is provided which contains a plurality of oral dosage forms in packaging together, and instructions for use to administer the oral dosage forms in accordance with the method described herein. The oral dosage form may be selected from the group consisting of tablets, capsules, films, powders, granules, solutions, solids, and suspensions.

The packaged oral dosage forms can contain a fill supply of the medication typically prescribed for the intended therapy, e.g., titration regimen. A series of unit doses can be packaged together in accordance with the prescribed regimen or treatment, e.g., a 1-90 day supply, depending on the particular therapy. In some embodiments, a series of unit doses can comprise one or more 5 mg tablets, one or more 10 mg tablets, one or more 20 mg tablets, one or more 40 mg tablets, one or more 80 mg tablets, and/or one or more 120 mg tablets as described herein. In some embodiments, up to about a 90-day supply of each dosage amount from about 5 mg to about 200 mg (e.g., one or more 5 mg tablets, one or more 10 mg tablets, one or more 20 mg tablets, one or more 40 mg tablets, one or more 80 mg tablets, and/or one or more 120 mg tablets as described herein) may be provided, e.g., in packaging, together.

In one embodiment, oral dosage forms, e.g., tablets, as described herein may be included in a blister pack with instructions to administer one or more tablets on a daily basis so that the dosage of the formulations described herein are sufficiently administered. In one embodiment, oral dosage forms, e.g., tablets, as described herein may be included in a blister pack with instructions to administer two or more tablets on a daily basis so that the dosage of the formulations described herein are sufficiently administered. In another embodiment, oral dosage forms are included in a blister pack with instructions to administer one or more tablets on an alternate day basis so that the dosage per day is sufficiently administered. In one embodiment, oral dosage forms, e.g., tablets, as described herein may be included in a blister pack with instructions to administer one or more tablets on a weekly basis so that the dosage of the formulations described herein are sufficiently administered.

### Methods of Treating a Disease or Condition Relating to Aberrant Function or Activity of a T-Type Calcium Channel

In one aspect, the present disclosure provides a method of treating a disease or condition relating to aberrant function or activity of a T-type calcium channel, such as essential tremor (ET), in a subject in need thereof, the method comprising administering (e.g., once, twice, three times) daily to the subject a therapeutically effective amount of the compound of Formula (I):

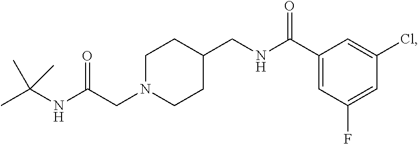 [SMILES: CC(C)(C)NC(=O)CN1CCC(CNC(=O)c2cc(F)cc(Cl)c2)CC1]

or a pharmaceutically acceptable salt (e.g., co-crystal) or solvate thereof, for example, a compound of Formula (II):

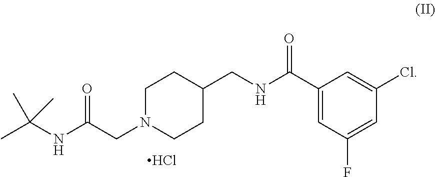 [SMILES: CC(C)(C)NC(=O)CN1CCC(CNC(=O)c2cc(F)cc(Cl)c2)CC1 ; Cl]

Formula (I) may also be referred to as N-((1-(2-(tert-butylamino)-2-oxoethyl)piperidin-4-yl)methyl)-3-chloro-5-fluorobenzamide, while Formula (II) may be referred to as N-((1-(2-(tert-butylamino)-2-oxoethyl)piperidin-4-yl)methyl)-3-chloro-5-fluorobenzamide hydrochloride.

In some embodiments, methods of the present disclosure comprise administering to a subject in need thereof a single-unit dosage form, comprising the compound of Formula (I) or a pharmaceutically acceptable salt thereof (e.g., the compound of Formula (II)), wherein the composition is bioequivalent to a reference composition of the same dosage strength administered as multiple smaller, round tablets.

In some embodiments, methods of the present disclosure comprise administering to a subject in need thereof a titrated dose of the compound of formula (I) or (II). In some embodiments, the maximum titrated dose is 60 mg per day or 100 mg per day. In one embodiment, the maximum titrated dose is 60 mg per day.

Disclosed herein are methods of treating a disease or condition relating to aberrant function or activity of a T-type calcium channel, comprising administering a titrated dose such that the end or maintenance dosage exceeds an initial dosage or a dosage at which adverse events are likely to be experienced absent titration (a maximum tolerated dosage achieved without titration). As used herein, administering a titrated dose refers to the practice of beginning with a low dosage and escalating to one or more higher dosages. For example, in certain embodiments, administering a titrated dose according to a method described herein may comprise: (i) administering a first dose, such as a dose of about 5 mg daily of a compound of Formula (I) or a pharmaceutically acceptable salt thereof to the subject for a first period of time (e.g., the first week); (ii) administering a second dose, such as a dose of about 10 mg daily of a compound of Formula (I) or a pharmaceutically acceptable salt thereof to the subject for a second period of time (e.g., the second week); (iii) administering a second dose, such as a dose of about 20 mg daily of a compound of Formula (I) or a pharmaceutically acceptable salt thereof to the subject for a third period of time (e.g., the third week); (iv) administering a second dose, such as a dose of about 40 mg daily of a compound of Formula (I) or a pharmaceutically acceptable salt thereof to the subject for a fourth period of time (e.g., the fourth week); (v) administering a second dose, such as a dose of about 60 mg daily of a compound of Formula (I) or a pharmaceutically acceptable salt thereof to the subject for a fifth period of time (e.g., the fifth week); (vi) administering a second dose, such as a dose of about 80 mg daily of a compound of Formula (I) or a pharmaceutically acceptable salt thereof to the subject for a sixth period of time (e.g., the sixth week); (vii) administering a second dose, such as a dose of about 100 mg daily of a compound of Formula (I) or a pharmaceutically acceptable salt thereof to the subject for a seventh period of time (e.g., the seventh week); and/or (ix) administering a second dose, such as a dose of about 120 mg daily of a compound of Formula (I) or a pharmaceutically acceptable salt thereof to the subject for a, eighth period of time (e.g., the eighth week). In some embodiments, the compositions, formulations, and dosage forms described herein make it possible to use a single tablet for each dose strength instead of needing to use multiple tablets.

For example, in certain embodiments, disclosed herein is a method of treating a disease or condition relating to aberrant function or activity of a T-type calcium channel in a subject in need thereof comprising (a) administering a first dose, such as a dose of about 20 mg or about 40 mg, of a compound of Formula (I) or a pharmaceutically acceptable salt thereof to the subject for a first period of time, wherein following administration of the first dose of the compound for the first period of time the subject has a maximum plasma drug concentration (Cₘₐₓ) ranging from about 30 ng/ml to about 130 ng/ml and/or an area under the plasma concentration-time curve from time of administration to 24 hours after administration (AUC₂₄) ranging from about 490 ng*h/mL to about 2030 ng*h/mL; (b) increasing the amount of the compound in the first dose and administering one or more increased doses of the compound to the subject to arrive at a maximum titrated dose; and (c) administering the maximum titrated dose of the compound to the subject to maintain in the subject a Cₘₐₓ ranging from about 280 ng/mL to about 470 ng/mL and/or an AUC₂₄ ranging from about 3480 ng*h/mL to about 5800 ng*h/mL. Typically, each dose is administered daily, preferably once a day, however, the frequency of administration may be altered as long as the desired Cₘₐₓ and/or AUC₂₄ values are achieved, as discussed in other sections of this application.

For instance, in certain embodiments, step (b) comprises increasing the first dose to a second dose, such as for example, a dose of about 40 mg to about 80 mg, and administering the second dose of the compound to the subject for a second period of time, wherein following administration of the second dose of the compound for the second period of time, the subject has a Cₘₐₓ ranging from about 80 ng/mL to about 300 ng/mL, such as from about 80 ng/ml to about 220 ng/mL, from about 80 ng/ml to about 130 ng/mL, or about 130 ng/ml to about 300 ng/mL, and/or an AUC₂₄ ranging from about 1220 ng*h/mL to about 4070 ng*h/mL, such as an AUC₂₄ ranging from about 1220 ng*h/mL to about 3330 ng*h/mL or from about 1220 ng*h/mL to about 2030 ng*h/mL.

In certain embodiments, step (b) may further comprise increasing the second dose to a third dose, such as a dose ranging from about 60 mg to about 100 mg, and administering the third dose of the compound to the subject for a third period of time, wherein following administration of the third dose, the subject has a Cₘₐₓ ranging from about 130 ng/ml to about 380 ng/mL, such as from 130 ng/ml to about 220 ng/mL, from about 180 ng/mL to about 300 ng/mL, or from 230 ng/mL to about 380 ng/mL and/or an AUC₂₄ ranging from about 2000 ng*h/mL to about 4700 ng*h/mL, such as from about 2000 ng*h/mL to about 3330 ng*h/mL, from about 2440 ng*h/mL to about 4070 ng*h/mL, or from about 2820 ng*h/mL to about 4700 ng*h/mL.

Also disclosed herein are embodiments further comprising increasing the third dose to a fourth dose, such as a dose of about 80 mg to about 100 mg, and administering the fourth dose of the compound to the subject for a fourth period of time, wherein following administration of the fourth dose of the compound for the fourth period of time, the subject has a Cₘₐₓ ranging from about 180 ng/mL to about 380 ng/ml, such as about 180 ng/ml to about 300 ng/ml or about 230 to about 380 ng/ml and/or an AUC₂₄ ranging from about 2440 ng*h/mL to about 4700 ng*h/mL, such as from about 2440 ng*mL/mL to about 4070 ng*h/mL or about 2820 ng*mL to about 4700 ng*h/mL. In certain aspects, the methods disclosed herein may further comprise increasing the fourth dose to a fifth dose, such as a dose of about 100 mg, and administering the fifth dose of the compound to the subject for a fifth period of time, wherein following administration of the fifth dose of the compound for the fifth period of time, the subject has a Cₘₐₓ ranging from about 230 ng/mL to about 380 ng/ml and an AUC₂₄ ranging from about 2820 ng*h/mL to about 4700 ng*h/mL.

In certain embodiments of the titrated dosing schedules disclosed herein, it is possible to increase the maximum titrated dose to maintain in the subject a Cmax greater than 470 ng/mL and/or an AUC₂₄ greater than 5800 ng*h/mL, provided the subject is able to safely tolerate the higher dose. For example, in certain embodiments, the method comprises one or more additional titration steps, to achieve a maximum titrated dose that is administered to the subject to maintain a Cₘₐₓ ranging from about 450 ng/ml to about 750 ng/mL, including, for example, about 450 ng/mL to about 650 ng/ml, about 450 ng/ml to about 550 ng/mL, or about 450 ng/ml to about 500 ng/ml, and/or an AUC₂₄ ranging from about 5500 ng*h/mL to about 9500 ng*h/mL, including, for example, about 5500 ng*h/mL to about 8500 ng*h/mL, 5500 ng*h/mL to about 7500 ng*h/mL, or about 5500 ng*h/mL to about 6500 ng*h/mL.

Also disclosed herein is a method of treating a disease or condition relating to aberrant function or activity of a T-type calcium channel in a subject in need of treatment comprising (a) administering to the subject for a first period, about 5 mg to about 40 mg per day of a compound of Formula (I) or a pharmaceutically acceptable salt thereof; (b) administering to the subject for a second period, about 10 mg to about 100 mg per day of the compound of Formula (I) or a pharmaceutically acceptable salt thereof; and (c) administering to the subject for a third period, about 20 mg to about 120 mg per day of the compound of Formula (I) or a pharmaceutically acceptable salt thereof. Typically, each of the first period, second period, and third period range from about 3 to about 9 days. However, shorter or longer periods of time may be used depending on tolerance by the subject, judgment of the clinician, and the like.

Also disclosed herein is a method of treating a disease or condition relating to aberrant function or activity of a T-type calcium channel in a subject in need of treatment comprising (a) administering to the subject for a first period, about 5 mg to about 40 mg per day of a compound of Formula (I) or a pharmaceutically acceptable salt thereof; (b) administering to the subject for a second period, about 10 mg to about 100 mg per day of the compound of Formula (I) or a pharmaceutically acceptable salt thereof; (c) administering to the subject for a third period, about 20 mg to about 120 mg per day of the compound of Formula (I) or a pharmaceutically acceptable salt thereof; (d) administering to the subject for a fourth period, about 20 mg to about 120 mg per day of the compound of Formula (I) or a pharmaceutically acceptable salt thereof; (e) administering to the subject for a fifth period, about 20 mg to about 120 mg per day of the compound of Formula (I) or a pharmaceutically acceptable salt thereof; (f) administering to the subject for a sixth period, about 20 mg to about 120 mg per day of the compound of Formula (I) or a pharmaceutically acceptable salt thereof; and (g) administering to the subject for a seventh period, about 0 mg to about 120 mg per day of the compound of Formula (I) or a pharmaceutically acceptable salt thereof. Typically, each of the first period, second period, third period, fourth period, and fifth period range from about 3 to about 9 days, the sixth periods range from about 3 to about 16 days, and the seventh period extends beyond 14 days. However, shorter or longer periods of time may be used for each period depending on tolerance by the subject, judgment of the clinician, and the like

In certain embodiments, the subject does not experience adverse events at any of the dosage levels in the titrated dosage schedule. In certain embodiments, absent administering a first dosage level for a first dosing period (e.g., about 5 mg to about 40 mg), the subject would experience adverse events at the second dosage level administered during the second dosing period (e.g., about 10 mg to about 100 mg, such as at least about 60 mg to about 100 mg). In certain embodiments, absent administering the first and second dosage level during the first and second dosing period, a subject would experience adverse events at the third dosage level administered during the third dosing period (e.g., about 20 mg to about 120 mg, such as at least about 60 mg to about 120 mg).

In certain embodiments, wherein a subject would be likely to experience adverse events at the second dosage level administered during the second dosing period absent administering the first dosage level during the first dosing period, the dosage may continue to be titrated to a dosage level that is greater than the second dosage level administered during the second dosing period, such as a dosage level that is at least about 25% greater, at least about 50% greater, at least about 75% greater, at least about 100% greater, at least about 125% greater, at least about 150% greater, at least about 175% greater, at least about 200% greater, at least about 250% greater, or at least about 300% greater than the dosage level at which adverse events are likely to occur without titration.

In certain aspects of all embodiments disclosed herein, the time period of administration, such as the first, second, third, fourth or fifth period of time, may range from about 3 to about 9 days, such as, for example, 3, 4, 5, 6, 7, 8, or 9 days. In certain aspects of the embodiments disclosed herein, the time period of administration, such as the sixth period of time, may range from about 3 to about 16 days, such as, for example, 3, 4, 5, 6, 7, 8, 9, 10, 11, 12, 13, 14, 15, or 16 days. In other aspects of the embodiments disclosed herein, of the time periods, e.g., the first, second, third, fourth, fifth, sixth, or seventh time period of administration may extend beyond 14 days.

In certain aspects, the dosage increase relative to the prior dose does not increase more than 40 mg per day. For instance, in certain embodiments, the second dose is increased no more than 40 mg per day relative to the first dose, and in certain aspects, the third dose is increased no more than 40 mg per day relative to the second dose.

In another aspect, the present disclosure provides a method of treating a disease or condition relating to aberrant function or activity of a T-type calcium channel in a subject in need thereof, the method comprising administering (e.g., once, twice, three times) daily to the subject up to about 120 mg (e.g., from about 5 mg to about 120 mg, from about 10 mg to about 120 mg, from about 15 mg to about 120 mg, from about 20 mg to about 120 mg, from about 40 mg to about 120 mg, from about 5 mg to about 100 mg, from about 10 mg to about 100 mg, from about 15 mg to about 100 mg, from about 20 mg to about 100 mg, from about 40 mg to about 100 mg, from about 5 mg to about 80 mg, from about 10 mg to about 80 mg, from about 15 mg to about 80 mg, from about 20 mg to about 80 mg, from about 40 mg to about 80 mg, from about 5 mg to about 60 mg, from about 10 mg to about 60 mg, from about 15 mg to about 60 mg, from about 20 mg to about 60 mg, or from about 40 mg to about 60 mg) of the compound of Formula (I) or a pharmaceutically acceptable salt thereof (e.g., the compound of Formula (II)).

In another aspect, the present disclosure provides a method of treating a disease or condition relating to aberrant function or activity of T-type calcium channels in a subject in need thereof, the method comprising:
(a) administering to the subject once daily for a first period (e.g., 3, 4, 5, 6, 7, 8, or 9 days), 5 mg of the compound of Formula (I) or a pharmaceutically acceptable salt thereof (e.g., the compound of Formula (II));
(b) administering to the subject once daily for a second period (e.g., 3, 4, 5, 6, 7, 8, or 9 days), 10 mg of the compound of Formula (I) or a pharmaceutically acceptable salt thereof (e.g., the compound of Formula (II)); and
(c) administering to the subject once daily for a third period (e.g., 3, 4, 5, 6, 7, 8, or 9 days), 20 mg of the compound of Formula (I) or a pharmaceutically acceptable salt thereof (e.g., the compound of Formula (II)).

In another aspect, the present disclosure provides a method of treating a disorder in a subject in need thereof, the method comprising:
(a) administering to the subject for a first period (e.g., 3, 5, 6, 7, 8, or 9 days), 20-40 mg per day of the compound of Formula (I) or a pharmaceutically acceptable salt thereof (e.g., the compound of Formula (II));
(b) administering to the subject for a second period (e.g., 3, 5, 6, 7, 8, or 9 days), 20-60 mg per day of the compound of Formula (I) or a pharmaceutically acceptable salt thereof (e.g., the compound of Formula (II)); and
(c) administering to the subject for a third period (e.g., 3, 4, 5, 6, 7, 8, or 9 days), 20-80 mg per day of the compound of Formula (I) or a pharmaceutically acceptable salt thereof (e.g., the compound of Formula (II)).

In some embodiments, the method further comprises (d) administering to the subject for a fourth period (e.g., 3, 4, 5, 6, 7, 8, or 9 days), 20-100 mg per day of the compound of Formula (I) or a pharmaceutically acceptable salt thereof (e.g., the compound of Formula (II)).

In other embodiments, the method further comprises (e) administering to the subject for a fifth period (e.g., 3, 4, 5, 6, 7, 8, or 9 days), 20-120 mg per day of the compound of Formula (I) or a pharmaceutically acceptable salt thereof (e.g., the compound of Formula (II)).

In certain embodiments, the method further comprises (f) administering to the subject for a sixth period (e.g., 3, 4, 5, 6, 7, 8, 9, 10, 11, 12, 13, 14, 15, or 16 days), 20-120 mg per day of the compound of Formula (I) or a pharmaceutically acceptable salt thereof (e.g., the compound of Formula (II)).

In certain embodiments, the method further comprises (g) administering to the subject for a seventh period (e.g., 3, 4, 5, 6, 7, 8, 9, 10, 11, 12, 13, or 14 or more days), 20-120 mg per day of the compound of Formula (I) or a pharmaceutically acceptable salt thereof (e.g., the compound of Formula (II)) as needed.

In certain aspects of these embodiments, a physician may elect not to continue to escalate the dose of the compound of Formula (I) or a pharmaceutically acceptable salt thereof, such that the dosing periods may be less than the seven periods described above. For example, there may be only 1, 2, 3, 4, 5, or 6 periods of escalating dosages needed to achieve a desired therapeutic effect. There may also be more than the seven periods of dose escalation described above as warranted by tolerance of the subject and judgment of the physician.

For instance, in certain embodiments there is disclosed a method of treating a disease or condition relating to aberrant function or activity of a T-type calcium channel in a subject in need thereof comprising (a) administering to the subject for a first period ranging from about 3 to about 9 days, about 5 mg to about 40 mg per day of a compound of Formula (I) or a pharmaceutically acceptable salt thereof, such as 20 mg or 40 mg per day; (b) administering to the subject for a second period ranging from about 3 to about 9 days, about 10 mg to about 100 mg per day of the compound of Formula (I) or a pharmaceutically acceptable salt thereof, such as 40 mg, 60 mg, or 80 mg per day; and (c) administering to the subject for a third period ranging from about 3 to about 9 days, about 20 mg to about 120 mg per day of the compound of Formula (I) or a pharmaceutically acceptable salt thereof, such as 60 mg, 80 mg, 100 mg, or 120 mg per day.

In certain aspects, disclosed herein is a method of treating a disease or condition relating to aberrant function or activity of T-type calcium channels in a subject in need thereof comprising (a) administering a first dose of about 20 mg to about 40 mg per day of a compound of Formula (I) or a pharmaceutically acceptable salt thereof to a subject once daily for a first time period; (b) increasing the amount of the compound in the first dose and administering one or more increased doses of the compound to the subject to arrive at a maximum titrated dose of about 80 mg to about 120 mg per day; and (c) administering the maximum titrated dose to the subject once daily as needed.

In related embodiments, the dosage of the compound of Formula (I) may be adjusted upward or downward in 1, 2, 3, 4, 5, 10, 15, 20 mg increments as deemed necessary by a physician depending on a subject's response to the prior dosages of compound of Formula (I).

In various embodiments of the disclosure, the methods disclosed herein comprise (a) administering a first dose of 20 mg per day for a first period of time of 3 days, (b) administering a second dose of 40 mg per day for a second period of time of 3 days, (c) administering a third dose of 60 mg per day for a third period of time of 7 days; (d) administering a fourth dose of 80 mg per day for a fourth period of time of 7 days; (e) administering a fifth dose of 100 mg per day for a fifth period of time of 7 days; and (f) thereafter administering a sixth dose of 120 mg per day as needed. In various other embodiments, the methods disclosed herein comprise (a) administering a first dose of 20 mg per day for a first period of time of 3 days, (b) administering a second dose of 40 mg per day for a second period of time of 3 days, (c) administering a third dose of 80 mg per day for a third period of time of 3 days; and (d) administering a fourth dose of 120 mg per day as needed.

In certain embodiments there is disclosed a method of treating a disease or condition relating to aberrant function or activity of T-type calcium channels in a subject in need thereof comprising (a) administering to the subject for a first period of 3 days about 20 mg per day of a compound of Formula (I) or a pharmaceutically acceptable salt thereof; (b) administering to the subject for a second period of about 3 days about 40 mg per day of the compound; (c) administering to the subject for a third period of about 3 days about 60 mg per day of the compound; (d) administering to the subject for a fourth period of about 3 days about 80 mg per day of the compound; (e) administering to the subject for a fifth period of about 3 days about 100 mg per day of the compound; and (f) administering to the subject for a sixth period of time about 120 mg per day of the compound. Instead of every 3 days, the doses can also be administered every 4, 5, or 6 days.

Also disclosed herein is a method of treating a disease or condition relating to aberrant function or activity of T-type calcium channels in a subject in need thereof comprising (a) administering to the subject for a first period of 7 days about 20 mg per day of a compound of Formula (I) or a pharmaceutically acceptable salt thereof; (b) administering to the subject for a second period of about 7 days about 40 mg per day of the compound; (c) administering to the subject for a third period of about 7 days about 60 mg per day of the compound; (d) administering to the subject for a fourth period of about 7 days about 80 mg per day of the compound; (e) administering to the subject for a fifth period of about 7 days about 100 mg per day of the compound; and (f) administering to the subject for a sixth period of time about 120 mg per day of the compound.

In certain embodiments there is disclosed a method of treating a disease or condition relating to aberrant function or activity of a T-type calcium channel in a subject in need thereof comprising (a) administering to the subject for a first period of 3 days about 40 mg per day of a compound of Formula (I) or a pharmaceutically acceptable salt thereof; (b) administering to the subject for a second period of about 3 days about 80 mg per day of the compound; and (c) administering to the subject for a third period about 120 mg per day of the compound. Instead of every 3 days, the doses can also be administered every 4, 5, or 6 days.

Also disclosed herein is a method of treating a disease or condition relating to aberrant function or activity of a T-type calcium channel in a subject in need thereof comprising (a) administering to the subject for a first period of 7 days about 40 mg per day of a compound of Formula (I) or a pharmaceutically acceptable salt thereof; (b) administering to the subject for a second period of about 7 days about 80 mg per day of the compound; and (c) administering to the subject for a third period about 120 mg per day of the compound.

In certain embodiments there is disclosed a method of treating a disease or condition relating to aberrant function or activity of a T-type calcium channel in a subject in need thereof comprising (a) administering to the subject for a first period of 7 days about 20 mg per day of a compound of Formula (I) or a pharmaceutically acceptable salt thereof; (b) administering to the subject for a second period of about 7 days about 40 mg per day of the compound; (c) administering to the subject for a third period of about 7 days about 60 mg per day of the compound; (d) administering to the subject for a fourth period of about 7 days about 80 mg per day of the compound; (e) administering to the subject for a fifth period of about 7 days about 100 mg per day of the compound; (f) administering to the subject for a sixth period of about 14 days about 120 mg per day of the compound; and (g) thereafter administering to the subject about 1-120 mg per day of the compound as needed.

In certain embodiments of the methods of treating a disease or condition relating to aberrant function or activity of a T-type calcium channel in a subject in need thereof, a physician may choose to stop escalating the dose of compound of Formula (I) or a pharmaceutically acceptable salt thereof once the subject has demonstrated the desired therapeutic effect. At this point, the physician may elect for the subject to continue the dose that the subject has been taking to achieve the desired therapeutic effect or may elect for the subject to lower the dose of the compound of Formula (I) or a pharmaceutically acceptable salt thereof in order to maintain the desired therapeutic effect. Achieving the desired therapeutic effect may be as a result of improvements in any therapeutic measure, for example, the subject's TETRAS performance score, accelerometer performance score, or Archimedes spiral task test.

In some embodiments, the subject is a human of age from birth to 100 years of age, such as from 10 to 90 years, from 20 to 70 years, from 18 to 55 years, or from 55 to 75 years of age.

In various aspects, the methods disclosed herein result in an EEG sigma frequency band reduction during NREM sleep in the subject, such as a NREM sigma band frequency reduction from a baseline of about 0.4 to 0.7, such as about 0.5 to about 0.6, or about 0.5. In certain embodiments, the methods disclosed herein result in an EEG gamma frequency band reduction during wake in an EO condition or an EC condition the subject, such as a gamma frequency band reduction as compared to a baseline gamma frequency band of at least about 25%, such as, for example, about 50% reduction.

In certain embodiments, the methods disclosed herein result in an EEG sigma frequency reduction during NREM sleep and/or an EEG gamma frequency band reduction during an EO or an EC condition in the subject when the subject is administered a dosage of the compound of Formula (I) or a pharmaceutically acceptable salt thereof resulting in a Cₘₐₓ of from about 30 ng/ml to about 470 ng/ml, such as a Cₘₐₓ ranging from about 30 to about 50 ng/mL, from about 80 to about 130 ng/ml, from about 130 to about 222 ng/mL, from about 180 to about 300 ng/ml, from about 230 to 380 ng/mL, or from about 280 to about 470 ng/mL. In certain embodiments, the methods disclosed herein result in an EEG sigma frequency reduction during NREM sleep and/or an EEG gamma frequency band reduction during an EO or an EC condition in the subject when the subject is administered a dosage of the compound of Formula (I) or a pharmaceutically acceptable salt thereof resulting in an AUC₂₄ ranging from about 490 ng*h/mL to about 5800 ng*h/mL, such as an AUC₂₄ ranging from about 490 to 820 ng*h/mL, from about 1220 to 2030 ng*h/mL, from about 2000 to 3330 ng*h/mL, from about 2440 to 4070 ng*h/mL, from about 2820 to 4700 ng*h/mL, or from about 3480 to 5800 ng*h/mL.

In certain embodiments, the methods disclosed herein result in a NREM sigma frequency reduction in the subject when the subject is administered a dosage of the compound of Formula (I) or a pharmaceutically acceptable salt thereof resulting in a Cₘₐₓ of about 5 ng/ml to about 470 ng/ml, such as a Cₘₐₓ of about 180 to about 300 ng/mL. In certain embodiments, the methods disclosed herein result in a NREM sigma frequency reduction in the subject when the subject is administered a dosage of the compound of Formula (I) or a pharmaceutically acceptable salt thereof resulting in an average plasma concentration (Cₐᵥₑ) during the EEG recording (i.e., over a period of about 24 hours) of about 10 ng/mL to about 200 ng/ml, such as a Cₐᵥₑ of about 12 to about 150 ng/mL. In certain embodiments, the methods disclosed herein result in an EO or EC gamma frequency band reduction in the subject when the subject is administered a dosage of the compound of Formula (I) or a pharmaceutically acceptable salt thereof resulting in a Cₘₐₓ of the compound of about, such as a Cₘₐₓ of about 280 to about 470 ng/mL. In certain embodiments, the methods disclosed herein result in an EO or EC gamma frequency band reduction in the subject when the subject is administered a dosage of the compound of Formula (I) or a pharmaceutically acceptable salt thereof resulting in a plasma concentration of about 75 ng/ml to about 310 ng/mL, such as a plasma concentration of about 90 to about 190 ng/mL.

In other embodiments, the disease or condition relating to aberrant function or activity of a T-type calcium channel is selected from the group consisting of psychiatric disorders (e.g., mood disorder (e.g., major depressive disorder)), pain, tremor (e.g., essential tremor), seizures (e.g., absence seizures), and epilepsy or an epilepsy syndrome (e.g., juvenile myoclonic epilepsy).

In certain aspects of all embodiments of titrated dosing schedules disclosed herein, it is possible to increase the maximum titrated dose, including, for example, increasing the maximum titrated dose above 120 mg in one or more additional titration steps, provided the subject is able to safely tolerate the higher dose.

In certain aspects of all of the embodiments of titrated dosing schedules disclosed herein, the maximum titrated dosage achieved is greater than 20 mg, greater than 40 mg, greater than 60 mg, such as about 80 mg, about 100 mg, about 120 mg, about 130 mg, about 140 mg, about 150 mg, about 160 mg, about 170 mg, about 180 mg, about 190 mg, about 200 mg, about 210 mg, or about 220 mg. In certain aspects of the invention, the maximum dosage achieved for a subject is, for example, 40 mg, 60 mg, or 80 mg if the subject has achieved a desired therapeutic outcome.

In certain aspects of all of the embodiments of titrated dosing schedules disclosed herein, the maximum titrated dosage is reached in 42 days or less, such as 31 days or less, 28 days or less, 18 days or less, 10 days or less, or 7 days or less. In certain embodiments, the maximum titrated dosage is reached in about 10 to about 42 days, such as, for example, about 36-42 days, about 22-28 days, about 16-18 days, or about 10-12 days.

### Administrations

In some embodiments, the dosage form is administered to the subject more than once a day (e.g., twice a day, three times a day, or four times a day).

In some embodiments, the dosage form is administered to the subject once a day (e.g., one 5 mg tablet once a day, one 10 mg tablet once a day, one 20 mg tablet once a day, one 40 mg tablet once a day, one 80 mg tablet once a day, or one 120 mg tablet once a day). In some embodiments, the dosage form is administered to the subject twice a day (e.g., one 5 mg tablet twice a day, one 10 mg tablet twice a day, one 20 mg tablet twice a day, one 40 mg tablet twice a day, one 80 mg tablet twice a day, or one 120 mg tablet twice a day).

In some embodiments, the dosage form is administered to the subject once a day (e.g., one 20 mg tablet once a day, two 20 mg tablets once a day, three 20 mg tablets once a day, four 20 mg tablets once a day, five 20 mg tablets once a day, or six 20 mg tablets once a day). In some embodiments, the dosage form is administered to the subject twice a day (e.g., one 10 mg tablet twice a day, one 20 mg tablet twice a day, two 20 mg tablets twice a day, three 20 mg tablets twice a day, four 20 mg tablets twice a day, five 20 mg tablets twice a day, or six 20 mg tablets twice a day).

In some embodiments, the dosage form is administered to the subject every other day. In other embodiments, the dosage form is administered to the subject once a week or twice weekly.

In some embodiments, methods of the present disclosure comprise administering to a subject in need thereof a titrated dose of the compound of formula (I) or (II). In some embodiments, the maximum titrated dose is 60 mg per day or 100 mg per day. In one embodiment, the maximum titrated dose is 60 mg per day.

In certain embodiments, about 1 mg to 10 mg, such as 2 mg to 5 mg, of the compound of Formula (I) or a pharmaceutically acceptable salt thereof (e.g., the compound of Formula (II), e.g., PRAX-944 HCl) is administered to the subject daily. In certain embodiments, about 1 mg to 20 mg, such as 5 mg to 10 mg, of the compound of Formula (I) or a pharmaceutically acceptable salt thereof (e.g., the compound of Formula (II), e.g., PRAX-944 HCl) is administered to the subject daily. In certain embodiments, about 1 mg to 30 mg, such as 10 mg to 20 mg, of the compound of Formula (I) or a pharmaceutically acceptable salt thereof (e.g., the compound of Formula (II), e.g., PRAX-944 HCl) is administered to the subject daily. In certain embodiments, about 1 mg to 40 mg, such as 20 mg to 30 mg, of the compound of Formula (I) or a pharmaceutically acceptable salt thereof (e.g., the compound of Formula (II), e.g., PRAX-944 HCl) is administered to the subject daily. In certain embodiments, about 1 mg to 50 mg, such as 30 mg to 40 mg, of the compound of Formula (I) or a pharmaceutically acceptable salt thereof (e.g., the compound of Formula (II), e.g., PRAX-944 HCl) is administered to the subject daily. In certain embodiments, about 1 mg to 60 mg, such as 40 mg to 50 mg, of the compound of Formula (I) or a pharmaceutically acceptable salt thereof (e.g., the compound of Formula (II), e.g., PRAX-944 HCl) is administered to the subject daily. In certain embodiments, about 1 mg to 70 mg, such as 50 mg to 60 mg, of the compound of Formula (I) or a pharmaceutically acceptable salt thereof (e.g., the compound of Formula (II), e.g., PRAX-944 HCl) is administered to the subject daily. In certain embodiments, about 1 mg to 80 mg, such as 60 mg to 70 mg, of the compound of Formula (I) or a pharmaceutically acceptable salt thereof (e.g., the compound of Formula (II), e.g., PRAX-944 HCl) is administered to the subject daily. In certain embodiments, about 1 mg to 90 mg, such as 70 mg to 80 mg, of the compound of Formula (I) or a pharmaceutically acceptable salt thereof (e.g., the compound of Formula (II), e.g., PRAX-944 HCl) is administered to the subject daily. In certain embodiments, about 1 mg to 100 mg, such as 80 mg to 90 mg, of the compound of Formula (I) or a pharmaceutically acceptable salt thereof (e.g., the compound of Formula (II), e.g., PRAX-944 HCl) is administered to the subject daily. In certain embodiments, about 1 mg to 110 mg, such as 90 mg to 100 mg, of the compound of Formula (I) or a pharmaceutically acceptable salt thereof (e.g., the compound of Formula (II), e.g., PRAX-944 HCl) is administered to the subject daily. In certain embodiments, about 1 mg to 120 mg, such as 100 mg to 110 mg, of the compound of Formula (I) or a pharmaceutically acceptable salt thereof (e.g., the compound of Formula (II), e.g., PRAX-944 HCl) is administered to the subject daily.

In some embodiments, the dosage form, upon administration to the subject, has substantially the same bioavailability, e.g., with respect to the rate (maximum plasma drug concentration; Cmax) and extent (area under the plasma concentration-time curve; AUC) of absorption of PRAX-944 as compared to multiple small, round 20-mg tablets.

In some embodiments, the dosage form, upon administration to the subject, has a reduced Cₘₐₓ value than a reference oral dosage form (e.g., a dosage form with any intended release rate profile e.g., modified release rate profile, a dosage form that does not have a modified release rate profile, a dosage form that does not have a modified-release polymer, e.g., an HPMC polymer). In some embodiments, the dosage form, upon administration to the subject, has a greater tₘₐₓ value than a reference oral dosage form (e.g., a dosage form with any intended release rate profile e.g., modified release rate profile, a dosage form that does not have a modified release rate profile, a dosage form that does not have a modified-release polymer, e.g., an HPMC polymer).

### Tremor

The methods described herein can be used to treat tremor, for example a dosage or composition disclosed herein can be used to treat cerebellar tremor or intention tremor, dystonic tremor, essential tremor, orthostatic tremor, parkinsonian tremor, physiological tremor, or rubral tremor. Tremor includes hereditary, degenerative, and idiopathic disorders such as Wilson's disease (hereditary), Parkinson's disease (degenerative), and essential tremor (idiopathic); metabolic diseases; peripheral neuropathies (associated with Charcot-Marie-Tooth, Roussy-Levy, diabetes mellitus, complex regional pain syndrome); toxins (nicotine, mercury, lead, carbon monoxide, manganese, arsenic, toluene); drug-induced (neuroleptics tricyclics, lithium, cocaine, alcohol, adrenaline, bronchodilators, theophylline, caffeine, steroids, valproate, amiodarone, thyroid hormones, vincristine); and psychogenic disorders. Clinical tremor can be a neuropathic tremor, and can be classified into physiologic tremor, enhanced physiologic tremor, essential tremor syndromes (including classical essential tremor), primary orthostatic tremor, task- and position-specific tremor, dystonic tremor, parkinsonian tremor, cerebellar tremor, Holmes' tremor (i.e., rubral tremor), palatal tremor, toxic or drug-induced tremor, and psychogenic tremor. The tremor may be familial tremor.

Tremor is an involuntary, rhythmic, oscillation of one or more body parts (e.g., hands, arms, eyes, face, head, vocal folds, trunk, and/or legs).

Cerebellar tremor or intention tremor is a slow, broad tremor of the extremities that occurs after a purposeful movement. Cerebellar tremor is caused by lesions in or damage to the cerebellum or pathways resulting from, e.g., tumor, stroke or other focal lesion disease (e.g., multiple sclerosis) or a neurodegenerative disease.

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest or certain sensory maneuvers.

Essential tremor or benign essential tremor is the most common type of tremor. Essential tremor may be mild and nonprogressive in some, and may be slowly progressive, starting on one side of the body but typically affecting both sides. The hands are most often affected, but the head, voice, tongue, legs, and trunk may also be involved. Tremor frequency may decrease as the person ages, but severity may also increase. Heightened emotion, stress, fever, physical exhaustion, or low blood sugar may trigger tremors and/or increase their severity. Symptoms generally evolve over time and can be both visible and persistent following onset. Tremors, including essential tremor, can interfere with any or all of a person's activities of daily living, such as for example personal hygiene, cooking, eating, dressing, making home repairs, and interacting with other people. Tremors, including essential tremor, can interfere with career choice or job performance (e.g., typing on computer or mobile telephone, using tools, sewing, restaurant work (cooking or serving), caring for others (medical or veterinary work), or any work that requires movement may be difficult). Tremors can also have profound emotional effects, such as fear of tremor being discovered, fear of others' reactions, or fear of rejection.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is typically seen as a “pill-rolling” action of the hands that may also affect the chin, lips, legs, and trunk. Onset of parkinsonian tremor typically begins after age 60. Movement dysfunction typically starts in one limb or on one side of the body and can progress to include the other side.

Rubral tremor is characterized by coarse slow tremor which can be present at rest, at posture, and with intention. The tremor is associated with conditions that affect the red nucleus in the midbrain, such as a stroke.

In some embodiments, the tremor is selected from essential tremor, Parkinson's tremor, or Cerebellar tremor. In some embodiments, the tremor is essential tremor.

### Essential Tremor

The compositions described herein are useful in the treatment of essential tremor (ET). ET is the most common adult movement disorder, affecting up to 2% of the United States population (approximately 7 million Americans) (Louis and Ottman 2014). ET is characterized by a 5 to 12 Hz postural and kinetic tremor (i.e., tremor during voluntary movement) in the upper limbs (Bhidayasiri 2005, Louis 2009). The most characteristic clinical feature is kinetic tremor of the arms and hands, but tremor may also occur in the head and voice, and less commonly face, legs, and trunk. The diagnosis of ET is based on medical history and neurological exam as described in The International Parkinson and Movement Disorders Society Consensus Statement on the Classification of Tremors (Bhatia et al 2018).

There is a range of severity of ET; some patients require no treatment, whereas others have severe disability with impairment in activities of daily living such as dressing and eating. ET, by definition, is generally not associated with other neurological signs (Deuschl et al 2011), although there is increasing recognition that ET can also be associated with additional motor features, such as postural instability, dystonia, mild to moderate gait ataxia, and eye motion abnormalities (Louis 2009). ET also is associated with an elevated prevalence of comorbid psychiatric disorders, including anxiety and depression (Dogu et al 2005, Miller et al 2007). ET often worsens over time, with more severe tremor over the course of years to decades and corresponding worsening disability (Louis 2019).

ET can be sporadic, but a family history of an autosomal-dominant pattern of inheritance is commonly encountered (Clark and Louis 2018) and importantly, variants in the calcium voltage-gated channel subunit alpha1 G (CACNA1G) gene, which encodes the T-type Ca2+ channel isoform Cav3.1 have been identified as the cause of ET in at least 3 families (Odgerel et al 2019). The importance of the CACNA1G gene to cerebellar development and function is further highlighted by the observation that variants in this gene can also cause childhood cerebellar atrophy (Chemin et al 2018) and spinocerebellar ataxia type 42 (Coutelier et al 2015). The functional consequences of these genetic variants are consistent with the expression of T-type Ca2+ channels in the cerebellum and the distal CTC circuit and their physiological contribution to the oscillatory burst firing in the thalamus that is synchronized with and, the Sponsor hypothesizes, drives the clinically observable tremor (Milosevic et al 2018).

Propranolol is the only Food and Drug Administration (FDA)-approved orally administered treatment indicated for the treatment of ET. Propranolol was originally developed for hypertension, and in the treatment of ET exhibits limited efficacy with side effects (e.g., bradycardia) that often lead to discontinuation (Hedera 2017, Louis 2015). The unmet medical need in ET has resulted in the off-label use of medications in multiple drug classes, including anticonvulsants, barbiturates, benzodiazepines, antipsychotics, and others, with limited appreciable therapeutic benefit for patients with ET. A recent evidenced-based review concluded that only propranolol, primidone, and topiramate had sufficient evidence to support efficacy amongst 28 drugs studied for ET (Ferreira et al 2019). Surgical interventions that interrupt CTC burst activity, such as deep brain stimulation or focused ultrasound, are effective treatments for ET but carry risks, such as sensory disturbances, hemiparesis, dysarthria, ataxia, gait disturbances, delirium, cognitive decline, tissue damage, venous thromboembolic events, and intracerebral hemorrhage (Dallapiazza et al 2019, Insightec, Inc. 2016).

In some embodiments, the present disclosure also provides a method of treating essential tremor that comprises administering to a subject in need thereof a composition described herein. In some embodiments, the method results in reduction of the essential tremor as assessed by The Essential Tremor Rating Assessment Scale (TETRAS) score. The term “The Essential Tremor Rating Assessment Scale (TETRAS)”, as used herein, refers to a scale developed to quantify severity of essential tremor and its impact on daily activities. It has an activities of daily living (ADL) section and a performance section. The ADL section has 12 items rated between 0 to 4, and the performance section has 9 items rated between 0 to 4.

In some embodiments, the reduction of the essential tremor is assessed by The Essential Tremor Rating Assessment Scale (TETRAS) upper limb score.

In some embodiments, the reduction of the essential tremor is assessed by TETRAS performance subscale score or TETRAS performance individual items.

In some embodiments, the subjects treated in accordance with the methods provided by the present disclosure have a moderate essential tremor (i.e., TETRAS score of 10-15). In some embodiments, the subjects treated herein have a TETRAS score of about 10 to about 15, or a TETRAS score of about 12, prior to treatment in accordance with the methods of the present disclosure. Treatment using a composition described herein may reduce the TETRAS score of the subject. In some embodiments, subjects treated with a composition described herein experience a mean reduction in TETRAS score of about 2 to 5, e.g., about 3. In some embodiments, subjects treated with a composition described herein experience a mean reduction in TETRAS score of about 30% to about 50%, e.g., about 40%.

In some embodiments, the present disclosure also provides a method of treating essential tremor that comprises administering to a subject in need thereof a composition described herein. In some embodiments, the method results in reduction of the essential tremor as assessed by accelerometer-based score, e.g., accelerometer-based upper limb score. In some embodiments, the method results in reduction of the essential tremor as assessed by CGI score.

In some embodiments, the essential tremor is upper limb tremor.

In some embodiments, the subjects are selected for treatment with the compound of formula (I) due to a clinical diagnosis of essential tremor. In some embodiments, the subjects selected for treatment with the compound of formula (I) have essential tremor, but do not have intention tremor.

The efficacy of the compound or composition described herein for treating essential tremor can be measured by methods known in the art, such as the methods described in the following references: Ferreira, J. J. et al., “MDS Evidence-Based Review of Treatments for Essential Tremor,” Mov. Disord. 2019 July; 34 (7): 950-958; Elble, R. et al., “Task Force Report: Scales for Screening and Evaluating Tremor,” Mov. Disord. 2013 November; 28 (13): 1793-800; Deuschl G. et al., “Treatment of patients with essential tremor,” Lancet Neurol. 2011; 10:148-61; and Reich S. G. et al., “Essential Tremor,” Med. Clin. N. Am. 2019; 103:351-356. The disclosures of the references are herein incorporated in their entirety.

In some embodiments, the methods described herein result in at least 25% reduction in the upper limb tremor score, wherein the tremor score may be converted to amplitude, as compared to a baseline. For example, in certain embodiments, the methods described herein result in about 40% mean reduction in tremor amplitude as measured by The Essential Tremor Rating Assessment Scale (TETRAS) upper limb score, described, for example, in Elble, R. J., “The Essential Tremor Rating Assessment Scale,” J. Neurol. Neuromed. 2016; 1 (4): 34-38. In some embodiments, the methods described herein result in at least 25% reduction in TETRAS performance score as compared to the baseline. In some embodiments, the methods described herein result in at least 35% average reduction in symptom severity as compared to the baseline, as measured by TETRAS performance score.

### Parkinson's Disease

The compositions described herein are useful in the treatment of Parkinson's Disease (PD). PD is a debilitating neurodegenerative disorder with about 1 million diagnosed patients in the United States (US) and about 10 million worldwide. Approximately 60,000 patients are diagnosed with PD each year in the US alone (Marras et al 2018). The diagnosis of PD is most often made based upon medical history and neurological exam as described in the International Parkinson and Movement Disorders Society (MDS) Clinical Diagnostic Criteria for Parkinson's Disease (Postuma et al 2015).

PD is characterized by slow movement (bradykinesia), in combination with rest tremor (4-6 Hz) and/or rigidity (Postuma et al 2015). Symptoms are typically unilateral in onset, mild and nondisabling, but progress over time in severity and to involving both sides of the body, often impairing activities of daily living (ADLs), such as dressing and eating. In addition to the cardinal symptoms above, patients with PD can develop abnormal posturing (dystonia), severe forward flexion of the thoracolumbar spine (camptocormia), slurred speech (dysarthria), gait instability, and freezing of gait. Non-motor symptoms, such as impaired smell, dream re-enactment behavior (rapid eye movement [REM] sleep behavior disorder [RBD], constipation, depression, psychosis, and dementia also play a pivotal role in the daily life of PD patients (Jankovic and Tan 2020).

As degeneration of dopaminergic neurons in the substantia nigra pars compacta (SNc) of PD patients results in loss of dopaminergic output, the cornerstone of treatment for PD is dopaminergic replacement therapy (Armstrong and Okun 2020). L-dopa or levodopa, a formulation of replacement dopamine, is considered the gold standard treatment of PD. Levodopa is highly effective in improving motor symptoms of bradykinesia and rigidity, particularly early in the disease, but has a variable impact on tremor. Most other common PD medications mainly reduce dopamine metabolism or are dopamine receptor agonists.

While effective, side effects of chronic dopaminergic therapy include motor complications (dyskinesias or involuntary choreiform movements), impulse control disorders, and psychosis (Poewe et al 2017). These adverse effects themselves can be disabling, often necessitating medications to counteract the adverse effects. Thus, symptom management using non-dopaminergic therapeutics that have a better safety and tolerability profile and are needed.

Adjunctive oral non-dopaminergic therapies that alleviate PD symptoms could also reduce the need for dose escalations of dopaminergic therapies.

Surgical therapy, namely deep brain stimulation (DBS), can be very successful for uncontrolled tremors and motor complications and typically impacts basal ganglia function downstream of the nigrostriatal pathway (Fasano et al 2012). Depending on the patient's specific symptoms, DBS electrodes can be placed in the globus pallidus interna, nucleus ventralis intermedius of the thalamus, or the subthalamic nucleus (STN), and may affect aberrant burst firing in these areas (see below). DBS is typically reserved for patients who respond to but who have maximized dopaminergic therapy and continue to have troubling motor symptoms. Further, DBS carries significant risks associated with brain surgery, including intracerebral hemorrhage, infection, hemiparesis, and cognitive decline (Pahwa et al 2006). The prospect of neurosurgery in later years is often anxiety-provoking or contraindicated (e.g., due to the presence of cognitive impairment), and many patients are not treated with DBS. Thus, there is a clinical need for a pharmacological therapy that could mimic the effects of DBS. By modulating T-type Ca2+ channels, PRAX-944 may modulate burst firing downstream of the SNc degeneration.

Animal models and human PD patient data demonstrate that degeneration of nigral dopaminergic neurons converts the downstream STN from a tonic firing to an aberrant bursting phenotype (Ammari et al 2011, Tai et al 2011, Pan et al 2016). This bursting activity drives symptoms of bradykinesia and rigidity in animal models, and blockade of T-type Ca²⁺ channels has been shown to reduce this phenotype (Tai et al 2011, Yang et al 2014, Pan et al 2016). T-type Ca²⁺ channels are expressed widely throughout the brain, including in the STN (Talley et al 1999, Weiss and Zamponi 2019). Eliminating this burst firing through blockade of T-type Ca²⁺ channels has been shown to improve motor function in a 6-hydroxydopamine rat model of PD (Pan et al 2016). Thus, an inhibitor of T-type Ca2+ channels could offer a non-surgical and non-dopaminergic treatment option to alleviate motor symptoms in PD.

### Epilepsy and Epilepsy Syndromes

The compositions described herein are useful in the treatment of epilepsy and epilepsy syndromes. Epilepsy is a central nervous system disorder in which nerve cell activity in the brain becomes disrupted, causing recurrent seizures which can manifest as abnormal movements, periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure, involving multiple areas of the brain, or a partial or focal seizure. All areas of the brain are involved in a generalized seizure. A person experiencing a generalized seizure may cry out or make some sound, stiffen for several seconds to a minute and then have rhythmic movements of the arms and legs. The eyes may be open, and/or the person may appear not to be breathing and turn blue. The return to consciousness may be gradual, and the person may be confused from minutes to hours. The following are the main types of generalized seizures: tonic-clonic, tonic, clonic, myoclonic, myoclonic-tonic-clonic, myoclonic-atonic, atonic, and absence (typical, atypical, myoclonic, eyelid myoclonia) seizures, and epileptic spasms. In a partial or focal seizure, only part of the brain is involved, so only part of the body is affected. Depending on the part of the brain having abnormal electrical activity, symptoms may vary.

Epilepsy, as described herein, includes a generalized, partial, complex partial (e.g., seizures involving only part of the brain, but where consciousness is compromised), tonic clonic, clonic, tonic, refractory seizures, status epilepticus, absence seizures, febrile seizures, or temporal lobe epilepsy.

The compositions described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises epileptic encephalopathy, Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency. In some embodiments, the epilepsy syndrome is childhood absence epilepsy (CAE). In some embodiments, the epilepsy syndrome is juvenile absence epilepsy (JAE). In some embodiments, the epilepsy syndrome is Lennox-Gastaut syndrome. In some embodiments, the epilepsy syndrome is SLC6A1 epileptic encephalopathy. In some embodiments, the epilepsy syndrome is associated with mutations in the genes that code for T-type calcium channels (e.g., CACNAIG, EEF1A2, and GABRG2 for genetic generalized epilepsy (GGE) and LGI1, TRIM3, and GABRG2 for non-acquired focal epilepsy (NAFE)), as discussed, for example, in Feng, Y C A, et al., “Ultra-Rare Genetic Variation in the Epilepsies: A Whole-Exome Sequencing Study of 17,606 Individuals,” Am. J. Human Gen. 2019; 105 (2): 267-282. In some embodiments, the epilepsy syndrome is Doose syndrome or myoclonic astatic epilepsy. In some embodiments, the epilepsy syndrome is epileptic encephalopathy with continuous spike and wave during sleep (CSWS). In some embodiments, the epilepsy syndrome is Landau Kleffner Syndrome (LKS). In some embodiments, the epilepsy syndrome is Jeavons syndrome.

### Absence Seizures

Absence seizures are one of the most common seizure types in patients with idiopathic generalized epilepsy (IGE) (Berg et al., Epilepsia 2000). Absence seizures are relatively brief, non-convulsive seizures characterized by abrupt onset of loss of awareness and responsiveness, usually lasting between 10-30 seconds in duration, with a rapid return to normal consciousness without post-ictal confusion. The seizures are characterized on an accompanying EEG recording by the abrupt onset and offset of generalized 1-6 Hz (e.g., 3 Hz) spike and wave discharges. Absence seizure often occur multiple times per day, interrupt learning and psychosocial functioning, and present a risk of injury because of the frequent episodes of loss of awareness. Typically, absence seizures begin in early childhood and remit by teenage years. However, in a minority of patients they persist into adulthood where they are often drug resistant and may be accompanied by other seizure types such as generalized tonic-clonic seizures. In these adult patients, the absence seizures are usually highly disabling, in particular by disqualifying the sufferer from obtaining a motor vehicle license or pursuing occupations and hobbies in which the seizures-associated periods of loss of awareness pose a safety risk and are associated with significant psychosocial disabilities (Wirrell et al., 1997).

While there is a common perception that absence seizures are relatively “easy” to treat, a randomized control trial in patients with childhood absence epilepsy showed that even the most effective anti-epileptic drugs, ethosuximide and valproate, only completely controlled the seizures in 53% and 58% of patients respectively at 16 weeks as assessed by video-EEG recordings (Glauser et al., 2010), and 45% and 44% respectively at 12 months (Glauser et al., 2013). Lamotrigine, the other AED commonly used to treat absence seizures, only controlled the seizures in 29% of patients at 16 weeks, and 21% of patients at 12 months. Furthermore, both ethosuximide and valproate are commonly associated with intolerable side effects (occurring in 24% of patients treated with either of these drugs) (Glauser et al., 2010), and the latter is now generally considered to be contraindicated in girls and women of childbearing potential. Other treatment options for absence seizures are limited, with only benzodiazepines having established efficacy, and these are commonly poorly tolerated due to sedative and cognitive side effects. Absence seizures persisting into adult life are particularly difficult to treat, with patients often being treated with multiple drugs resulting in significant side-effects, sometimes without attaining seizure control.

There is abundant evidence that low threshold (T-type) calcium channels play a role in the generation and maintenance of absence seizures, being a key component of the oscillatory burst firing that occurs in thalamocortical neurons during absence seizures (Pinault and O'Brien, 1997). In some embodiments, the present disclosure is directed towards a method for treating absence seizures with a composition described herein. In some embodiments, the absence seizures are refractory absence seizures. In some embodiments, the absence seizures are refractory to an anti-epileptic drug (e.g., ethosuximide, valproic acid, or lamotrigine).

In some embodiments, the subject has epilepsy. In some embodiments, the absence seizures are atypical absence seizures. In some embodiments, the absence seizures comprise adult absence seizures, juvenile absence seizures, or childhood absence seizures.

In some embodiments, the methods described herein further comprise identifying a subject having absence seizures.

In some embodiments, the present disclosure provides a method of treating a generalized epileptic syndrome with absence seizures in a subject in need thereof, the method comprising administering to the subject an effective amount of a composition described herein. In some embodiments, the method results in reduction of the number of seizures.

In some embodiments, the present disclosure provides a method of treating a generalized epileptic syndrome with absence seizures in a subject in need thereof, the method comprising administering to the subject an effective amount of a composition described herein.

In some embodiments, the method of treating a generalized epileptic syndrome with absence seizures provided by the present disclosure results in reduction of the mean or total seizure duration.

In some embodiments, the method of treating a generalized epileptic syndrome with absence seizures provided by the present disclosure results in reduction of the seizure frequency, duration or both as measured by Electroencephalogram (EEG).

In some embodiments, the method of treating a generalized epileptic syndrome with absence seizures provided by the present disclosure results in a reduction of the mean seizure duration as measured by EEG.

In some embodiments, the method of treating a generalized epileptic syndrome with absence seizures provided by the present disclosure results in a reduction of the cumulative seizure duration as measured by EEG.

In some embodiments, the method of treating a generalized epileptic syndrome with absence seizures provided by the present disclosure results in a reduction of the total time with 2.5-4 Hz spike wave discharges after hyperventilation and photic stimulation challenges as measured by EEG.

In some embodiments, the method of treating a generalized epileptic syndrome with absence seizures provided by the present disclosure results in a reduction of global severity as measured by Clinical Global Impression-Severity (CGI-S) or Clinical Global Impression-Improvement (CGI-I) scores. CGI-S is a 7-point scale test to rate the severity of the patient's illness at the time of assessment, relative to the clinician's past experience with patients with the same diagnosis. CGI-I is a 7-point scale test to evaluate the improvement of the patient's illness relative to the baseline.

In some embodiments, the method of treating a generalized epileptic syndrome with absence seizures provided by the present disclosure results in a reduction of the number of seizures.

In some embodiments, the method of treating a generalized epileptic syndrome with absence seizures provided by the present disclosure results in a reduction of the seizure density as measured by Electroencephalogram (EEG).

In some embodiments, the method of treating a generalized epileptic syndrome with absence seizures provided by the present disclosure results in a reduction of the mean seizure duration as measured by EEG.

### Genetic Epilepsies

In some embodiments, the epilepsy or epilepsy syndrome is a genetic epilepsy or a genetic epilepsy syndrome. In some embodiments, the epilepsy or epilepsy syndrome is genetic generalized epilepsy. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) prior to administration of a composition described herein.

In one aspect, the present invention features a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) comprising administering to a subject in need thereof a composition described herein.

A composition of the present invention may also be used to treat an epileptic encephalopathy, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, WWOX, CACNAIG, CACNA1H, and CACNA1I prior to administration of a composition described herein.

A composition of the present invention may also be used to treat an epileptic encephalopathy, wherein the subject has a mutation in one or more of ADSL, ALDH5A1, ALDH7A1, ALG13, ARGI, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBL1XR1, AMT, GCSH, GLDC, FLNA, PTEN, and RANBP2.

In some embodiments, the methods described herein further comprise identifying a subject having a mutation in one or more of ADSL, ALDH5A1, ALDH7A1, ALG13, ARGI, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBL1XR1, AMT, GCSH, GLDC, FLNA, PTEN, and RANBP2.

A composition of the present invention may also be used to treat an epileptic encephalopathy, wherein the subject has a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBL1XR1, TCF4, TPP1 (CLN2), TSC1, TSC2, UBE3A, WDR45, WWOX, and ZEB2.

In some embodiments, the methods described herein further comprise identifying a subject having a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBL1XR1, TCF4, TPP1 (CLN2), TSC1, TSC2, UBE3A, WDR45, WWOX, and ZEB2.

A composition of the present invention may also be used to treat an epileptic encephalopathy, wherein the subject has a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBL1XR1, TCF4, TPP1, TSC1, TSC2, UBE3A, WDR45, and ZEB2.

In some embodiments, the methods described herein further comprise identifying a subject having a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1HI, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBL1XR1, TCF4, TPP1, TSC1, TSC2, UBE3A, WDR45, and ZEB2.

### Mood Disorders

Also provided herein are methods for treating a psychiatric disorder such as a mood disorder, for example clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD) and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly considered decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent feeling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, and suicidal behavior refer to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, and/or incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, and saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case-to-case basis. Symptoms of depression, and relief of the same, may be ascertained by a physician or psychologist (e.g., by a mental state examination).

In some embodiments, the mood disorder is selected from depression, major depressive disorder, bipolar disorder, dysthymic disorder, anxiety disorders, stress, post-traumatic stress disorder, bipolar disorder, and compulsive disorders. In some embodiments, the mood disorder is major depressive disorder.

In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. Therapeutic effect can be assessed across a specified treatment period. For example, therapeutic effect can be determined by a decrease from baseline in HAM-D total score after administering a composition described herein (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a composition described herein), is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a composition described herein is greater than or equal to 23. In some embodiments, the baseline score is at least 10, 15, or 20. In some embodiments, the HAM-D total score of the subject after treatment with a composition described herein is about 0 to 10 (e.g., less than 10; 0 to 10, 0 to 6, 0 to 4, 0 to 3, 0 to 2, or 1.8). In some embodiments, the HAM-D total score after treatment with a composition described herein is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a composition described herein. In some embodiments, the decrease in the baseline HAM-D total score to HAM-D total score after treatment with a composition described herein is at least 1, 2, 3, 4, 5, 7, 10, 25, 40, or 50). In some embodiments, the percentage decrease in the baseline HAM-D total score to HAM-D total score after treatment with a composition described herein is at least 50% (e.g., 60%, 70%, 80%, or 90%). In some embodiments, therapeutic effect is measured as a decrease in the HAM-D total score after treatment with a composition described herein relative to the baseline HAM-D total score.

In some embodiments, the method of treating a depressive disorder, e.g., major depressive disorder, provides a therapeutic effect (e.g., as measured by reduction in the HAM-D score within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with a composition described herein. In some embodiments, therapeutic effect is a decrease from baseline in HAM-D total score after treatment with a composition described herein. In some embodiments, the HAM-D total score of the subject before treatment with a composition described herein is at least 24. In some embodiments, the HAM-D total score of the subject before treatment with a composition described herein is at least 18. In some embodiments, the HAM-D total score of the subject before treatment with a composition described herein is between and including 14 and 18. In some embodiments, the decrease in HAM-D total score after treating the subject with a composition described herein relative to the baseline HAM-D total score is at least 10. In some embodiments, the decrease in HAM-D total score after treating the subject with a composition described herein relative to the baseline HAM-D total score is at least 15. In some embodiments, the HAM-D total score associated with treating the subject with a composition described herein is no more than a number ranging from 6 to 8. In some embodiments, the HAM-D total score associated with treating the subject with a composition described herein is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, therapeutic effect is a decrease from baseline in CGI score at the end of a treatment period (e.g., 14 days after administration).

A therapeutic effect for major depressive disorder can be determined by a reduction in Montgomery-Asberg Depression Rating Scale (MADRS) score exhibited by the subject. For example, the MADRS score can be reduced within 4, 3, 2, or 1 days; or 96, 84, 72, 60, 48, 24, 20, 16, 12, 10, 8 hours or less. The MADRS is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders. In some embodiments, therapeutic effect is a decrease from baseline in MADRS score at the end of a treatment period (e.g., 14 days after administration).

### Pain

The dosage forms and compositions described herein may be useful in the treatment of pain. In some embodiments, the pain comprises acute pain, chronic pain, neuropathic pain, inflammatory pain, nociceptive pain, central pain (e.g., thalamic pain), or migraine. In some embodiments, the pain comprises acute pain or chronic pain. In some embodiments, the pain comprises neuropathic pain, inflammatory pain, or nociceptive pain. In some embodiments, the pain comprises central pain (e.g., thalamic pain). In some embodiments, the pain comprises migraine.

In some embodiments, the methods described herein further comprise identifying a subject having pain (e.g., acute pain, chronic pain, neuropathic pain, inflammatory pain, nociceptive pain, central pain (e.g., thalamic pain), or migraine) prior to administration of a dosage form or composition described herein (e.g., a dosage form or composition including the compound of Formula (I) or a pharmaceutically acceptable salt thereof (e.g., the compound of Formula (II), e.g., PRAX-944 HCl)).

### Ataxia

The dosage forms and compositions described herein may be useful in the treatment of ataxia. Ataxia, including both cerebellar ataxia and spinal ataxia (e.g., posterior spinal ataxia), generally involves the loss or failure of coordination. Patients exhibiting ataxia may have difficulty regulating the force, range, direction, velocity, and rhythm involved in posture, balance, and limb movement. Ataxia of the trunk, for example, can result in increased postural sway, and an inability to maintain the center of gravity over the base of support. Ataxia and primary or secondary symptoms of ataxic gait and tremor of the limbs may be accompanied by speech disturbance, dysphagia, abnormal ventilation and speech, and involuntary eye movements, dystonia, pyramidal or extrapyramidal symptoms, thereby substantially interfering with the activities of daily life.

As noted above, ataxia may result from a wide range of underlying diseases and conditions in a patient, including cerebellar and neurodegenerative disorders and diseases resulting from chronic or long-term exposure to toxins. Symptoms of ataxia may result from a wide range of diseases, disorders, and environmental factors, including infectious diseases, metabolic diseases, neurodegenerative diseases, genetic diseases, vascular diseases, neoplastic diseases, demyelinating diseases, neuromuscular diseases, and diseases resulting from long-term or chronic exposure to toxins (including drugs and alcohol), among a variety of others; in one embodiment, for example, the ataxia is the result of a metabolic disease, a neurodegenerative disease, a vascular disease, a neuromuscular disease, or a disease resulting from long-term or chronic exposure to toxins. Diseases, disorders, syndromes, and conditions that may result in ataxic symptoms that may be treated according to the methods described herein include, but are not limited to, amyotrophic lateral sclerosis, benign paroxysmal positional vertigo, cerebellar ataxia type 1 (autosomal recessive), cerebellar ataxias (autosomal recessive), cerebellar ataxias (dominant pure), cerebellar cortical atrophy, cerebellar degeneration (subacute), cerebellar dysfunction, cerebellar hypoplasia, cerebellar hypoplasia (endosteal sclerosis), cerebellar hypoplasia (tapetoretinal degeneration), cerebelloparenchymal autosomal recessive disorder 3, cerebelloparenchymal disorder V, cerebellum agenesis (hydrocephaly), cerebral amyloid angiopathy (familial), cerebral palsy, demyelinating disorder, dorsal column conditions, dysautonomia, dysequilibrium syndrome, dysethesis, endocrine diseases, diseases caused by chronic exposure to toxins (e.g., alcohol, drugs, antiepileptics, neuroleptics), Fragile X/Tremor ataxia syndrome, Friedreich's ataxia, frontal lobe dysfunction, genetic diseases, granulomatous angiitis of the central nervous system, Hallervorden-Spatz disease, hereditary motor and sensory neuropathy, hydrocephalus (e.g., low or normal pressure), hypotonia, congenital nystagmus, ataxia and abnormal auditory brainstem response, infantile onset spinocerebellar ataxia, Machado-Joseph disease, Meniere's disease, metabolic disorders, Miller Fisher Syndrome, Minamata disease, multiple sclerosis, muscular dystrophy, Myoclonus-ataxia, neurodegenerative diseases, olivopontocerebellar atrophy, paraneoplastic disorders, parkinsonism (atypical), peroneal muscular atrophy, phenyloin toxicity, posterior column ataxia with retinitis pigmentosa, post-polio syndrome, severe damage to the brain (caused by, e.g., head injury, brain surgery, multiple sclerosis or cerebral palsy, chronic alcohol/drug abuse, chronic exposure to toxins, viral infections, or brain tumor), spastic hemiparesis, spastic paraplegia 23, spastic paraplegia glaucoma precocious puberty, SPG, spinocerebellar ataxia, spinocerebellar ataxia (amyotrophy-deafness), spinocerebellar ataxia (dysmorphism), spinocerebellar ataxia 11, spinocerebellar ataxia 17, spinocerebellar ataxia 20, spinocerebellar ataxia 25, spinocerebellar ataxia 29, spinocerebellar ataxia 42, spinocerebellar ataxia 3, spinocerebellar ataxia (autosomal recessive 1), spinocerebellar ataxia (autosomal recessive 3), spinocerebellar ataxia (autosomal recessive 4), spinocerebellar ataxia (autosomal recessive 5), spinocerebellar ataxia (autosomal recessive, with axonal neuropathy), spinocerebellar ataxia (Machado-Joseph type II), spinocerebellar ataxia (X-linked, 2), spinocerebellar ataxia (X-linked, 3), spinocerebellar ataxia (X-linked, 4), spinocerebellar degenerescence (book type), stroke (e.g., acute or hemorrhagic), vertebral artery dissection, vertebral-basilar insufficiency, and diseases caused by vitamin deficiencies, among a variety of others. In one embodiment, the ataxia is the result of a disease selected from Spinocerebellar ataxia, Friedriech's ataxia, and fragile X/tremor ataxia syndrome. In another particular embodiment, the ataxia is the result of Spinocerebellar ataxia or fragile X/tremor ataxia syndrome.

### Tinnitus

Methods of treating tinnitus in a subject in need thereof are provided herein and comprise administering a dosage form or composition as disclosed herein. Tinnitus is a condition in which those affected perceive sound in one or both ears or in the head when no external sound is present. Often referred to as “ringing” in the ears, tinnitus can occur intermittently or consistently with a perceived volume ranging from low to painfully high. However, the perceived volume of tinnitus can vary from patient to patient where an objective measure of tinnitus volume in one patient may be perceived as painful but, in another patient, the same volume may be perceived as subtle.

### Sleep Disorders

Methods of treating or preventing sleep disorder (e.g., narcolepsy) comprising administering a dosage or composition disclosed herein are provided herein. For example, a sleep disorder may be a central disorder of hypersomnolence, narcolepsy type I, narcolepsy type II, idiopathic hypersomnia, Kleine-Levin syndrome, hypersomnia due to a medical disorder, hypersomnia due to a medication or substance, hypersomnia associated with a psychiatric disorder, insufficient sleep syndrome, circadian rhythm sleep-wake disorders, delayed sleep-wake phase disorder, advanced sleep-wake phase disorder, irregular sleep-wake rhythm, non-24-hour sleep-wake rhythm disorder, shift work disorder, jet lag disorder, or circadian rhythm sleep-wake disorder not otherwise specified (NOS).

### Combination Therapy

A dosage form or composition described herein (e.g., for use in modulating a T-type calcium ion channel) may be administered in combination with at least one other agent or therapy. A subject to be administered a compound disclosed herein may have a disease, disorder, or condition, or a symptom thereof, that would benefit from treatment with another agent or therapy. In certain embodiments, these diseases or conditions can relate to epilepsy or an epilepsy syndrome (e.g., absence seizures, juvenile myoclonic epilepsy, or a genetic epilepsy) or tremor (e.g., essential tremor).

### Antiepilepsy Agents

Anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, and zonisamide.

### Analgesics

Analgesics are therapeutic agents that are used to relieve pain. Examples of analgesics include opiates and morphinomimetics, such as fentanyl and morphine; paracetamol; NSAIDs, and COX-2 inhibitors. Given the ability of the compounds disclosed herein to treat pain via inhibition of T-type calcium channels (e.g., Cav3.1, Cav3.2, and Cav3.3), combination with analgesics is particularly envisioned.

### Tremor Medications

Tremor medications include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide.

### EXAMPLES

In order that the embodiments described herein may be more fully understood, the following examples are set forth. The synthetic and biological examples described in this application are offered to illustrate the compounds, pharmaceutical compositions, and methods provided herein and are not to be construed in any way as limiting their scope.

### Example 1: a Phase 1, Randomized, Open-Label Trial to Evaluate the Pharmacokinetics, Relative Bioavailability, Safety, Tolerability, and Food Effect of 2 PRAX-944 Modified Release Tablet Formulations in Healthy Adults

PRAX-944 is a small molecule selective inhibitor of T-type calcium channels in development for the treatment of essential tremor (FIG. 1). PRAX-944 is also in development for the treatment of Parkinson's disease. The development of a more convenient dosage form prior to late-stage studies is critical to program success. Minimizing the total number of dosage units (e.g., tablets) needed to deliver specific dose levels and and tailoring the physical size and shape of the tablet to make it easier to grasp may be more convenient and usable for patients, especially those with essential tremor (ET). FIG. 2A shows tablets of various shapes and sizes, and FIG. 2B shows a comparison between the currently available PRAX-944 dosage form, which is a modified release (MR) formulation available as 5-mg and 20-mg tablets that are round and small in size (6 mm in diameter), and an exemplary bioequivalent single-unit dosage form described herein. The purpose of this trial is to evaluate the pharmacokinetics (PK) of new PRAX-944 tablets designed to be more convenient for patients.

The current PRAX-944 dosage form is a modified release (MR) formulation available as 5-mg and 20-mg tablets that are round and small in size (6 mm in diameter). The current maximum planned clinical dose is up to 120 mg. This 120 mg dose level requires study participants to take 6 small, round, 20-mg tablets of PRAX-944.

In some instances, titration is required to reach doses >40 mg. The current PRAX-944 titration schedule uses 20 mg, 40 mg, and 80 mg as interim dose levels to reach the current maximum planned dose of 120 mg. Each dose level above 20 mg requires participants to take multiple 20-mg tablets. The new tablets used in the current trial will be larger than those used in previous trials and will include single-unit dose strengths for 20 mg, 40 mg, 80 mg, and 120 mg.

This trial will evaluate the PK of the new, larger capsule-shaped single-unit dose strength MR tablets (15 mm in length and 6.5 mm in width) compared to the current, small, round 20-mg tablets at each of the dose levels used in the current titration regimen: 20 mg, 40 mg, 80 mg, and 120 mg. The primary objective of this relative bioavailability trial will be to compare the rate (maximum plasma drug concentration; Cmax) and extent (area under the plasma concentration-time curve; AUC) of absorption of PRAX-944 from the larger 120-mg tablets versus multiple small, round 20-mg tablets. These parameters are related to the potential safety and efficacy of PRAX-944. Comparisons of other dose levels will also be made.

In a prior study (Z944-103), administration of a single, small, round 20-mg PRAX-944 MR tablet with a high-fat, high-calorie meal resulted in a 14% to 15% increase in AUC and a 49% increase in Cmax compared with administration in a fasted state. No clinically important differences in adverse events (AEs) were noted between the fed and fasted states. This study will further evaluate the food effect of PRAX-944 as administered with the new, larger 120-mg tablet.

### Overall Study Design

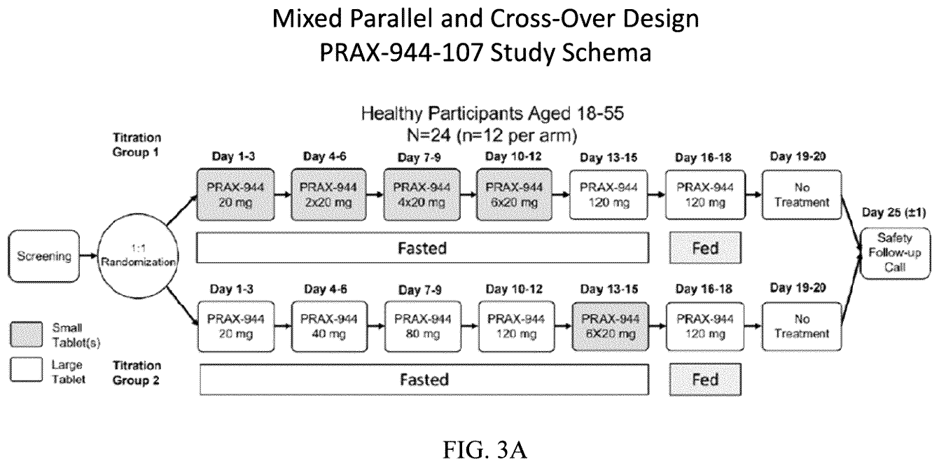

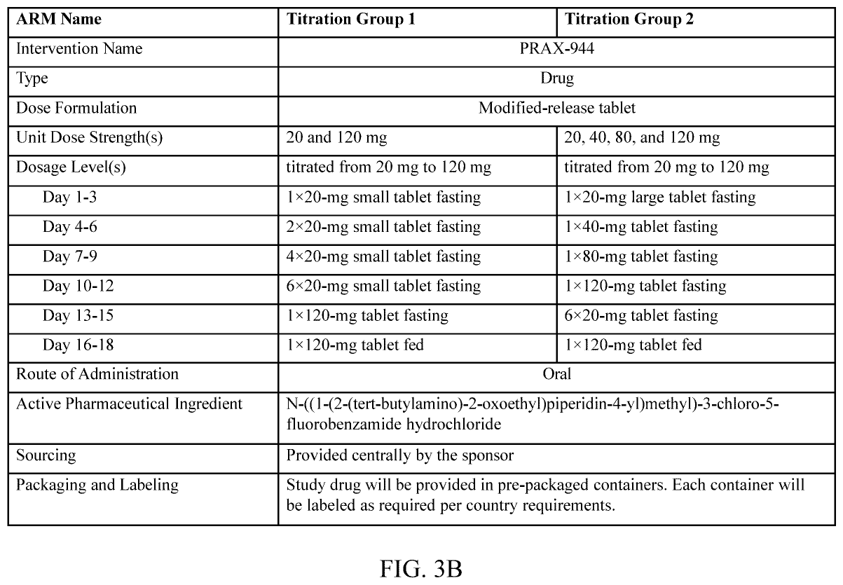

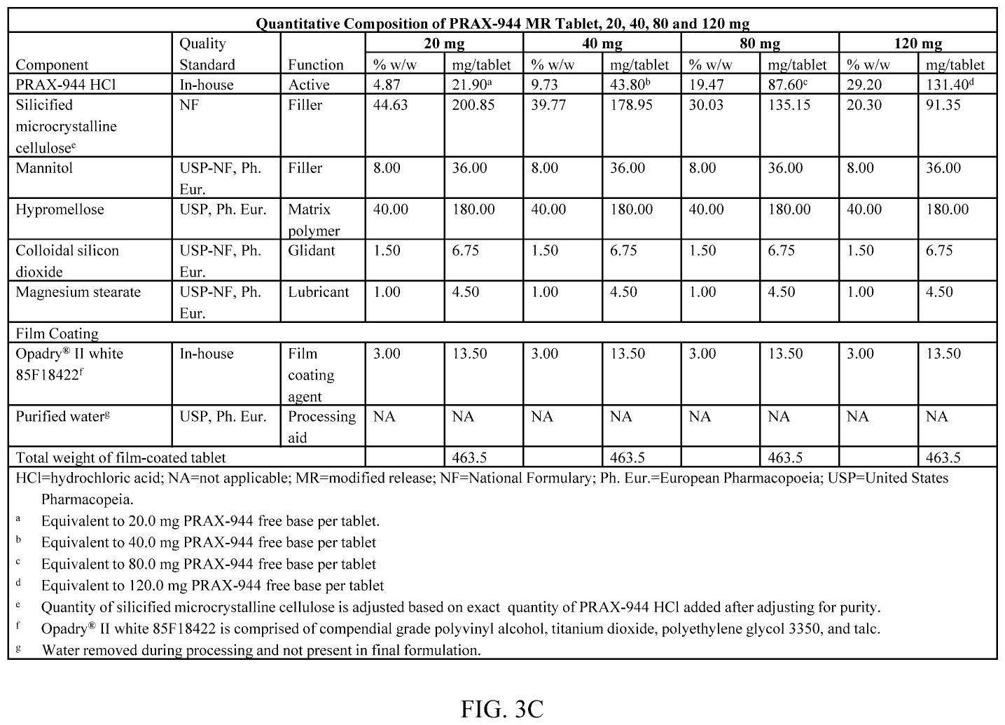

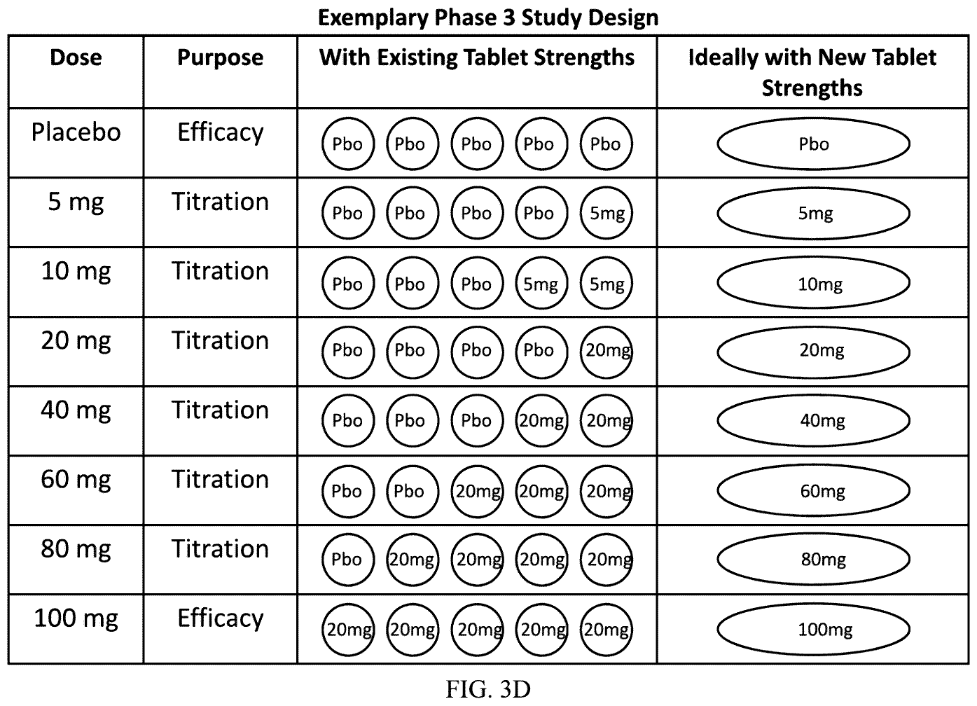

This ongoing single-center, randomized, open-label, Phase 1 relative bioavailability clinical trial will assess the PK, safety, and tolerability of large, single-unit dosage PRAX-944 tablets (20, 40, 80, and 120 mg) compared to the same dose levels delivered with small, round 20-mg PRAX-944 tablets (1, 2, 4, and 6 tablets, respectively) in healthy male or female participants aged 18 to 55 years. Participants will be randomized to titration formulation (i.e., either single-unit or small, round 20-mg tablets) in a 1:1 ratio. After reaching steady state at the 120 mg dose, all participants will then switch to receive the other formulation at the 120 mg dose. Additionally, the effect of food on the PK of the 120-mg single-unit dosage PRAX-944 tablet will be evaluated. Food effect will be assessed at the 120-mg dose level only. Comparisons between dosage forms at the 120-mg dose level will be within-participant comparisons, as will comparisons between the fed and fasted states (FIG. 3A). Comparisons between dosage forms at the 20-mg, 40-mg, and 80-mg dose levels will be between-group comparisons.

The test products are large, single-unit dosage PRAX-944 tablets (20 mg, 40 mg, 80 mg, and 120 mg) and the reference therapy is the small, round 20-mg PRAX-944 tablet.

An overview of the trial design and tablet formulations is shown in FIGS. 3A-3C. A summary of the trial objectives and endpoints is shown in Table 2, the study drugs administered is shown in Table 3, and tablet formulation is shown in Table 4. The trial will consist of 3 periods: Screening/Baseline, Intervention, and Safety Follow-up. Complete details for required assessments and PK analysis timepoints are provided in the Schedule of Activities (SoA; Table 5). FIG. 3D depicts a schematic of an exemplary Phase 3 clinical trial study design to evaluate the efficacy of the large, single-unit dosage PRAX-944 tablets (20 mg, 40 mg, 80 mg, and 120 mg) compared to the same dose levels delivered with the small, round 20-mg PRAX-944 tablets (1, 2, 4, and 6 tablets, respectively).

TABLE 2
Objectives and Endpoints

| | |
|---|---|
| Objective | Endpoint |

Primary

| | |
|---|---|
| To evaluate the plasma pharmacokinetics (PK) | Plasma concentrations of PRAX-944 |
| of PRAX-944, including relative bioavailability, | Maximum plasma drug concentration |
| following administration of 20, 40, 80, and 120 | (Cₘₐₓ) |
| mg using large, single-unit dosage PRAX-944 | Time to reach maximum plasma drug |
| tablets and small, round 20-mg PRAX-944 | concentration following drug |
| tablets | administration (tₘₐₓ) |
| | Area under the plasma concentration- |
| | time curve during a dosage interval |
| | (AUC_{τ}) |
| | Geometric mean ratio between large, |
| | single- unit dosage PRAX-944 tablets |
| | and the same dose levels delivered with |
| | small, round 20-mg PRAX-944 tablets |
| | for PRAX-944 Cₘₐₓ and AUC_{τ} |
| To evaluate the effect of food on the plasma | Plasma concentrations of PRAX-944 at |
| PK of PRAX-944 following administration of | the 120-mg dose level under fed and |
| the 120-mg single-unit dosage PRAX-944 | fasted conditions |
| tablet | Geometric mean ratio between fed and |
| | fasted states for Cₘₐₓ and AUC_{τ} |

Secondary

| | |
|---|---|
| To evaluate the safety and tolerability of PRAX- | Incidence and severity of adverse events |
| 944 in healthy participants at all dose levels | (AEs) |
| when administered as 2 different unit dosages | Changes in vital sign measurements |
| and following administration of the 120-mg | Changes in clinical laboratory results |
| single unit dosage PRAX-944 tablet in the fed | Changes in electrocardiogram (ECG) |
| versus fasted states | measures |
| | Incidence of Columbia-Suicide |
| | Severity Rating Scale (C-SSRS) |
| | measured suicidal ideation or |
| | behavior |

Exploratory

| | |
|---|---|
| To evaluate the plasma PK of | Plasma concentrations of |
| metabolites of PRAX-944 | metabolites of PRAX-944 |
| | Cₘₐₓ |
| | tₘₐₓ |
| | AUC_{τ} |

TABLE 3
Study Drugs(s) Administered

| ARM Name | Titration Group 1 | Titration Group 2 |
|---|---|---|
| Intervention Name | PRAX-944 | |
| Type | Drug | |
| Dose Formulation | Modified-release tablet | |
| Unit Dose Strength(s) | 20 and 120 mg (small and round) | 20, 40, 80, and 120 mg |
| Dosage Level(s) | titrated from 20 mg to 120 mg | titrated from 20 mg to 120 mg |
| Day 1-3 | 1 × 20-mg small tablet fasting | 1 × 20-mg large tablet fasting |
| Day 4-6 | 2 × 20-mg small tablet fasting | 1 × 40-mg tablet fasting |
| Day 7-9 | 4 × 20-mg small tablet fasting | 1 × 80-mg tablet fasting |
| Day 10-12 | 6 × 20-mg small tablet fasting | 1 × 120-mg tablet fasting |
| Day 13-15 | 1 × 120-mg tablet fasting | 6 × 20-mg tablet fasting |
| Day 16-18 | 1 × 120-mg tablet fed | 1 × 120-mg tablet fed |

| | |
|---|---|
| Route of Administration | Oral |
| Active Pharmaceutical | N-((1-(2-(tert-butylamino)-2-oxoethyl)piperidin-4- |
| Ingredient | yl)methyl)-3-chloro-5-fluorobenzamide hydrochloride |
| Sourcing | Provided centrally by the sponsor |
| Packaging and Labeling | Study drug will be provided in pre-packaged containers. Each |
| | container will be labeled as required per country requirements. |

TABLE 4
PRAX-944 MR Tablets
Quantitative Composition of PRAX-944 MR Tablet, 20, 40, 80 and 120 mg

| | Quality | | 20 mg | | 40 mg | | 80 mg | | 120 mg | |
|---|---|---|---|---|---|---|---|---|---|---|
| Component | Standard | Function | % w/w | mg/tablet | % w/w | mg/tablet | % w/w | mg/tablet | % w/w | mg/tablet |
| PRAX-944 HCl | In-house | Active | 4.87 | 21.90^{a} | 9.73 | 43.80^{b} | 19.47 | 87.60^{c} | 29.20 | 131.40^{d} |
| Silicified | NF | Filler | 44.63 | 200.85 | 39.77 | 178.95 | 30.03 | 135.15 | 20.30 | 91.35 |
| microcrystalline | | | | | | | | | | |
| cellulose^{e} | | | | | | | | | | |
| Mannitol | USP-NF, Ph. | Filler | 8.00 | 36.00 | 8.00 | 36.00 | 8.00 | 36.00 | 8.00 | 36.00 |
| | Eur. | | | | | | | | | |
| Hypromellose | USP, Ph. | Matrix | 40.00 | 180.00 | 40.00 | 180.00 | 40.00 | 180.00 | 40.00 | 180.00 |
| | Eur. | polymer | | | | | | | | |
| Colloidal | USP-NF, Ph. | Glidant | 1.50 | 6.75 | 1.50 | 6.75 | 1.50 | 6.75 | 1.50 | 6.75 |
| silicon dioxide | Eur. | | | | | | | | | |
| Magnesium | USP-NF, Ph. | Lubricant | 1.00 | 4.50 | 1.00 | 4.50 | 1.00 | 4.50 | 1.00 | 4.50 |
| stearate | Eur. | | | | | | | | | |

Film Coating

| | | | | | | | | | | |
|---|---|---|---|---|---|---|---|---|---|---|
| Opadry ® II | In-house | Film | 3.00 | 13.50 | 3.00 | 13.50 | 3.00 | 13.50 | 3.00 | 13.50 |
| white | | coating | | | | | | | | |
| 85F18422^{f} | | agent | | | | | | | | |
| | USP, Ph. | Processing | NA | NA | NA | NA | NA | NA | NA | NA |
| Purified water^{g} | Eur. | aid | | | | | | | | |

| | | | | |
|---|---|---|---|---|
| Total weight of film-coated tablet | 463.5 | 463.5 | 463.5 | 463.5 |

HCl = hydrochloric acid; NA = not applicable; MR = modified release; NF = National Formulary; Ph. Eur. = European Pharmacopoeia; USP = United States Pharmacopeia.
^{a}Equivalent to 20.0 mg PRAX-944 free base per tablet.
^{b}Equivalent to 40.0 mg PRAX-944 free base per tablet
^{c}Equivalent to 80.0 mg PRAX-944 free base per tablet
^{d}Equivalent to 120.0 mg PRAX-944 free base per tablet
^{e}Quantity of silicified microcrystalline cellulose is adjusted based on exact quantity of PRAX-944 HCl added after adjusting for purity.
^{f}Opadry ® II white 85F18422 is comprised of compendial grade polyvinyl alcohol, titanium dioxide, polyethylene glycol 3350, and talc.
^{g}Water removed during processing and not present in final formulation.

### Intervention Period

Approximately 24 healthy male and female participants between the ages of 18 and 55 years with body mass index (BMI) between 18 and 32 kg/m2 and body weight of at least 50 kg are planned for enrollment. Participants will be randomized in a 1:1 ratio to receive dose titration with either small, round 20-mg PRAX-944 tablets (Titration Group 1) or large, single-unit dosage PRAX-944 tablets (Titration Group 2). Formulation will not be blinded. The dose levels to be administered in both formulation groups are 20 mg, 40 mg, 80 mg, and 120 mg; see FIG. 3A for complete titration regimen. On Days 1 to 15, all participants will receive PRAX-944 in the morning after a 10-hour overnight fast. Participants are to remain fasted for 4 hours post dose.

Participants will receive the same dosage form (small, round 20-mg PRAX-944 tablets in Titration Group 1 and large, single-unit dosage PRAX-944 tablets in Titration Group 2) on Days 1 to 12. On Day 13 (after participants have received 120 mg PRAX-944 for 3 days), Titration Group 1 will cross over to receive 120 mg PRAX-944 as a large, single-unit dosage PRAX-944 tablet, and participants in Titration Group 2 will cross over to receive 120 mg PRAX-944 as 6 small, round 20-mg tablets. This 120-mg PRAX-944 dose will be administered for 3 days in a fasted state.

On Day 16, all participants will receive 120 mg as a single-unit dosage PRAX-944 tablet in a fed state. Dosing in a fed state will last 3 days (Day 16 to Day 18). On these days, all participants will then receive the 120-mg single-unit dosage PRAX-944 tablet co-administered with a high-fat, high-calorie meal. Participants will be observed in the research unit for 2 days after the last dose of study drug.

### Pharmacokinetic Assessments

Pharmacokinetics will be assessed with serial plasma samples collected on the third day of dosing at each dose level (Days 3, 6, 9, 12, 15, and 18). This schedule allows for the calculation of AUC_{τ} at steady state. A validated bioanalytical method will be utilized for the determination of plasma concentrations of PRAX-944 and its metabolites.

Relative bioavailability evaluations will compare the PK between groups for the 20-(Day 3), 40-(Day 6), and 80-mg (Day 9) dose levels and within participants for the 120-mg dose level (Day 12 versus Day 15). The food effect comparison will also be within participants (Day 15 versus Day 18 in Titration Group 1 and Day 12 versus Day 18 in Titration Group 2).

Samples collected may also be used to evaluate safety aspects related to concerns arising during or after the clinical trial, for additional exploratory method development, and for metabolite characterization.

### Pharmacokinetic Analyses

Blood samples will be collected for measurement of plasma concentrations of PRAX-944 and its metabolites. PK will be assessed with serial plasma samples collected on the third day of dosing at each dose level (Days 3, 6, 9, 12, 15, and 18). This schedule allows for the calculation of AUC_{τ} at steady state. A validated bioanalytical method will be utilized for the determination of plasma concentrations of PRAX-944 and its metabolites.

Plasma concentrations will be summarized by treatment condition and timepoint using descriptive statistics. In addition to descriptive statistics specified above, plasma concentrations will also be summarized using number and percentage of concentrations below the limit of quantification and coefficient of variation (CV %).

Pharmacokinetic parameters will be estimated from concentration-time data using noncompartmental methods for PRAX-944, M15, M23, and M32 in plasma.

Pharmacokinetic parameters will be summarized by treatment condition using descriptive statistics. In addition to the descriptive statistics specified above, PK parameters will also be summarized using geometric mean and geometric CV %. Geometric mean and geometric CV % will not be presented for tₘₐₓ.

Relative bioavailability evaluations will compare the PK between groups for the 20-mg (Day 3), 40-mg (Day 6), and 80-mg (Day 9) dose levels and within participants for the 120-mg dose level (Day 12 versus Day 15). The food effect comparison will also be within participants (Day 15 versus Day 18 in Titration Group 1 and Day 12 versus Day 18 in Titration Group 2).

For the assessment of relative bioavailability and food effect, there are no predefined acceptance criteria; this trial is intended to compare exposure of the test treatment relative to the reference treatment.

To evaluate relative bioavailability of the 2 PRAX-944 120-mg formulations, separate mixed-effects models will be fitted to AUC_{τ} and Cₘₐₓ values. Each PK parameter will be natural log transformed prior to analysis. For each parameter, the mixed-effects model will include sequence, period, and titration formulation as fixed effects and participant within sequence as a random effect. The geometric mean ratio (In transformed) of the test treatment (i.e., larger, single-unit dose strength PRAX-944 tablets) relative to the reference treatment (i.e., smaller, round 20-mg PRAX-944 tablets) along with 90% confidence interval will be estimated for AUC_{τ} and Cₘₐₓ.

To evaluate the relative bioavailability of the large, single-unit 120-mg PRAX-944 tablet formulation between the fed and fasted states, separate mixed effects models will be fitted to AUC_{τ} and Cₘₐₓ values. Each PK parameter will be natural log transformed prior to analysis. For each parameter, the mixed effects model will include treatment condition (i.e., fed versus fasted) as a fixed effect and participant as a random effect.

To evaluate the between-participant relative bioavailability of doses under 120 mg, mixed effects models will be fitted to AUC_{τ} and Cₘₐₓ values on Days 3, 6, and 9. Each PK parameter will be natural log transformed prior to analysis. For each parameter, the mixed effects model will include titration formulation, day, and titration formulation by day interaction as fixed effects and participant as a random effect. The fixed effect for day is synonymous with the dose level used based on the studied dose titration schedule (i.e., 20 mg on Day 3, 40 mg on Day 6, etc.). The geometric mean ratio (In transformed) of the test treatment (i.e., larger, single-unit dose strength PRAX-944 tablets) relative to the reference treatment (i.e., smaller, round 20-mg PRAX-944 tablets) along with 90% confidence interval will be estimated for AUC_{τ} and Cₘₐₓ at each day.

### Scientific Rationale for Clinical Trial Design

Since the trial is comparing the new, larger, single-unit dose strength MR tablets (15 mm in length and 6.5 mm in width) delivering the same dose levels achieved using the requisite number of smaller, round 20-mg tablets (1, 2, 4, and 6 tablets, respectively), the study must be an open-label design. This study will be randomized to ensure an unbiased assignment to 1 of 2 titration formulation arms.

### Justification for Dose

The current PRAX-944 titration schedule uses 20 mg, 40 mg, and 80 mg as interim dose levels to reach the current maximum planned dose of 120 mg. Each dose level above 20 mg requires participants to take multiple 20-mg tablets. The new tablets used in the current trial will be larger than those used in previous trials and will include single-unit dose strengths for 20 mg, 40 mg, 80 mg, and 120 mg.

### Inclusion Criteria

Participants must meet all of the following criteria at Screening and Admission, unless otherwise specified, to be eligible to participate in this clinical trial:
I 1. Willing and able to provide informed consent indicating that they understand the purpose of the clinical trial and the procedures that are required for the clinical trial, and they are willing to comply with scheduled visits and all trial-related procedures
I 2. Male or female between the ages of 18 and 55 years
I 3. Weight of at least 50 kg with BMI between 18 and 32 kg/m²
I 4. Female of non-childbearing potential, non-pregnant female of childbearing potential who is willing to use adequate methods of contraception as defined in this clinical trial protocol, or male who is willing and able to use adequate methods of contraception as defined in this clinical trial protocol
I 5. Suitable venous access for blood sampling
I 6. In good health, determined by no clinically significant findings from medical history, physical examination, 12-lead ECG, vital sign measurements, or clinical laboratory evaluations

Evaluations needed to determine eligibility that require central processing or are not conducted at Admission will not be used to determine eligibility at Admission.

### Exclusion Criteria

A participant who meets any of the following criteria (at Screening or Admission unless otherwise specified) will be excluded from this clinical trial:
E 1. Known allergy or hypersensitivity to any component of the formulation of PRAX-944 or history of severe allergy or anaphylaxis to a drug, food, or other exposure
E 2. Any clinically significant arrhythmia(s) on ECG; specifically, the participant's corrected QT interval (Fridericia's correction) is >450 ms for males or >470 ms for females
E3 Any abnormal laboratory value or physical examination finding that is judged by the investigator as clinically significant in consultation with the sponsor
E 4. Any of the following: a serum total bilirubin value >1.5×upper limit of normal (ULN) or a serum alanine aminotransferase (ALT) or aspartate aminotransferase (AST) value >2×ULN
E 5. History of stomach or intestinal surgery or resection that would potentially alter absorption and/or excretion of orally administered drugs (uncomplicated appendectomy and hernia repair will be allowed; cholecystectomy is not allowed)
E 6. Hemoglobin <the lower limit of normal (LLN)
E 7. Serology test positive for human immunodeficiency virus (HIV) or hepatitis B or C
E 8. Positive urine test for ethanol
E 9. Positive urine test for barbiturates, cocaine, methamphetamines, methadone, benzodiazepines, phencyclidine, tetrahydrocannabinols, methylenedioxymethamphetamine, opiates, or amphetamines
E 10. Smokers, those who use vaping products, or positive urine cotinine test
E 11. Use of systemic prescription medications, excluding hormonal contraception; over-the-counter medication, including multivitamins; and dietary and herbal supplements within 1 week or 5 times the terminal half-life of the medication prior to the first dose of study drug (whichever is longer) and for the duration of the trial
E 12. Consumption of grapefruit, grapefruit juice, and Seville oranges within 2 weeks prior to the first dose of study drug until discharge from the clinic
E 13. Use of any experimental or investigational drug or device within 30 days prior to the first dose of study drug or 5 times the terminal half-life of the drug, whichever is longer Blood donation (excluding plasma donations) of approximately 1 pint (500 mL) or E 14. more within 60 days prior to the first dose of study drug
E 15. History of epilepsy or a previous history of seizures (including febrile seizures)
E 16. History of substance use disorder consistent with Diagnostic and Statistical Manual of Mental Disorders (DSM-5) criteria, in the opinion of the investigator
E 17. History of any suicide attempt or a history of suicidal ideation within the last 5 years
E 18. History of cancer, except for non-melanoma skin cancer resected >2 years ago and cervical intraepithelial neoplasia >5 years ago that have been definitively treated and considered cured. Particular attention should be paid to excluding potential participants who have been treated with chemotherapy.
E 19. Participants who are research unit staff members directly involved in the conduct of the clinical trial and their family members, research unit staff members otherwise supervised by the investigator, or participants who are sponsor employees directly involved in the conduct of the clinical trial
E 20. Ongoing or history of any medical (including, but not limited to, heart disease, stroke and diabetes), psychiatric (including, but not limited to, moderate to severe depression, bipolar affective disorder, and psychotic illness), or surgical condition that, in the judgment of the investigator, might jeopardize the participant's safety; confound the trial results; or interfere with the absorption, distribution, metabolism or excretion of the study drug
E 21. Any vaccination within 28 days of the first dose of study drug E 21.

Evaluations needed to determine eligibility that require central processing or are not conducted at Admission will not be used to determine eligibility at Admission.

### Meals and Dietary Restrictions

On Days 1 to 15, all participants will receive PRAX-944 in the morning after an overnight fast (no food or drink, except water, will be allowed for at least 10 hours prior to dosing). Participants are to remain fasted for 4 hours post dose.

On Day 16, all participants will receive 120 mg as a single-unit dosage PRAX-944 tablet in a fed state. Dosing in a fed state will last 3 days (Day 16 to Day 18). On these days, all participants will then receive the 120-mg single-unit dosage PRAX-944 tablet co-administered with a high-fat, high-calorie meal.

As stated in the exclusion criteria, participants must avoid consumption of grapefruit, grapefruit juice, and Seville oranges within 2 weeks prior to first dose of study drug until discharge from the clinic. Caffeine containing foods and beverages will not be allowed for 48 hours before Admission (Day-1) until Discharge (Day 20; refer to SoA, Table 5).

### Results

Results, to date, for this ongoing single-center, randomized, open-label, Phase 1 relative bioavailability clinical trial assessing the PK, safety, and tolerability of large, single-unit dosage PRAX-944 tablets (20 mg, 40 mg, 80 mg, and 120 mg) compared to the same dose levels delivered with small, round 20-mg PRAX-944 tablets (1, 2, 4, and 6 tablets, respectively) in healthy participants demonstrate that the large, single-unit dosage PRAX-944 tablets are bioequivalent to the small, round 20-mg PRAX-944 tablets.

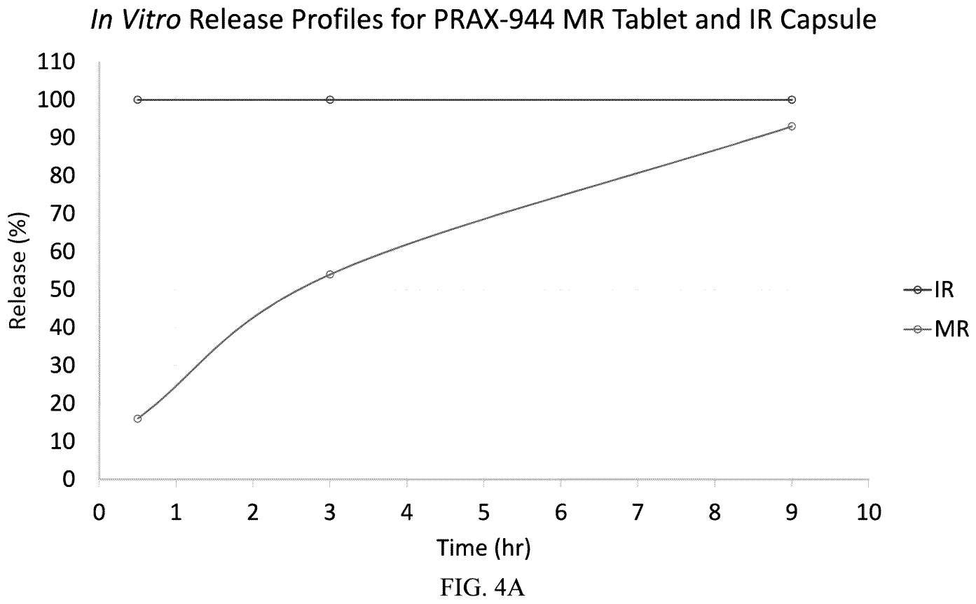

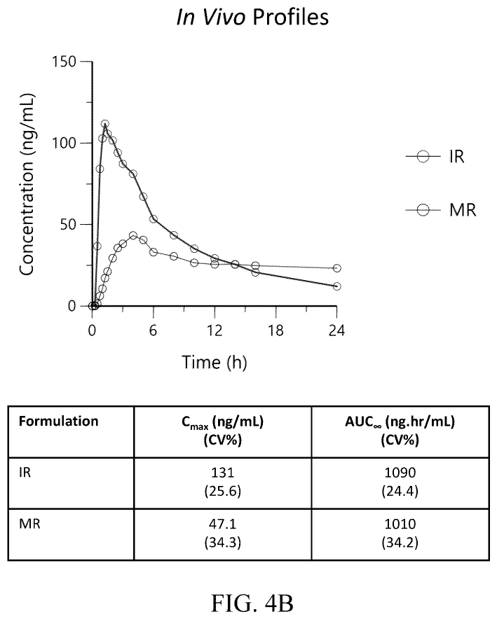

For improved tolerability, PRAX-944 is formulated as a modified release (MR) tablet to extend drug release and absorption, and thus, blunt Cₘₐₓ and prolong plasma concentration-time profile compared to an immediate release (IR) dosage form. In vitro and in vivo release profiles for 20 mg strength PRAX-944 modified release (MR) tablet and immediate release (IR) capsule are shown in FIGS. 4A-4B. As shown in FIG. 4A, about 100% of the PRAX-944 is released within about 1 hour for the immediate release (IR) dosage form; and about 80% of the PRAX-944 is released within about 7 hours for the modified release (MR) dosage form. As shown in FIG. 4B, the IR formulation achieved a Cₘₐₓ (ng/ml) max (CV %) of 131 (25.6) and an AUC_{∞} (ng·hr/mL) (CV %) of 1090 (24.4); and the MR formulation achieved a Cₘₐₓ (ng/mL) (CV %) of 47.1 (34.3) and an AUC_{∞} (ng·hr/mL) (CV %) of 1010 (34.2).

Different PRAX-944 large tablet prototype formulations were evaluated for bioequivalence to the small, round 20-mg PRAX-944 tablets. Indeed, formulation of a successful large tablet provides a beneficial and unexpected result.

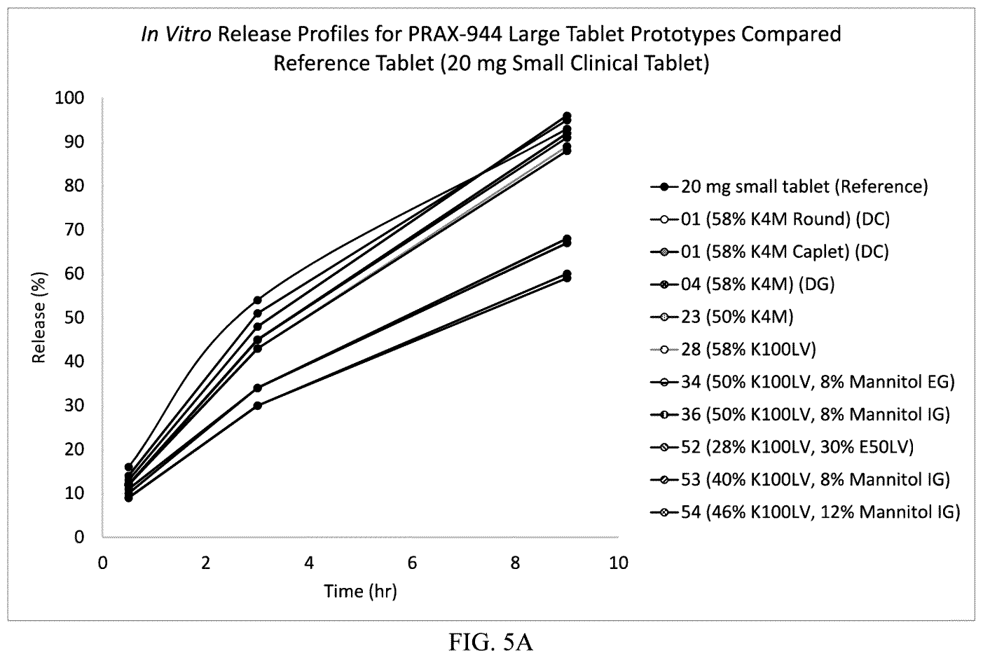

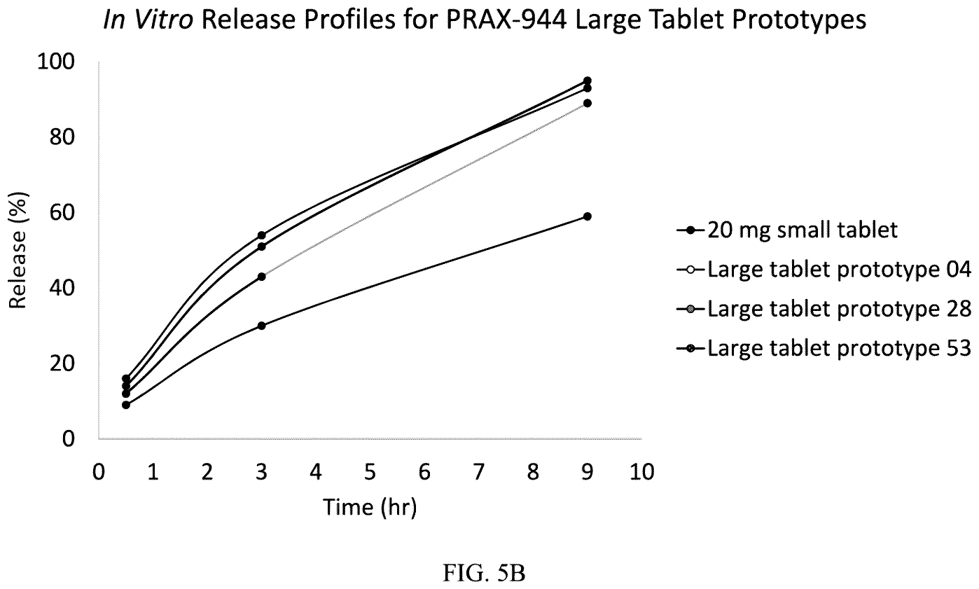

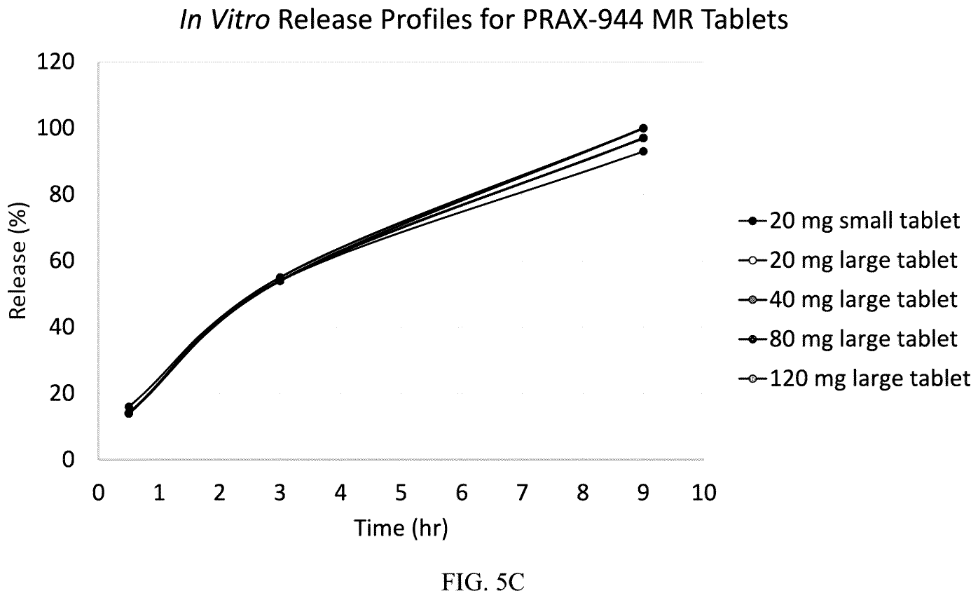

FIG. 5A depicts in vitro release profiles for PRAX-944 large tablet prototypes (i.e., large tablet prototype 01 (58% K4M Round) (DC), large tablet prototype 01 (58% K4M Caplet) (DC), large tablet prototype 04 (58% K4M) (DG), large tablet prototype 23 (50% K4M), large tablet prototype 28 (58% K100LV), large tablet prototype 34 (50% K100LV, 8% Mannitol EG), large tablet prototype 36 (50% K100LV, 8% Mannitol IG), large tablet prototype 52 (28% K100LV, 30% E50LV), large tablet prototype 53 (40% K100LV, 8% Mannitol IG), and large tablet prototype 54 (46% K100LV, 12% Mannitol IG)) compared to reference tablet (i.e., 20 mg small, round clinical tablet). DC and DG designate direct compression and dry granulation, respectively. FIG. 5B depicts in vitro release profiles for PRAX-944 large tablet prototypes (i.e., large tablet prototype 04, large tablet prototype 28, and large tablet prototype 53) compared to reference tablet (i.e., 20 mg small, round clinical tablet). FIG. 5C depicts in vitro release profiles for PRAX-944 MR tablets (i.e., 20 mg large tablet, 40 mg large tablet, 80 mg large tablet, and 120 mg large tablet) compared to reference tablet (i.e., 20 mg small, round clinical tablet). Together these data demonstrate that the large, single-unit dosage PRAX-944 tablets (20 mg, 40 mg, 80 mg, and 120 mg) are bioequivalent across dose levels with the small, round 20-mg PRAX-944 tablets (1, 2, 4, and 6 tablets, respectively). Additional properties of the PRAX-944 large tablet prototypes are described in Tables 6-16.

TABLE 6
Prototype #01 (58% K4M Round) (Direct Compression
(DC)) Round (Punches: 11.0 mm Round SC)

| Ingredients | % w/w | mg/tab. |
|---|---|---|
| PRAX 944 HCl | 29.20 | 131.40 |
| MCC (Avicel PH 102) | 10.30 | 46.35 |
| Methocel K4M Premium DC2 | 58.00 | 261.00 |
| Colloidal silicon dioxide (Aerosil 200) | 1.50 | 6.75 |

Lubrication

| | | |
|---|---|---|
| Mg. stearate | 1.00 | 4.50 |
| Total | 100.00 | 450.00 |

TABLE 7
Prototype #01 (58% K4M Caplet) (DC) (Punches:
15.0 × 6.5 mm caplet SC)

| Ingredients | % w/w | mg/tab. |
|---|---|---|
| PRAX 944 HCl | 29.20 | 131.40 |
| MCC (Avicel PH 102) | 10.30 | 46.35 |
| Methocel K4M Premium DC2 | 58.00 | 261.00 |
| Colloidal silicon dioxide (Aerosil 200) | 1.50 | 6.75 |

Lubrication

| | | |
|---|---|---|
| Mg. stearate | 1.00 | 4.50 |
| Total | 100.00 | 450.00 |

TABLE 8
Prototype #01 (58% K4M) (DC) Caplet - Compression Profiling

| | | | | |
|---|---|---|---|---|
| Machine: Korsch | Punches: 15.0 × | No. of | Feeder: | Press speed: |
| machine | 6.5 mm caplet SC | punches: 02 | Gravity | 20-25 RPM |

| | | |
|---|---|---|
| Average weight | 450 mg ± 50 mg | 450 mg ± 50 mg |
| of tablets | (415 to 475 mg) | (410-488 mg) |
| Thickness (mm) | 5.40-5.56 | 5.19-5.34 |
| | mm | mm |
| Hardness (N) | 92-108N | 121-144N |
| Compression force at | 11-18 | 14-23 |
| observed hardness | KN | KN |

| | |
|---|---|
| Friability (%) | Not performed due to weight variation |
| Observation | Rat holing was observed in the hopper. Weight variation |
| | was observed. Surface erosion on tablets was seen. Also, |
| | compressed tablets lacked mechanical strength. |

TABLE 9
Prototype #04 (58% K4M) (Dry Granulation (Roller compaction) (DG)) Round

| | | | 20 mg | | 5 mg | |
|---|---|---|---|---|---|---|
| | 120 mg | | Earlier clinical batch | | Earlier clinical batch | |
| | | Quantity | | Quantity | | Quantity |
| Ingredients | % w/w | mg/tab | % w/w | mg/tab. | % w/w | mg/tab. |
| PRAX 944 HCl | 29.20 | 131.40 | 20.00 | 20.00 | 5.00 | 5.00 |
| Avicel PH 101 | 10.30 | 46.35 | 19.50 | 19.50 | 34.50 | 34.50 |
| HPMC K4M Premium CR | 58.00 | 261.00 | 58.00 | 58.00 | 58.00 | 58.00 |
| Aerosil 200 | 1.50 | 6.75 | 1.50 | 1.50 | 1.50 | 1.50 |

Pre-RC Lubrication

| | | | | | | |
|---|---|---|---|---|---|---|
| Mg. stearate | 0.50 | 2.25 | 0.50 | 0.50 | 0.50 | 0.50 |

Post Lubrication

| | | | | | | |
|---|---|---|---|---|---|---|
| Mg. stearate | 0.50 | 2.25 | 0.50 | 0.50 | 0.50 | 0.50 |
| Total | 100.00 | 450.00 | 100.00 | 100.00 | 100.00 | 100.00 |

TABLE 10
Prototype #23 (50% K4M) Caplet
(Dry Granulation (Roller compaction))

120 mg

| | | Quantity |
|---|---|---|
| Ingredients | % w/w | mg/tab |
| PRAX 944 HCl | 29.20 | 131.40 |
| Avicel PH 101 | 18.30 | 82.35 |
| HPMC K4M Premium CR | 50.00 | 225.00 |
| Aerosil 200 | 1.50 | 6.75 |

Pre-RC Lubrication

| | | |
|---|---|---|
| Mg. stearate | 0.50 | 2.25 |

Post Lubrication

| | | |
|---|---|---|
| Mg. stearate | 0.50 | 2.25 |
| Total | 100.00 | 450.00 |

TABLE 11
Prototype #28 (58% K100LV) Caplet
(Dry Granulation (Roller compaction))

120 mg

| | | Quantity |
|---|---|---|
| Ingredients | % w/w | mg/tab |
| PRAX 944 HCl | 29.20 | 131.40 |
| Prosolv HD 90 | 10.30 | 46.35 |
| HPMC K100 LV Premium CR | 58.00 | 261.00 |
| Aerosil 200 | 1.50 | 6.75 |

Pre-RC Lubrication

| | | |
|---|---|---|
| Mg. stearate | 0.50 | 2.25 |

Post Lubrication

| | | |
|---|---|---|
| Mg. stearate | 0.50 | 2.25 |
| Total | 100.00 | 450.00 |

TABLE 12
Prototype #34 (50% K100LV, 8% Mannitol
EG) Caplet (Dry Granulation (Roller compaction))

#34

| | | Quantity |
|---|---|---|
| Ingredients | % w/w | mg/tab |
| PRAX 944 HCl | 30.73 | 138.30 |
| Prosolv HD 90 | 8.77 | 39.45 |
| HPMC K100 LV Premium CR | 50.00 | 225.00 |
| Perlitol 100 SD | | |
| Aerosil 200 | 1.50 | 6.75 |

Pre-RC Lubrication

| | | |
|---|---|---|
| Mg. stearate | 0.50 | 2.25 |

Post Lubrication

| | | |
|---|---|---|
| Perlitol 200 SD | 8.00 | 36.00 |
| Mg. stearate | 0.50 | 2.25 |
| Total | 100.00 | 450.00 |

TABLE 13
Prototype #36 (50% K100LV, 8% Mannitol
IG) Caplet (Dry Granulation (Roller compaction))

#36

| | | Quantity |
|---|---|---|
| Ingredients | % w/w | mg/tab |
| PRAX 944 HCl | 30.73 | 138.30 |
| Prosolv HD 90 | 8.77 | 39.45 |
| HPMC K100 LV Premium CR | 50.00 | 225.00 |
| Perlitol 100 SD | 8.00 | 36.00 |
| Aerosil 200 | 1.50 | 6.75 |

Pre-RC Lubrication

| | | |
|---|---|---|
| Mg. stearate | 0.50 | 2.25 |

Post Lubrication

| | | |
|---|---|---|
| Perlitol 200 SD | | |
| Mg. stearate | 0.50 | 2.25 |
| Total | 100.00 | 450.00 |

TABLE 14
Prototype #52 (28% K100LV, 30% E50LV)
Caplet (Dry Granulation (Roller compaction))

120 mg

| | | Quantity |
|---|---|---|
| Ingredients | % w/w | mg/tab |
| PRAX 944 HCl | 30.73 | 138.30 |
| Prosolv HD 90 | 8.77 | 39.45 |
| HPMC K100 LV Premium | 28.00 | 126.00 |
| HPMC E50 LV Premium | 30.00 | 135.00 |
| Aerosil 200 | 1.50 | 6.75 |

Pre-RC Lubrication

| | | |
|---|---|---|
| Mg. stearate | 0.50 | 2.25 |

Post Lubrication

| | | |
|---|---|---|
| Mg. stearate | 0.50 | 2.25 |
| Total | 100.00 | 450.00 |

TABLE 15
Prototype #53 (40% K100LV, 8% Mannitol
IG) Caplet (Dry Granulation (Roller compaction))

76BG(812)53

| | | Quantity |
|---|---|---|
| Ingredients | % w/w | mg/tab |
| PRAX 944 HCl | 30.73 | 138.30 |
| Prosolv HD 90 | 18.77 | 84.45 |
| HPMC K100 LV Premium CR | 40.00 | 180.00 |
| Perlitol 100 SD | 8.00 | 36.00 |
| Aerosil 200 | 1.50 | 6.75 |

Pre-RC Lubrication

| | | |
|---|---|---|
| Mg. stearate | 0.50 | 2.25 |

Post Lubrication

| | | |
|---|---|---|
| Mg. stearate | 0.50 | 2.25 |
| Total | 100.00 | 450.00 |

TABLE 16
Prototype #54 (46% K100LV, 12% Mannitol
IG) Caplet (Dry Granulation (Roller compaction))

76BG(812)54

| | | Quantity |
|---|---|---|
| Ingredients | % w/w | mg/tab |
| PRAX 944 HCl | 30.73 | 138.30 |
| Prosolv HD 90 | 8.77 | 39.45 |
| HPMC K100 LV Premium CR | 46.00 | 207.00 |
| Perlitol 100 SD | 12.00 | 54.00 |
| Aerosil 200 | 1.50 | 6.75 |

Pre-RC Lubrication

| | | |
|---|---|---|
| Mg. stearate | 0.50 | 2.25 |

Post Lubrication

| | | |
|---|---|---|
| Mg. stearate | 0.50 | 2.25 |
| Total | 100.00 | 450.00 |

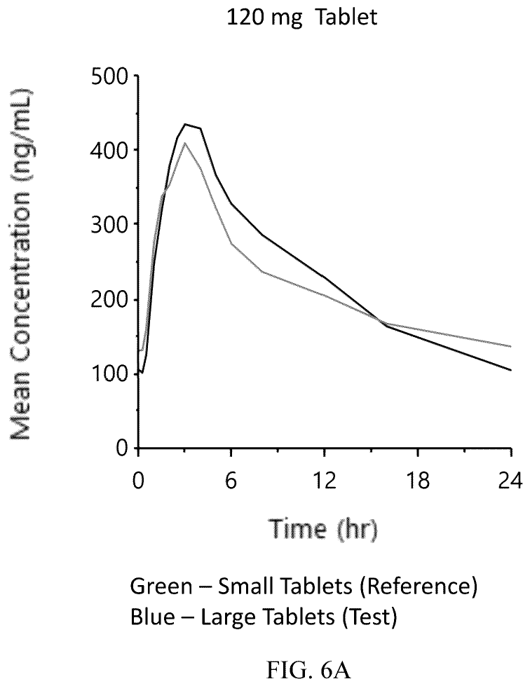

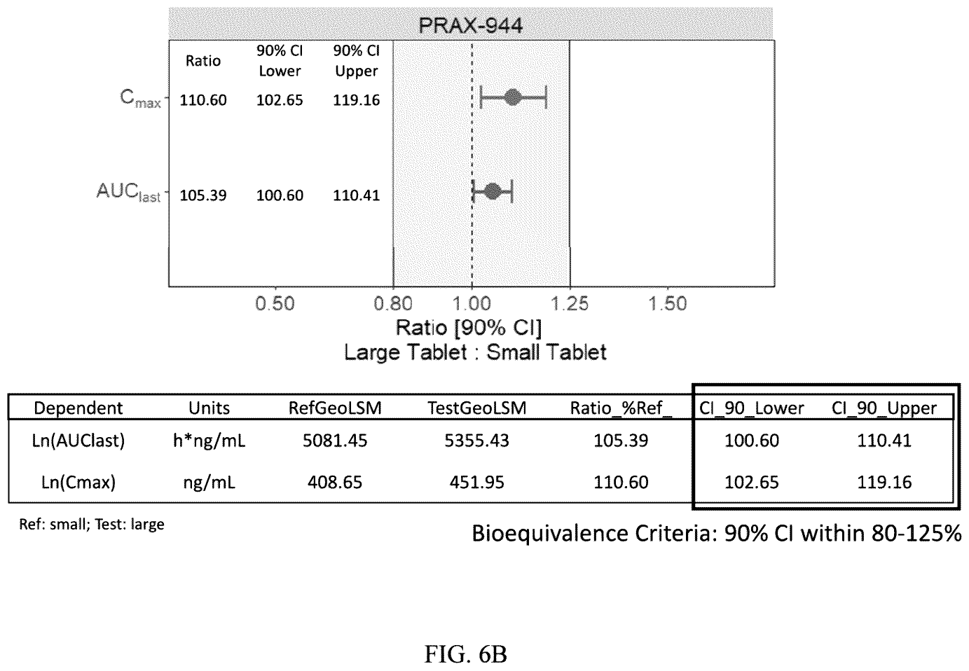

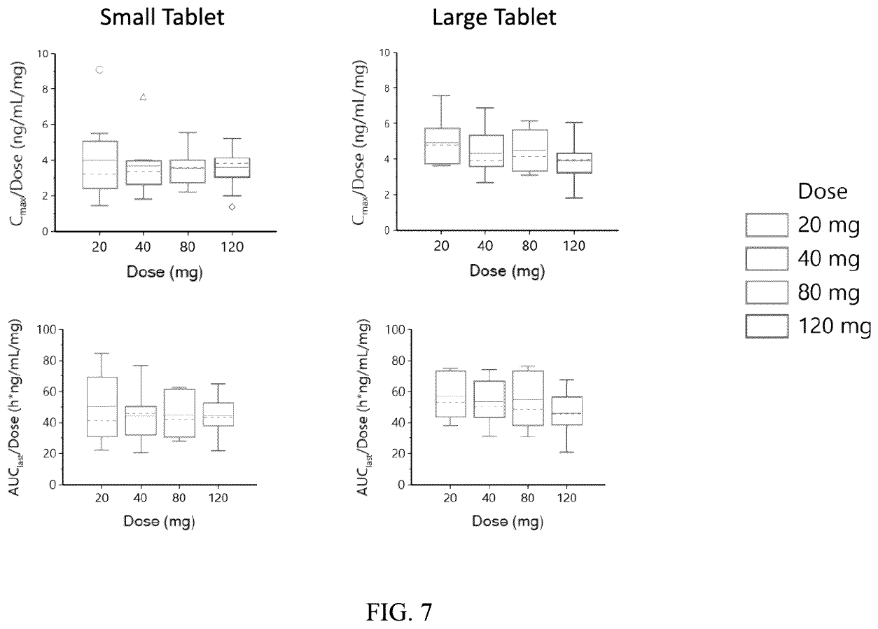

FIGS. 6A-6B depict relative bioavailability of 120 mg large tablets (test) compared to small, round tablets (reference) by cross-over analysis. By crossover analysis, the 120 mg large and small, round tablets meet bioequivalence criteria (90% CI within 80-125%) for AUCₗₐₛₜ and Cₘₐₓ (FIG. 6B). Outlier subjects excluded from analysis.

FIG. 7 depicts dose proportionality with small, round tablets, or large tablets. Outlier subjects excluded from analysis.

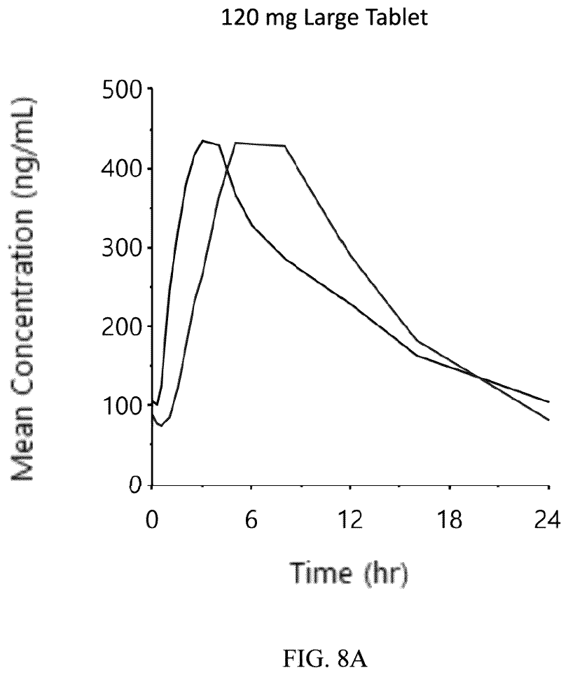

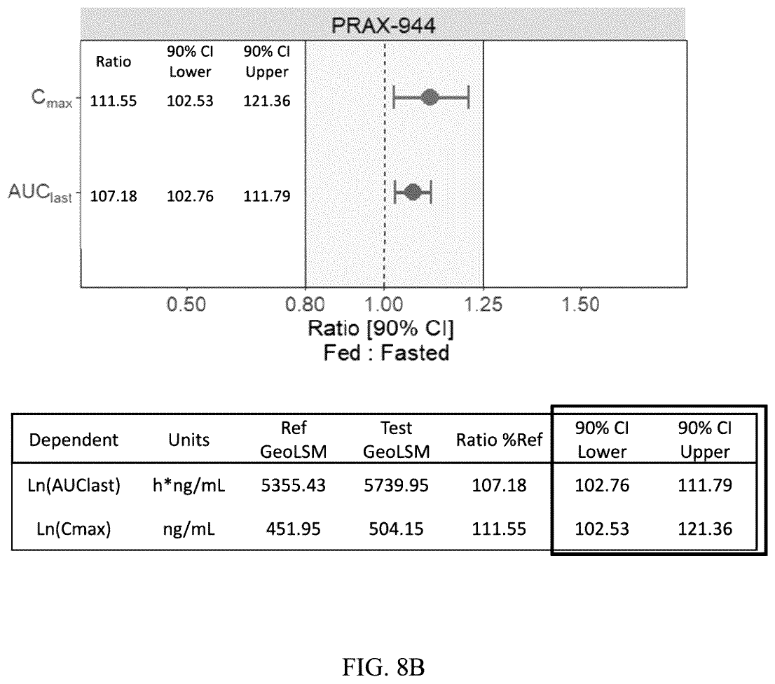

FIGS. 8A-8B depict lack of food effect with 120 mg PRAX-944. Coadministration of the 120 mg large tablet with a high-fat, high-calorie meal results in a delay in tₘₐₓ of 3 hours and a slight increase in AUC and Cₘₐₓ but within the no effect boundaries (90% CI within 80-125%). Outlier subjects excluded from analysis.

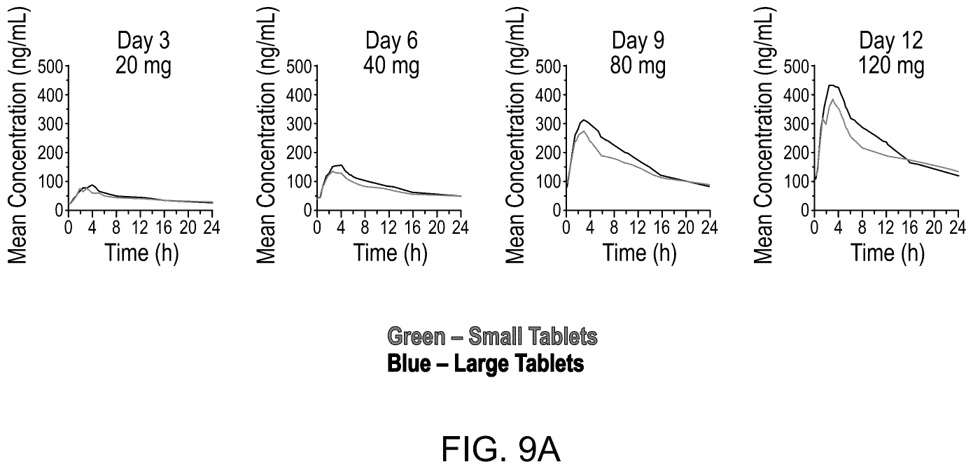

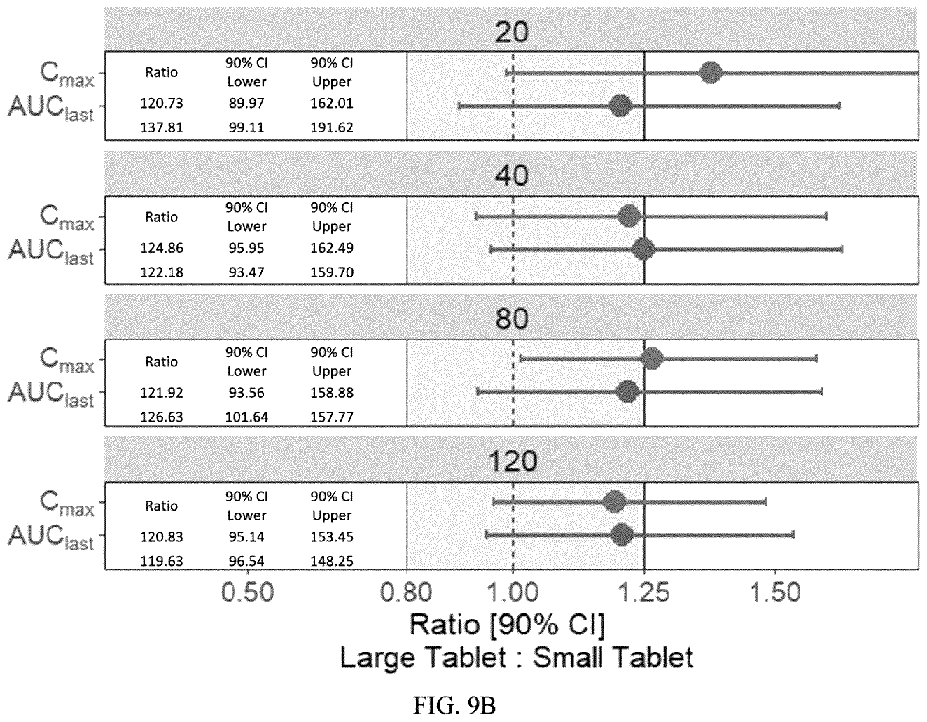

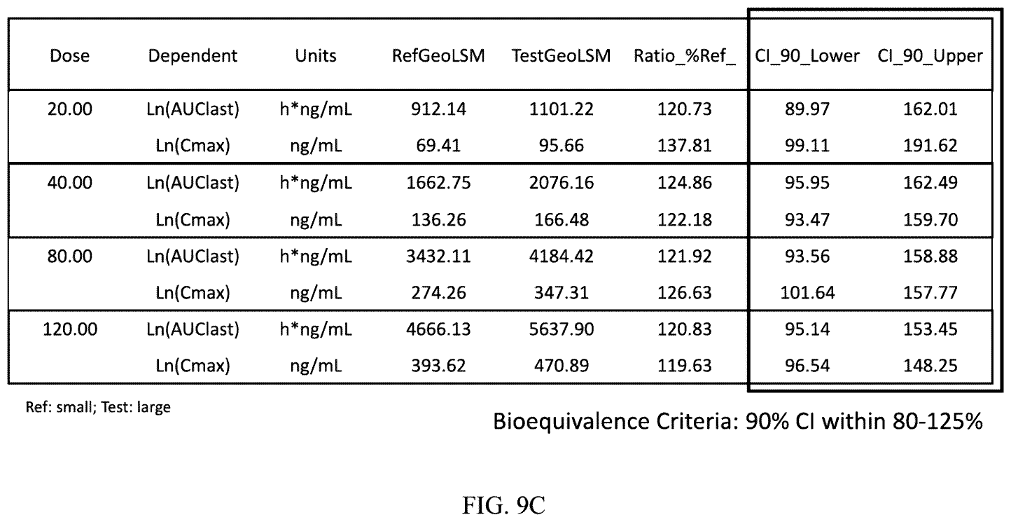

FIGS. 9A-9C depict relative bioavailability by parallel analysis with small, round tablets, or large tablets. While the large tablets appear to have increased exposure relative to the small, round tablets this is likely an artifact of the parallel study design. When the relative bioavailability of 120 mg tablets is assessed by crossover analysis, bioequivalence (BE) criteria is met, while when assessed by parallel analysis, the large tablet has higher exposure than the small, round tablets, by a similar magnitude as the tablets at lower strengths. Outlier subjects excluded from analysis.

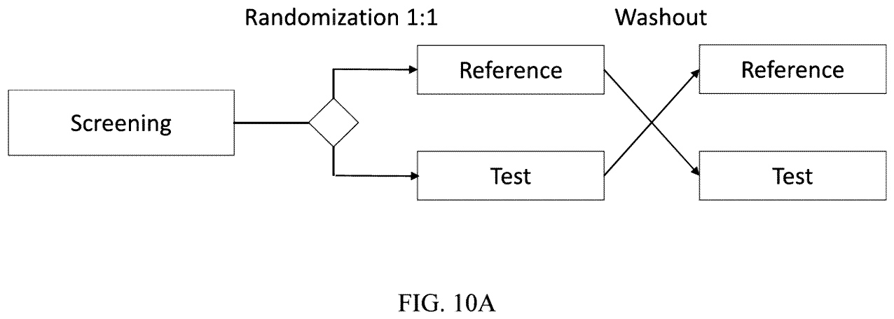

FIG. 10A depicts an exemplary a schematic of a crossover bioequivalence study.

TABLE 5
Schedule of Activities

| Trial Period | | | | | | | | | | | | |
|---|---|---|---|---|---|---|---|---|---|---|---|---|
| Screening/ | | | | | | | | | | | | |
| Baseline | | Intervention | | | | | | | | | | |
| Visit Day (Visit Window in Days) | | | | | | | | | | | | |
| −28 to −2 | −1 | 1 | 2 | 3 | 4 | 5 | 6 | 7 | 8 | 9 | 10 | 11 |

TRIAL ENTRY AND GENERAL ASSESSMENTS

| Informed consent | X | | |
|---|---|---|---|
| Inclusion/exclusion | X | X | |
| criteria | | | |
| Medical history | X | | |
| Demographic data | X | | |
| Body weight/ | X | | |
| height/BMI | | | |
| Urine drug/alcohol | X | X | |
| (ethanol)/nicotine | | | |
| (cotinine) screen | | | |
| Viral serology screen ^{b} | X | | |
| Randomization ^{c} | | | X |
| Inpatient stay | | A | X |
| Telephone call | | | |

SAFETY ASSESSMENTS

| Physical examination ^{d} | X | X | | | | X | | | X | | | X | |
|---|---|---|---|---|---|---|---|---|---|---|---|---|---|
| Clinical laboratory | X | X | | | | X | | | X | | | X | |
| evaluations ^{e} | | | | | | | | | | | | | |
| Pregnancy test ^{f} | X | X | | | | | | | | | | | |
| | (urine) | (serum) | | | | | | | | | | | |
| Vital signs ^{g} | X | X | X | X | X | X | X | X | X | X | X | X | X |
| 12-lead ECG ^{h} | X | X | X | | | X | | | X | | | X | |
| C-SSRS | X | | | | | | | | | | | | |
| (Baseline/Screening) | | | | | | | | | | | | | |
| C-SSRS | | X | | | | | | | | | | | X |
| (since last visit) ⁱ | | | | | | | | | | | | | |
| AE monitoring | X | | | | | | | | | | | | |
| and recording ^{j} | | | | | | | | | | | | | |
| Concomitant meds/ | X | | | | | | | | | | | | |
| procedures ^{j} | | | | | | | | | | | | | |

PHARMACOKINETICS

| | | | |
|---|---|---|---|
| Blood collection | X | X | X |
| for study drug | | | |
| concentration and | | | |
| metabolites ^{k} | | | |

STUDY DRUG

| | | | | | | | | | | | |
|---|---|---|---|---|---|---|---|---|---|---|---|
| Fasted study drug | X | X | X | X | X | X | X | X | X | X | X |
| administration ^{l} | | | | | | | | | | | |
| Fed study drug | | | | | | | | | | | |
| administration ⁿ | | | | | | | | | | | |

| Trial Period | | | | | | | | | |
|---|---|---|---|---|---|---|---|---|---|
| | | | | | | | | | Safety |
| Intervention | | | | | | | | | Follow-up |
| Visit Day (Visit Window in Days) | | | | | | | | | |
| 12 | 13 | 14 | 15 | 16 | 17 | 18/EOT ^{a} | 19 | 20 | 25 (+1)/EOS |

TRIAL ENTRY AND GENERAL ASSESSMENTS

| Informed consent | | | |
|---|---|---|---|
| Inclusion/exclusion | | | |
| criteria | | | |
| Medical history | | | |
| Demographic data | | | |
| Body weight/ | | | |
| height/BMI | | | |
| Urine drug/alcohol | | | |
| (ethanol)/nicotine | | | |
| (cotinine) screen | | | |
| Viral serology screen ^{b} | | | |
| Randomization ^{c} | | | |
| Inpatient stay | X | D | |
| Telephone call | | | X |

SAFETY ASSESSMENTS

| | | | | | | | | | |
|---|---|---|---|---|---|---|---|---|---|
| Physical examination ^{d} | | X | | | X | | X | | X |
| Clinical laboratory | | X | | | X | | X | | |
| evaluations ^{e} | | | | | | | | | |
| Pregnancy test ^{f} | | | | | | | X | | |
| | | | | | | | (urine) | | |
| Vital signs ^{g} | X | X | X | X | X | X | X | X | X |
| 12-lead ECG ^{h} | | X | | | X | | X | | |
| C-SSRS | | | | | | | | | |
| (Baseline/Screening) | | | | | | | | | |
| C-SSRS | X | | | | | X | | X | |
| (since last visit) ⁱ | | | | | | | | | |

| | |
|---|---|
| AE monitoring | X |

and recording ^{j}

| | |
|---|---|
| Concomitant meds/ | X |

procedures ^{j}

PHARMACOKINETICS

| | | | |
|---|---|---|---|
| Blood collection | X | X | X |
| for study drug | | | |
| concentration and | | | |
| metabolites ^{k} | | | |

STUDY DRUG

| | | | | | | | |
|---|---|---|---|---|---|---|---|
| Fasted study drug | X | X ^{m} | X ^{m} | X ^{m} | | | |
| administration ^{l} | | | | | | | |
| Fed study drug | | | | | X | X | X |
| administration ⁿ | | | | | | | |

A = Admission; AE = adverse event; BMI = body mass index; C-SSRS = Columbia-Suicide Severity Rating Scale; D = Discharge; CG = electrocardiogram; EOS = End of Study; EOT = End of Treatment; FSH = follicle-stimulating hormone; hCG = human chorionic gonadotropin; HIV = human immunodeficiency virus.
^{a} If a participant discontinues prior to EOT, an EOT Visit should be completed the same day, and an EOS Visit (follow-up telephone call) should be completed approximately 7 days after the last dose of study drug. If a participant discontinues after EOT, a Discharge (Day 20) Visit should be completed the same day and an EOS Visit (follow-up telephone call) should be completed approximately 7 days after the last dose of study drug.
^{b} Serological assay for HIV, hepatitis B, and hepatitis C.
^{c} Participants randomized in a 1:1 ratio to receive dose titration with either small 20 mg PRAX-944 tablets (Titration Group 1) or large, single-unit dosage PRAX 944 tablets (Titration Group 2).
^{d} Complete physical examination at screening visit and at EOT and safety follow-up visit Day 20; all other physical examinations will be symptom driven.
^{e} Clinical laboratories include complete blood count, clinical chemistry, coagulation (Screening only), urinalysis, and urine albumin to be collected predose on dosing days.
^{f} If applicable, urine hCG at Screening (Days −28 to −2), serum hCG at Day −1, and urine hCG at Day 18/EOT or discharge. If applicable, FSH to be collected at Screening for female participants with amenorrhea ≥1 year but <2 years.
^{g} Oral, temporal, or tympanic temperature; respiratory rate; supine blood pressure; and pulse rate will be obtained on Day 1 predose and 2 hours postdose. After Day 1, the measures will be obtained every 24 hours from the Day 1 predose measurement until the participant is discharged. On days when dosing occurs, the measurement should be taken predose.
^{h} To be recorded after the participant has been supine for at least 5 minutes. Triplicate measurements at Screening only; Baseline: anytime; during the Intervention Period: 2 hours after dosing (±15 mins) on Days 1, 4, 7, 10, 13, 16, and 18.
ⁱ During the Intervention Period, C-SSRS to be conducted predose.
^{j} Throughout the trial from time of signing informed consent until end of trial.
^{k} Collected predose and 0.25, 0.5, 1, 1.5, 2, 2.5, 3, 4, 5, 6, 8, 12, 16, and 24 hours postdose. The 16- and 24-hour timepoints may be collected on the following day, with the 24-hour timepoint collected before dosing on the following day. Sample collection windows are as follows: up to 30 minutes predose, ±2 mins for <1-hour postdose, ±5 mins for 1- to 3-hours postdose, and ±10 mins for 4- to 12-hours postdose.
^{l} Each dose of study drug will be taken with 240 mL of water after an overnight fast. No food or drink, except water, will be allowed for at least 10 hours prior to dosing. Participants are to remain fasted for 4 hours postdose. Participants are not allowed to drink water for 2 hours before and for 1 hour after each dose. Participants will receive the same dosage form (small 20-mg PRAX-944 tablets in Titration Group 1 and large, single-unit 120-mg PRAX-944 tablets in Titration Group 2) on Days 1 through 12.
^{m} On Days 13-15, Titration Group 1 will cross over to receive large, single-unit 120-mg PRAX-944 tablets, and Titration Group 2 will cross over to receive small 20-mg PRAX-944 tablets.
ⁿ All participants will receive 120-mg single-unit dosage PRAX-944 tablet co-administered with a high-fat, high-calorie meal.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permit the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A single-unit dosage form, comprising:
the compound of Formula (I) or a pharmaceutically acceptable salt thereof (e.g., the compound of Formula (II), e.g., PRAX-944 HCl),
wherein the single-unit dosage form is bioequivalent to a reference composition of the same dosage strength administered as one or more dosage forms.

2. The single-unit dosage form of claim 1, wherein the single-unit dosage form and the reference composition have:
(i) a different size;
(ii) a different shape;
(iii) both a different size and a different shape;
(iv) the same shape but different sizes; or
(v) the same size but different shapes.

3. The single-unit dosage form of claim 1, wherein the single-unit dosage form is larger than the reference composition.

4. The single-unit dosage form of claim 3, wherein the reference composition comprises smaller, round 20-mg PRAX-944 tablets.

5. The single-unit dosage form of any one of the preceding claims, wherein:
(i) the single-unit dosage form is a larger dosage form that exhibits bioequivalence upon administration to a subject in a fed state and/or fasted state as compared to administration of a reference composition of the same dosage strength administered as one or more smaller, round 20-mg PRAX-944 tablets to a subject in a fed and/or fasted state;
wherein bioequivalency is established by:
(a) a 90% Confidence Interval for AUC which is between about 80% and about 125%, and
(b) a 90% Confidence Interval for Cmax, which is between about 80% and about 125%.

6. The single-unit dosage form of any one of the preceding claims, which:
(i) reduces the total number of dosage units (e.g., tablets) needed to deliver a specific dosage strength to a subject;
(ii) is characterized by a physical size and shape, (e.g., tablet size and shape) which makes it easier and more convenient to grasp for subjects with a movement disorder, optionally essential tremor (ET);
(iii) can be formulated as single-unit dose strengths for 5 mg, 10 mg, 20 mg, 40 mg, 60 mg, 80 mg, 100 mg, and 120 mg; and/or
(iv) does not require titration to reach doses >40 mg.

7. The single-unit dosage form of any one of the preceding claims, wherein at least about 50% of the compound of Formula (I) or a pharmaceutically acceptable salt thereof (e.g., the compound of Formula (II), e.g., PRAX-944 HCl) is released within about 1 hour to about 12 hours upon administration to a subject, optionally
wherein at least about about 50% of the compound of Formula (I) or a pharmaceutically acceptable salt thereof (e.g., the compound of Formula (II), e.g., PRAX-944 HCl) is released in about 1 hour to about 12 hours using USP apparatus type-I, media containing 900 mL 0.1 M HCl, and a paddle speed of 100 rpm.

8. The single-unit dosage form of any one of the preceding claims, comprising from about 1 mg to about 200 mg of the compound of Formula (I) or a pharmaceutically acceptable salt thereof (e.g., the compound of Formula (II), e.g., PRAX-944 HCl).

9. The single-unit dosage form of any one of the preceding claims, comprising from about 1% by weight to about 70% by weight of the compound of Formula (I) or a pharmaceutically acceptable salt thereof (e.g., the compound of Formula (II), e.g., PRAX-944 HCl).

10. The single-unit dosage form of any one of the preceding claims, further comprising a modified-release polymer.

11. The single-unit dosage form of any one of the preceding claims, wherein the modified-release polymer comprises a matrix polymer, optionally selected from the group consisting of a hydrophilic matrix polymer, a hydrophobic matrix polymer, a polyacrylate polymer, and combinations thereof.

12. The single-unit dosage form of any one of the preceding claims, wherein the modified-release polymer comprises
(i) a hydrophilic matrix polymer, optionally selected from the group consisting of hypromellose, HPMC (hydroxyl-propyl methylcellulose), and combinations thereof, optionally wherein the HPMC (hydroxyl-propyl methylcellulose) is selected from the group consisting of Methocel K4M, Methocel K100LV, Methocel E50LV, and combinations thereof;
(ii) a hydrophobic matrix polymer, optionally selected from the group consisting of ethyl cellulose, ethocel, and combinations thereof; and/or
(iii) a polyacrylate polymer, optionally selected from the group consisting of Eudragit RL100, Eudragit RS100, and combinations thereof.

13. The single-unit dosage form of any one of the preceding claims, comprising from about 5 mg to 300 mg of a modified-release polymer.

14. The single-unit dosage form of any one of the preceding claims, comprising from about 10% by weight to about 70% by weight of the modified-release polymer.

15. The single-unit dosage form of any one of the preceding claims, wherein the modified-release polymer is hypromellose.

16. The single-unit dosage form of any one of the preceding claims, further comprising a diluent.

17. The single-unit dosage form of any one of the preceding claims, wherein the diluent comprises:
(i) a cellulose derivative, optionally a microcrystalline cellulose, optionally a silicified microcrystalline cellulose;
(ii) a starch, optionally selected from the group consisting of a hydrolyzed starch, a pregelatinized starch, and combinations thereof);
(iii) an anhydrous lactose;
(iv) a lactose monohydrate;
(v) a di-calcium phosphate (DCP); and/or
(vi) a sugar alcohol, optionally selected from the group consisting of sorbitol, xylitol, mannitol, and combinations thereof.

18. The single-unit dosage form of any one of the preceding claims, comprising from about 5 mg to about 300 mg of diluent.

19. The single-unit dosage form of any one of the preceding claims, comprising from about 5% by weight to about 50% by weight of diluent.

20. The single-unit dosage form of any one of the preceding claims, wherein the diluent is microcrystalline cellulose, optionally silicified microcrystalline cellulose.

21. The single-unit dosage form of any one of the preceding claims, wherein the diluent is a sugar alcohol, optionally mannitol.

22. The single-unit dosage form of any one of the preceding claims, further comprising a glidant.

23. The single-unit dosage form of any one of the preceding claims, wherein the glidant is selected from the group consisting of fumed silica, optionally colloidal silicon dioxide, talc, magnesium carbonate, and combinations thereof.

24. The single-unit dosage form of any one of the preceding claims, comprising from about 1 mg to about 10 mg of glidant.

25. The single-unit dosage form of any one of the preceding claims, comprising from about 1% by weight to about 10% by weight of glidant.

26. The single-unit dosage form of any one of the preceding claims, wherein the glidant is fumed silica, optionally colloidal silicon dioxide.

27. The single-unit dosage form of any one of the preceding claims, further comprising a lubricant.

28. The single-unit dosage form of any one of the preceding claims, wherein the lubricant is selected from the group consisting of magnesium stearate, calcium stearate, stearic acid, talc, silica, a fat, optionally vegetable stearin, and combinations thereof.

29. The single-unit dosage form of any one of the preceding claims, comprising from about 1 mg to about 10 mg of lubricant.

30. The single-unit dosage form of any one of the preceding claims, comprising from about 1% by weight to about 10% by weight of lubricant.

31. The single-unit dosage form of any one of the preceding claims, wherein the lubricant comprises magnesium stearate.

32. The single-unit dosage form of any one of the preceding claims, further comprising a coating.

33. The single-unit dosage form of any one of the preceding claims, wherein the coating comprises a film coating agent.

34. The single-unit dosage form of any one of the preceding claims, wherein the coating comprises compendial grade polyvinyl alcohol, titanium dioxide, polyethylene glycol 3350, and/or talc.

35. The single-unit dosage form of any one of the preceding claims, comprising from about 1 mg to about 20 mg of coating.

36. The single-unit dosage form of any one of the preceding claims, comprising from about 1% by weight to about 10% by weight of coating.

37. The single-unit dosage form of any one of the preceding claims, wherein the coating comprises Opadry® II white 85F18422.

38. The single-unit dosage form of any one of the preceding claims, which is a tablet, optionally formulated for oral administration.

39. The single-unit dosage form of any one of the preceding claims, which is an oblong tablet, an oval tablet, or a capsule-shaped tablet, optionally wherein the tablet is not a round tablet.

40. The single-unit dosage form of any one of the preceding claims, which has a total weight from about 200 mg to about 600 mg per dosage unit (e.g., per tablet).

41. The single-unit dosage form of any one of the preceding claims, which has a length of about 1 mm to about 30 mm.

42. The single-unit dosage form of any one of the preceding claims, which has a width of about 1 mm to about 10 mm.

43. The single-unit dosage form of any one of the preceding claims, which has a length of about 14 to about 16 mm, and a width of about 5 mm to about 7 mm.

44. The single-unit dosage form of any one of the preceding claims, comprising PRAX-944 HCl in an amount equivalent to about 5.0 mg of PRAX-944 free base per dosage unit (e.g., per tablet).

45. The single-unit dosage form of any one of the preceding claims, comprising PRAX-944 HCl in an amount equivalent to about 10.0 mg of PRAX-944 free base per dosage unit (e.g., per tablet).

46. The single-unit dosage form of any one of the preceding claims, comprising PRAX-944 HCl in an amount equivalent to about 20.0 mg of PRAX-944 free base per dosage unit (e.g., per tablet).

47. The single-unit dosage form of any one of the preceding claims, comprising from about 1% by weight to about 10% by weight of PRAX-944 HCl, optionally from about 15 mg to about 25 mg of PRAX-944 HCl per dosage unit (e.g., per tablet).

48. The single-unit dosage form of any one of the preceding claims, comprising PRAX-944 HCl in an amount equivalent to about 40.0 mg of PRAX-944 free base per dosage unit (e.g., per tablet).

49. The single-unit dosage form of any one of the preceding claims, comprising from about 5% by weight to about 15% by weight of PRAX-944 HCl, optionally from about 40 mg to about 50 mg of PRAX-944 HCl per dosage unit (e.g., per tablet).

50. The single-unit dosage form of any one of the preceding claims, comprising PRAX-944 HCl in an amount equivalent to about 80.0 mg of PRAX-944 free base per dosage unit (e.g., per tablet).

51. The single-unit dosage form of any one of the preceding claims, comprising from about 15% by weight to about 25% by weight of PRAX-944 HCl, optionally from about 85 mg to about 95 mg of PRAX-944 HCl per dosage unit (e.g., per tablet).

52. The single-unit dosage form of any one of the preceding claims, comprising PRAX-944 HCl in an amount equivalent to about 120.0 mg of PRAX-944 free base per dosage unit (e.g., per tablet).

53. The single-unit dosage form of any one of the preceding claims, comprising from about 25% by weight to about 35% by weight of PRAX-944 HCl, optionally from about 125 mg to about 135 mg of PRAX-944 HCl per dosage unit (e.g., per tablet).

54. The single-unit dosage form of any one of the preceding claims, further comprising one or more of
(i) a modified-release polymer, optionally from about 35% by weight to about 45% by weight of modified-release polymer, optionally from about 175 mg to about 185 mg of modified-release polymer per dosage unit (e.g., per tablet);
(i) a soluble diluent, optionally from about 6% by weight to about 10% by weight of soluble diluent, optionally from about 34 mg to about 38 mg of soluble diluent per dosage unit (e.g., per tablet);
(iii) a glidant, optionally from about 1% by weight to about 3% by weight of glidant, optionally from about 6 mg to about 8 mg of glidant per dosage unit (e.g., per tablet);
(iv) a lubricant, optionally from about 0.5% by weight to about 2% by weight of lubricant, optionally from about 3 mg to about 6 mg of lubricant per dosage unit (e.g., per tablet); and
(v) a coating, optionally from about 12% by weight to about 15% by weight of coating, optionally from about 1 mg to about 5 mg of coating per dosage unit (e.g., per tablet).

55. The single-unit dosage form of any one of the preceding claims, comprising a formulation as set forth in any one of Tables 6-16, optionally comprising:
(i) large tablet prototype 01 (58% K4M Round) (DC);
(ii) large tablet prototype 01 (58% K4M Caplet) (DC);
(iii) large tablet prototype 04 (58% K4M) (DG);
(iv) large tablet prototype 23 (50% K4M);
(v) large tablet prototype 28 (58% K100LV);
(vi) large tablet prototype 34 (50% K100LV, 8% Mannitol EG);
(vii) large tablet prototype 36 (50% K100LV, 8% Mannitol IG);
(viii) large tablet prototype 52 (28% K100LV, 30% E50LV);
(ix) large tablet prototype 53 (40% K100LV, 8% Mannitol IG); or
(x) large tablet prototype 54 (46% K100LV, 12% Mannitol IG).

56. The single-unit dosage form of any one of the preceding claims, comprising large tablet prototype 53 (40% K100LV, 8% Mannitol IG).

57. A pharmaceutical composition comprising the single-unit dosage form of any one of the preceding claims, and at least one pharmaceutically acceptable carrier or excipient.

58. A method of treating a disease or condition relating to aberrant function or activity of a T-type calcium channel in a subject in need thereof, comprising administering to the subject a therapeutically effective amount of the single-unit dosage form of any one of claims 1-56, or the pharmaceutical composition of claim 57.

59. A method of treating a tremor in a subject in need thereof, comprising administering to the subject a therapeutically effective amount of the single-unit dosage form of any one of claims 1-56, or the pharmaceutical composition of claim 57.

60. A method of treating an essential tremor in a subject in need thereof, comprising administering to the subject a therapeutically effective amount of the single-unit dosage form of any one of claims 1-56, or the pharmaceutical composition of claim 57.

61. The method of claim 60, which results in reduction of the essential tremor as assessed by The Essential Tremor Rating Assessment Scale (TETRAS) score.

62. The method of claim 60 or 61, wherein the reduction of the essential tremor is assessed by The Essential Tremor Rating Assessment Scale (TETRAS) upper limb score.

63. The method of any one of claims 60-62, wherein the reduction of the essential tremor is assessed by TETRAS-ADL (activities of daily living).

64. The method of any one of claims 60-63, wherein the reduction of the essential tremor is assessed by TETRAS performance subscale score or TETRAS performance individual items.

65. The method of any one of claims 60-64, which results in reduction of the essential tremor as assessed by accelerometer-based upper limb score.

66. The method of any one of claims 60-65, which results in reduction of sigma frequency band.

67. The method of any one of claims 60-66, wherein the essential tremor is upper limb tremor.
